SEQUENCE LISTING (49 sequences)

SEQ ID NO: 1 - protein, 536 aa, Artificial sequence
  Amino-acid sequence of PPO1 from wild-type rice (OsPPO1 WT)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 2 - protein, 537 aa, Artificial sequence
  Amino-acid sequence of PPO1 from wild-type corn (ZmPPO1 WT)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLLNYIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 3 - protein, 537 aa, Artificial sequence
  Amino-acid sequence of PPO1 wild-type from oilseed rape (BnPPO1-C5)
MDLSLLRPQPFLSPFSNPFPRSRPYKPPNLRCSVSGGSVVGGSSTIEGGRGGKTVAADCVIVGGGISGLCIAQALVTKHPDAAKSVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPDAISARLGDKVKVSWKLSSISKLPSGGYSLTYETPEGIVTVQSKSVVMTVPSHVASSLLRPLSDSAAEALSKLYYPPVAAVSISYPKEAIRSECLIDGELKGFGQLHPRTQKVETLGTIYSSSLFPNRAPPGRVLLLNYIGGATNTGILSKSEGELVEAVDRDLRKMLIKPSSTDPLVLGVKVWPQAIPQFLIGHIDLVDAAKASLSSSGHEGLFLGGNYVAGVALGRCVEGAYETATQVNDFMSRYAYK

SEQ ID NO: 4 - protein, 536 aa, Artificial sequence
  Amino-acid sequence of PPO1 from wild-type oilseed rape (BnPPO1-A10)
MDLSLLRPQPFLSPFSNPFPRSRPYKPLNLRCSVSGGSVVGSSTIEGGGGGKTVTADCVIVGGGISGLCIAQALVTKHPDAAKNVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQAKNKAPKTTRDPRLPKPKGQTVGSFRKGLTMLPEAISARLGDKVKVSWKLSSITKLASGEYSLTYETPEGIVTVQSKSVVMTVPSHVASSLLRPLSDSAAEALSKLYYPPVAAVSISYAKEAIRSECLIDGELKGFGQLHPRTQKVETLGTIYSSSLFPNRAPPGRVLLLNYIGGATNTGILSKSEGELVEAVDRDLRKMLIKPSSTDPLVLGVKLWPQAIPQFLIGHIDLVDAAKASLSSSGHEGLFLGGNYVAGVALGRCVEGAYETATQVNDFMSRYAYK

SEQ ID NO: 5 - protein, 558 aa, Artificial sequence
  Amino-acid sequence of PPO1 from wild-type peanut (AhPPO1-A)
MPAMLTLPNDTVFLQNTMHSPTSSFSPHSPLIIPFPSPCRHPSTTKFPLIRCSLAGESSTASPAKPAALPRSAVGASVDCVVVGGGISGLCIAQALSTKHAKAAANVVVTEARDRVGGNITTIERDGYLWEEGPNSFQPSDPMLTMVVDSGLKDELVLGDPNAPRFVLWNGELRPVPGKPTDLPFFDLMSFGGKLRAGFGALGIRPPPPGHEESVEEFVRRNLGDEVFERLIEPFCSGVYAGDPSKLSMKAAFGKVWRLEQNGGSIIGGTFKAIQERKSTSKPPRDPRLPTPKGQTVGSFRKGLTMLPEAISAKLGNKVKLSWKLLGISKLDSGEYSLTYDTPEGVVSLQTKSVVMTIPSHVASTLLRSISDAAADALSKFYYPPVAAVSVSYPKEAIRSECLIDGELKGFGQLHPRTQGVETLGTIYSSSLFPNRAPDGRVLLLNYIGGATNPGILSKTDSELVEAVDRDLRKMLINPNAGDPFALGVRVWPQAIPQFLVGHLDLLDIAKSSLKNTGFEGLFLGGNYVSGVALGRCVEGAYEIAAEVDDFLAQKVYK

SEQ ID NO: 6 - protein, 552 aa, Artificial sequence
  Amino-acid sequence of PPO1 from wild-type peanut (AhPPO1-B)
MSAMLTLPNDTVFLQNTMHSPTSSFSPHSPLIIPFPSPCRHPSTTKFPLIRCSLAGESSTASLPRSAVGASVDCVVVGGGISGLCIAQALSTKHAKAAANVVVTEARDRVGGNITTVERDGYLWEEGPNSFQPSDPMLTMVVDSGLKDELVLGDPNAPRFVLWNGRLRPVPGKPTDLPFFDLMSFGGKLRAGFGALGIRPPPPGHEESVEEFVRRNLGDEVFERLIEPFCSGVYAGDPSKLSMKAAFGKVWRLEQNGGSIIGGTFKAIQERKSTSKPPRDPRLPTPKGQTVGSFRKGLIMLPEAISAKLGNKVKLSWKLLGISKLDSGEYSLTYDTPEGVVSLQTKSVVMTIPSHVASTLLRSISETAADALSKFYYPPVAAVSVSYPKEAIRSECLIDGELKGFGQLHPRTQGVETLGTIYSSSLFPNRAPDGRVLLLNYIGGATNPGILSKTDSELVEAVDRDLRKMLINPNAGDPFALGVRVWPQAIPQFLVGHLDLLDIAKSSLKNTGFEGLFLGGNYVSGVALGRCVEGAYEIAAEVDDFLAQKVYK

SEQ ID NO: 7 - protein, 543 aa, Artificial sequence
  Amino-acid sequence of PPO1 from wild-type soybean (GmPPO1)
MVSVFNEILFPPNQTLLRPSLHSPTSFFTSPTRKFPRSRPNPILRCSIAEESTASPPKTRDSAPVDCVVVGGGVSGLCIAQALATKHANANVVVTEARDRVGGNITTMERDGYLWEEGPNSFQPSDPMLTMVVDSGLKDELVLGDPDAPRFVLWNRKLRPVPGKLTDLPFFDLMSIGGKIRAGFGALGIRPPPPGHEESVEEFVRRNLGDEVFERLIEPFCSGVYAGDPSKLSMKAAFGKVWKLEKNGGSIIGGTFKAIQERNGASKPPRDPRLPKPKGQTVGSFRKGLTMLPDAISARLGNKVKLSWKLSSISKLDSGEYSLTYETPEGVVSLQCKTVVLTIPSYVASTLLRPLSAAAADALSKFYYPPVAAVSISYPKEAIRSECLIDGELKGFGQLHPRSQGVETLGTIYSSSLFPNRAPPGRVLLLNYIGGATNTGILSKTDSELVETVDRDLRKILINPNAQDPFVVGVRLWPQAIPQFLVGHLDLLDVAKASIRNTGFEGLFLGGNYVSGVALGRCVEGAYEVAAEVNDFLTNRVYK

SEQ ID NO: 8 - protein, 536 aa, Artificial sequence
  Amino-acid sequence of PPO1 from wild-type sorghum(SbPPO1)
MVAAAAMATAASAAAPLLNGTRRPARLRRRGLRVRCAAVAGGAAEAPASTGARLSADCVVVGGGISGLCTAQALATRHGVGEVLVTEARARPGGNITTVERPEEGYLWEEGPNSFQPSDPVLSMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEEAGGSIIGGTIKTIQERGKNPKPPRDPRLPKPKGQTVASFRKGLAMLPNAITSSLGSKVKLSWKLTSITKSDGKGYVLEYETPEGVVLVQAKSVIMTIPSYVASDILRPLSGDAADALSRFYYPPVAAVTVSYPKEAIRKECLIDGELQGFGQLHPRSQGVETLGTIYSSSLFPNRAPAGRVLLLNYIGGATNTGIVSKTESELVEAVDRDLRKMLINSTAVDPLVLGVRVWPQAIPQFLVGHLDLLEVAKSALDQGGYDGLFLGGNYVAGVALGRCIEGAYESAAQIYDFLTKYAYK

SEQ ID NO: 9 - protein, 531 aa, Artificial sequence
  Amino-acid sequence of PPO1 from wild-type wheat (TaPPO1-A)
MAGATMATATVTAASPLRGRVTGRPRRVRTRCAAASGATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVEAVDRDLRKMLINPRAADPLALGVRVWPQAIPQFLIGHLDRLAAAKSALGRGGYDGLFLGGNYVAGVALGRCIEGAYESASQVSDFLTKYAYK

SEQ ID NO: 10 - protein, 531 aa, Artificial sequence
  Amino-acid sequence of PPO1 from wild-type wheat (TaPPO1-B)
MAGATMATATVAAASPLRGRVTGRPHRVRPRCATASSATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVGAVDRDLRKMLINPRAADPLALGVRVWPQAIPQFLIGHLDRLAAAKSALGQGGYDGLFLGGNYVAGVALGRCIEGAYESASQVSDFLTKYAYK

SEQ ID NO: 11 - protein, 531 aa, Artificial sequence
  Amino-acid sequence of PPO1 from wild-type wheat (TaPPO1-D)
MAGATMATATVTAASPLRGRVTGRPRRVRPRCAAASGATETPAAPGVRLSADCVIVGAGISGLCTAQALATRYGVGDLLITEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVEAVDSDLRKMLINPRAADPLALGVRVWPQAIPQFLIGHLDRLAAAKSALGRGGYDGLFLGGNYVAGVALGRCIEGAYESASQVSDFLTKYAYK

SEQ ID NO: 12 - protein, 558 aa, Artificial sequence
  Amino-acid sequence of PPO1 form wild-type tomato (SlPPO1)
MTTTAVVNHPSIFTHRSPLPSPSSSSSSSPSFLFLNRTNFIPYFSTSKRSSVNCNGWRTRCSVAKNYTVPPSEVDGNQLPELDCVVVGAGISGLCIAKVISANYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIESFCSGVYAGDPSKLSMKAAFGKVWKLEQTGGSIIGGTFKAIKERSSNPKPPRDPRLPTPKGQTVGSFRKGLRMLPDAICERLGSKVKLSWKLSSITKSDKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLLNYIGGATNTEIVSKTESQLVEAVDRDLRKMLIKPKAQDPFVTGVRVWPQAIPQFLVGHLDTLGTAKAALSDNGLDGLFLGGNYVSGVALGRCVEGAYEIASEVTGFLSQYAYK

SEQ ID NO: 13 - protein, 557 aa, Artificial sequence
  Amino-acid sequence of PPO1 from wild-type potato (StPPO1)
MTTTAVANHPSIFTHRSPLLSPSSSSSSPSFLFLNRTNFIPYFSTSKRNSVNCNGWRTRCSVAKDYTVPPSEVDGNQFPELDCVVVGAGISGLCITKVISDNYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIEPFCSGVYAGDPSKLSMKAAFGKVWKLEQTGGSIIGGTFKAIKERSSNPKPPRDPRLPTPKGQTVGSFRKGLRMLPDAICERLGSKVKLSWKLSSITKSEKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLLNYIGGATNTEIVSKTESQLVEAVDRDLRKMLIKPKAQDPFVTGVRVWPQAIPQFLVGHLDTLGTAKTALSDNGHDGLFLGGNYVSGVALGRCVEGAYEIASEVTGFLSQYAYK

SEQ ID NO: 14 - protein, 553 aa, Artificial sequence
  Amino-acid sequence of PPO1 form wild-type tobacco (NtPPO1)
MTTTPIANHPNVFTHRSPPSSSSSSPSAFLTRTSFLPFSSICKRNSVNCTGWRTRCSVAKDYTVPSSAVDGGPAAELDCVIVGAGISGLCIAQVMSANYPNLMVTEARDRAGGNITTVERDGYLWEEGPNSFQPSDPMLTMAVDCGLKDDLVLGDPNAPRFVLWKGKLRPVPSKLTDLPFFDLMSIPGKLRAGFGAIGLRPSPPGHEESVEQFVRRNLGGEVFERLIEPFCSGVYAGDPSKLSMKAAFGKVWKLEETGGSIIGGTFKAIKERSSTPKAPRDPRLPKPKGQTVGSFRKGLRMLPDAISARLGSKLKLSWKLSSITKSEKGGYRLTYETPEGVVSLQSRSIVMTVPSYVASNILRPLSVAAADALSNFYYPPVGAVTISYPQEAIRDERLVDGELKGFGQLHPRTQGVETLGTIYSSSLFPNRAPKGRVLLLNYIGGAKNPEILSKTESQLVEVVDRDLRKMLIKPKAQDPLVVGVRVWPQAIPQFLVGHLDTLSTAKAAMSDNGLEGLFLGGNYVSGVALGRCVEGAYEVASEVTGFLPRYAYK

SEQ ID NO: 15 - protein, 537 aa, Artificial sequence
  Amino-acid sequence of PPO1 from wild-type Arabidopsis thaliana (AtPPO1)
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 16 - protein, 539 aa, Artificial sequence
  Amino-acid sequence of PPO1 from wild-type upland cotton (GhPPO1)
MTALIDLSLLRSSPSVSPFSIPHHQHPPRFRKPFKLRCSLAEGPTISSSKIDGGESSIADCVIVGGGISGLCIAQALATKHRDVASNVIVTEARDRVGGNITTVERDGYLWEEGPNSFQPSDPILTMAVDSGLKDDLVLGDPNAPRFVLWEGKLRPVPSKPTDLPFFDLMSIAGKLRAGFGAIGIRPPPPGYEESVEEFVRRNLGAEVFERFIEPFCSGVYAGDPSKLSMKAAFGRVWKLEEIGGSIIGGTFKTIQERNKTPKPPRDPRLPKPKGQTVGSFRKGLTMLPEAIANSLGSNVKLSWKLSSITKLGNGGYNLTFETPEGMVSLQSRSVVMTIPSHVASNLLHPLSAAAADALSQFYYPPVASVTVSYPKEAIRKECLIDGELKGFGQLHPRSQGIETLGTIYSSSLFPNRAPSGRVLLLNYIGGATNTGILSKTEGELVEAVDRDLRKMLINPNAKDPLVLGVRVWPKAIPQFLVGHLDLLDSAKMALRDSGFHGLFLGGNYVSGVALGRCVEGAYEVAAEVKEFLSQYAYK

SEQ ID NO: 17 - protein, 538 aa, Artificial sequence
  Amino-acid sequence of PPO1 from wild-type radish (RsPPO1)
MDLSLLRPQPFLSPFSNPFPRSRPYYKPLNLHCSVSGGSVVGSSTIEGGGGGGKTVAADCVIVGGGISGLCIAQALVTKHPDAAKNVIVTEAKDRVGGNIITREEQGFLWEEGPNSFQPSDPMLTMVVDSGLKDDLVLGDPTAPRFVLWNGKLRPVPSKLTDLPFFDLMSIRGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPEAISARLGNKVKVSWKLSSITKLANGEYSLTYETPEGIVTVQSKSVVMTVPSHVASSLLRPLSDSAAEALSKLYYPPVAAVSISYPKEAIRSECLIDGELKGFGQLHPRTQKVETLGTIYSSSLFPNRAPPGRVLLLNYIGGTTNTGILSKSEGELVEAVDRDLRKMLIKPSSTDPLVLGVRVWPQAIPQFLIGHIDLVDAAKASLSSSGHEGIFLGGNYVAGVALGRCVECAYETATQVNDFMSRYTYK

SEQ ID NO: 18 - protein, 535 aa, Artificial sequence
  Amino-acid sequence of PPO1 from wild-type foxtail millet (SiPPO1)
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 19 - protein, 537 aa, Artificial sequence
  Amino-acid sequence of PPO1 form wild-type cabbage (BoPPO1)
MDLSLLRPQPFLSPFSNPFPRSRPYKPPNLRCSVSGGSVVGGSSTIEGGGGGKTVAADCVIVGGGISGLCIAQALVTKHPDAAKSVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPEAISARLGDKVKVSWKLSSISKLPSGGYSLTYETPEGIVTVQSKSVVMTVPSHVASSLLRPLSDSAAEALSKLYYPPVAAVSISYLKEAIRSECLIDGELKGFGQLHPRTQKVETLGTIYSSSLFPNRAPPGRVLLLNYIGGATNTGILSKSEGELVEAVDRDLRKMLIMPSSTDPLVLGVKVWPQAIPQFLIGHIDLVDAAKASLSSSGHEGLFLGGNYVAGVALGRCVEGAYETATQVNDFMSRYAYK

SEQ ID NO: 20 - protein, 536 aa, Artificial sequence
  Amino-acid sequence of PPO1 from rice mutant (OsPPO1 L423S)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLSNYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 21 - protein, 536 aa, Artificial sequence
  Amino-acid sequence of PPO1 from rice mutant (OsPPO1 L423I)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLINYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 22 - protein, 536 aa, Artificial sequence
  Amino-acid sequence of PPO1 from rice mutant (OsPPO1 L423G)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLGNYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 23 - protein, 536 aa, Artificial sequence
  Amino-acid sequence of PPO1 from rice mutant (OsPPO1 Y425M)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNMIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 24 - protein, 536 aa, Artificial sequence
  Amino-acid sequence of PPO1 from rice mutant (OsPPO1 Y425I)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNIIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 25 - protein, 536 aa, Artificial sequence
  Amino-acid sequence of PPO1 from rice mutant (OsPPO1 Y425V)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNVIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 26 - protein, 536 aa, Artificial sequence
  Amino-acid sequence of PPO1 from rice mutant (OsPPO1 L423S/Y425I)
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLSNIIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 27 - protein, 537 aa, Artificial sequence
  Amino-acid sequence of PPO1 from corn mutant (ZmPPO1 L424T/Y426V)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLTNVIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 28 - protein, 537 aa, Artificial sequence
  Amino-acid sequence of PPO1 form corn mutant (ZmPPO1 L424S/Y426V)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLSNVIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 29 - protein, 537 aa, Artificial sequence
  Amino-acid sequence of PPO1 from corn mutant (ZmPPO1 L424V/Y426L)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLVNLIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 30 - protein, 537 aa, Artificial sequence
  Amino-acid sequence of PPO1 from corn mutant (ZmPPO1 L424W/Y426L)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLWNLIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 31 - protein, 537 aa, Artificial sequence
  Amino-acid sequence of PPO1 form corn mutant (ZmPPO1 L424S/Y426I)
MVAATATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLLWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLSNIIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 32 - protein, 537 aa, Artificial sequence
  Amino-acid sequence of PPO1 from oilseed rape mutant (BnPPO1-C5 L424S/Y426I)
MDLSLLRPQPFLSPFSNPFPRSRPYKPPNLRCSVSGGSVVGGSSTIEGGRGGKTVAADCVIVGGGISGLCIAQALVTKHPDAAKSVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPDAISARLGDKVKVSWKLSSISKLPSGGYSLTYETPEGIVTVQSKSVVMTVPSHVASSLLRPLSDSAAEALSKLYYPPVAAVSISYPKEAIRSECLIDGELKGFGQLHPRTQKVETLGTIYSSSLFPNRAPPGRVLLSNIIGGATNTGILSKSEGELVEAVDRDLRKMLIKPSSTDPLVLGVKVWPQAIPQFLIGHIDLVDAAKASLSSSGHEGLFLGGNYVAGVALGRCVEGAYETATQVNDFMSRYAYK

SEQ ID NO: 33 - protein, 536 aa, Artificial sequence
  Amino-acid sequence of PPO1 from oilseed rape mutant (BnPPO1-A10 L423S/Y425I)
MDLSLLRPQPFLSPFSNPFPRSRPYKPLNLRCSVSGGSVVGSSTIEGGGGGKTVTADCVIVGGGISGLCIAQALVTKHPDAAKNVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQAKNKAPKTTRDPRLPKPKGQTVGSFRKGLTMLPEAISARLGDKVKVSWKLSSITKLASGEYSLTYETPEGIVTVQSKSVVMTVPSHVASSLLRPLSDSAAEALSKLYYPPVAAVSISYAKEAIRSECLIDGELKGFGQLHPRTQKVETLGTIYSSSLFPNRAPPGRVLLSNIIGGATNTGILSKSEGELVEAVDRDLRKMLIKPSSTDPLVLGVKLWPQAIPQFLIGHIDLVDAAKASLSSSGHEGLFLGGNYVAGVALGRCVEGAYETATQVNDFMSRYAYK

SEQ ID NO: 34 - protein, 558 aa, Artificial sequence
  Amino-acid sequence of PPO1 from peanut mutant (AhPPO1-A L445S/Y447I)
MPAMLTLPNDTVFLQNTMHSPTSSFSPHSPLIIPFPSPCRHPSTTKFPLIRCSLAGESSTASPAKPAALPRSAVGASVDCVVVGGGISGLCIAQALSTKHAKAAANVVVTEARDRVGGNITTIERDGYLWEEGPNSFQPSDPMLTMVVDSGLKDELVLGDPNAPRFVLWNGELRPVPGKPTDLPFFDLMSFGGKLRAGFGALGIRPPPPGHEESVEEFVRRNLGDEVFERLIEPFCSGVYAGDPSKLSMKAAFGKVWRLEQNGGSIIGGTFKAIQERKSTSKPPRDPRLPTPKGQTVGSFRKGLTMLPEAISAKLGNKVKLSWKLLGISKLDSGEYSLTYDTPEGVVSLQTKSVVMTIPSHVASTLLRSISDAAADALSKFYYPPVAAVSVSYPKEAIRSECLIDGELKGFGQLHPRTQGVETLGTIYSSSLFPNRAPDGRVLLSNIIGGATNPGILSKTDSELVEAVDRDLRKMLINPNAGDPFALGVRVWPQAIPQFLVGHLDLLDIAKSSLKNTGFEGLFLGGNYVSGVALGRCVEGAYEIAAEVDDFLAQKVYK

SEQ ID NO: 35 - protein, 552 aa, Artificial sequence
  Amino-acid sequence of PPO1 from peanut mutant (AhPPO1-B L439S/Y441I)
MSAMLTLPNDTVFLQNTMHSPTSSFSPHSPLIIPFPSPCRHPSTTKFPLIRCSLAGESSTASLPRSAVGASVDCVVVGGGISGLCIAQALSTKHAKAAANVVVTEARDRVGGNITTVERDGYLWEEGPNSFQPSDPMLTMVVDSGLKDELVLGDPNAPRFVLWNGRLRPVPGKPTDLPFFDLMSFGGKLRAGFGALGIRPPPPGHEESVEEFVRRNLGDEVFERLIEPFCSGVYAGDPSKLSMKAAFGKVWRLEQNGGSIIGGTFKAIQERKSTSKPPRDPRLPTPKGQTVGSFRKGLIMLPEAISAKLGNKVKLSWKLLGISKLDSGEYSLTYDTPEGVVSLQTKSVVMTIPSHVASTLLRSISETAADALSKFYYPPVAAVSVSYPKEAIRSECLIDGELKGFGQLHPRTQGVETLGTIYSSSLFPNRAPDGRVLLSNIIGGATNPGILSKTDSELVEAVDRDLRKMLINPNAGDPFALGVRVWPQAIPQFLVGHLDLLDIAKSSLKNTGFEGLFLGGNYVSGVALGRCVEGAYEIAAEVDDFLAQKVYK

SEQ ID NO: 36 - protein, 543 aa, Artificial sequence
  Amino-acid sequence of PPO1 from soybean mutant (GmPPO1 L430S/Y432I)
MVSVFNEILFPPNQTLLRPSLHSPTSFFTSPTRKFPRSRPNPILRCSIAEESTASPPKTRDSAPVDCVVVGGGVSGLCIAQALATKHANANVVVTEARDRVGGNITTMERDGYLWEEGPNSFQPSDPMLTMVVDSGLKDELVLGDPDAPRFVLWNRKLRPVPGKLTDLPFFDLMSIGGKIRAGFGALGIRPPPPGHEESVEEFVRRNLGDEVFERLIEPFCSGVYAGDPSKLSMKAAFGKVWKLEKNGGSIIGGTFKAIQERNGASKPPRDPRLPKPKGQTVGSFRKGLTMLPDAISARLGNKVKLSWKLSSISKLDSGEYSLTYETPEGVVSLQCKTVVLTIPSYVASTLLRPLSAAAADALSKFYYPPVAAVSISYPKEAIRSECLIDGELKGFGQLHPRSQGVETLGTIYSSSLFPNRAPPGRVLLSNIIGGATNTGILSKTDSELVETVDRDLRKILINPNAQDPFVVGVRLWPQAIPQFLVGHLDLLDVAKASIRNTGFEGLFLGGNYVSGVALGRCVEGAYEVAAEVNDFLTNRVYK

SEQ ID NO: 37 - protein, 536 aa, Artificial sequence
  Amino-acid sequence of PPO1 from sorghum mutant (SbPPO1 L423S/Y425I)
MVAAAAMATAASAAAPLLNGTRRPARLRRRGLRVRCAAVAGGAAEAPASTGARLSADCVVVGGGISGLCTAQALATRHGVGEVLVTEARARPGGNITTVERPEEGYLWEEGPNSFQPSDPVLSMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEEAGGSIIGGTIKTIQERGKNPKPPRDPRLPKPKGQTVASFRKGLAMLPNAITSSLGSKVKLSWKLTSITKSDGKGYVLEYETPEGVVLVQAKSVIMTIPSYVASDILRPLSGDAADALSRFYYPPVAAVTVSYPKEAIRKECLIDGELQGFGQLHPRSQGVETLGTIYSSSLFPNRAPAGRVLLSNIIGGATNTGIVSKTESELVEAVDRDLRKMLINSTAVDPLVLGVRVWPQAIPQFLVGHLDLLEVAKSALDQGGYDGLFLGGNYVAGVALGRCIEGAYESAAQIYDFLTKYAYK

SEQ ID NO: 38 - protein, 531 aa, Artificial sequence
  Amino-acid sequence of PPO1 form wheat mutant (TaPPO1-A L418S/Y420I)
MAGATMATATVTAASPLRGRVTGRPRRVRTRCAAASGATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLSNIIGGSTNTGIVSKTESDLVEAVDRDLRKMLINPRAADPLALGVRVWPQAIPQFLIGHLDRLAAAKSALGRGGYDGLFLGGNYVAGVALGRCIEGAYESASQVSDFLTKYAYK

SEQ ID NO: 39 - protein, 531 aa, Artificial sequence
  Amino-acid sequence of PPO1 from wheat mutant (TaPPO1-B L418S/Y420I)
MAGATMATATVAAASPLRGRVTGRPHRVRPRCATASSATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLSNIIGGSTNTGIVSKTESDLVGAVDRDLRKMLINPRAADPLALGVRVWPQAIPQFLIGHLDRLAAAKSALGQGGYDGLFLGGNYVAGVALGRCIEGAYESASQVSDFLTKYAYK

SEQ ID NO: 40 - protein, 531 aa, Artificial sequence
  Amino-acid sequence of PPO1 form wheat mutant (TaPPO1-D L418S/Y420I)
MAGATMATATVTAASPLRGRVTGRPRRVRPRCAAASGATETPAAPGVRLSADCVIVGAGISGLCTAQALATRYGVGDLLITEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLSNIIGGSTNTGIVSKTESDLVEAVDSDLRKMLINPRAADPLALGVRVWPQAIPQFLIGHLDRLAAAKSALGRGGYDGLFLGGNYVAGVALGRCIEGAYESASQVSDFLTKYAYK

SEQ ID NO: 41 - protein, 558 aa, Artificial sequence
  Amino-acid sequence of PPO1 form tomato mutant (SlPPO1 L445S/Y447I)
MTTTAVVNHPSIFTHRSPLPSPSSSSSSSPSFLFLNRTNFIPYFSTSKRSSVNCNGWRTRCSVAKNYTVPPSEVDGNQLPELDCVVVGAGISGLCIAKVISANYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIESFCSGVYAGDPSKLSMKAAFGKVWKLEQTGGSIIGGTFKAIKERSSNPKPPRDPRLPTPKGQTVGSFRKGLRMLPDAICERLGSKVKLSWKLSSITKSDKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLSNIIGGATNTEIVSKTESQLVEAVDRDLRKMLIKPKAQDPFVTGVRVWPQAIPQFLVGHLDTLGTAKAALSDNGLDGLFLGGNYVSGVALGRCVEGAYEIASEVTGFLSQYAYK

SEQ ID NO: 42 - protein, 557 aa, Artificial sequence
  Amino-acid sequence of PPO1 form potato mutant (StPPO1 L444S/Y446I)
MTTTAVANHPSIFTHRSPLLSPSSSSSSPSFLFLNRTNFIPYFSTSKRNSVNCNGWRTRCSVAKDYTVPPSEVDGNQFPELDCVVVGAGISGLCITKVISDNYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIEPFCSGVYAGDPSKLSMKAAFGKVWKLEQTGGSIIGGTFKAIKERSSNPKPPRDPRLPTPKGQTVGSFRKGLRMLPDAICERLGSKVKLSWKLSSITKSEKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLSNIIGGATNTEIVSKTESQLVEAVDRDLRKMLIKPKAQDPFVTGVRVWPQAIPQFLVGHLDTLGTAKTALSDNGHDGLFLGGNYVSGVALGRCVEGAYEIASEVTGFLSQYAYK

SEQ ID NO: 43 - protein, 553 aa, Artificial sequence
  Amino-acid sequence of PPO1 from tobacco mutant (NtPPO1 L440S/Y442I)
MTTTPIANHPNVFTHRSPPSSSSSSPSAFLTRTSFLPFSSICKRNSVNCTGWRTRCSVAKDYTVPSSAVDGGPAAELDCVIVGAGISGLCIAQVMSANYPNLMVTEARDRAGGNITTVERDGYLWEEGPNSFQPSDPMLTMAVDCGLKDDLVLGDPNAPRFVLWKGKLRPVPSKLTDLPFFDLMSIPGKLRAGFGAIGLRPSPPGHEESVEQFVRRNLGGEVFERLIEPFCSGVYAGDPSKLSMKAAFGKVWKLEETGGSIIGGTFKAIKERSSTPKAPRDPRLPKPKGQTVGSFRKGLRMLPDAISARLGSKLKLSWKLSSITKSEKGGYRLTYETPEGVVSLQSRSIVMTVPSYVASNILRPLSVAAADALSNFYYPPVGAVTISYPQEAIRDERLVDGELKGFGQLHPRTQGVETLGTIYSSSLFPNRAPKGRVLLSNIIGGAKNPEILSKTESQLVEVVDRDLRKMLIKPKAQDPLVVGVRVWPQAIPQFLVGHLDTLSTAKAAMSDNGLEGLFLGGNYVSGVALGRCVEGAYEVASEVTGFLPRYAYK

SEQ ID NO: 44 - protein, 536 aa, Artificial sequence
  Amino-acid sequence of PPO1 form Arabidopsis thaliana mutant (AtPPO1 L423S/Y425I)
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLSNIIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 45 - protein, 539 aa, Artificial sequence
  Amino-acid sequence of PPO1 from upland cotton mutant (GhPPO1 L426S/Y428I)
MTALIDLSLLRSSPSVSPFSIPHHQHPPRFRKPFKLRCSLAEGPTISSSKIDGGESSIADCVIVGGGISGLCIAQALATKHRDVASNVIVTEARDRVGGNITTVERDGYLWEEGPNSFQPSDPILTMAVDSGLKDDLVLGDPNAPRFVLWEGKLRPVPSKPTDLPFFDLMSIAGKLRAGFGAIGIRPPPPGYEESVEEFVRRNLGAEVFERFIEPFCSGVYAGDPSKLSMKAAFGRVWKLEEIGGSIIGGTFKTIQERNKTPKPPRDPRLPKPKGQTVGSFRKGLTMLPEAIANSLGSNVKLSWKLSSITKLGNGGYNLTFETPEGMVSLQSRSVVMTIPSHVASNLLHPLSAAAADALSQFYYPPVASVTVSYPKEAIRKECLIDGELKGFGQLHPRSQGIETLGTIYSSSLFPNRAPSGRVLLSNIIGGATNTGILSKTEGELVEAVDRDLRKMLINPNAKDPLVLGVRVWPKAIPQFLVGHLDLLDSAKMALRDSGFHGLFLGGNYVSGVALGRCVEGAYEVAAEVKEFLSQYAYK

SEQ ID NO: 46 - protein, 538 aa, Artificial sequence
  Amino-acid sequence of PPO1 from radish mutant (RsPPO1 L425S/Y427I)
MDLSLLRPQPFLSPFSNPFPRSRPYYKPLNLHCSVSGGSVVGSSTIEGGGGGGKTVAADCVIVGGGISGLCIAQALVTKHPDAAKNVIVTEAKDRVGGNIITREEQGFLWEEGPNSFQPSDPMLTMVVDSGLKDDLVLGDPTAPRFVLWNGKLRPVPSKLTDLPFFDLMSIRGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPEAISARLGNKVKVSWKLSSITKLANGEYSLTYETPEGIVTVQSKSVVMTVPSHVASSLLRPLSDSAAEALSKLYYPPVAAVSISYPKEAIRSECLIDGELKGFGQLHPRTQKVETLGTIYSSSLFPNRAPPGRVLLSNIIGGTTNTGILSKSEGELVEAVDRDLRKMLIKPSSTDPLVLGVRVWPQAIPQFLIGHIDLVDAAKASLSSSGHEGIFLGGNYVAGVALGRCVECAYETATQVNDFMSRYTYK

SEQ ID NO: 47 - protein, 535 aa, Artificial sequence
  Amino-acid sequence of PPO1 from foxtail millet mutant (SiPPO1 L422S/Y424I)
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLSNIIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 48 - protein, 537 aa, Artificial sequence
  Amino-acid sequence of PPO1 from cabbage mutant (BoPPO1 L424S/Y426I)
MDLSLLRPQPFLSPFSNPFPRSRPYKPPNLRCSVSGGSVVGGSSTIEGGGGGKTVAADCVIVGGGISGLCIAQALVTKHPDAAKSVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQARNKAPKTTRDPRLPKPKGQTVGSFRKGLTMLPEAISARLGDKVKVSWKLSSISKLPSGGYSLTYETPEGIVTVQSKSVVMTVPSHVASSLLRPLSDSAAEALSKLYYPPVAAVSISYLKEAIRSECLIDGELKGFGQLHPRTQKVETLGTIYSSSLFPNRAPPGRVLLSNIIGGATNTGILSKSEGELVEAVDRDLRKMLIMPSSTDPLVLGVKVWPQAIPQFLIGHIDLVDAAKASLSSSGHEGLFLGGNYVAGVALGRCVEGAYETATQVNDFMSRYAYK

SEQ ID NO: 49 - DNA, 1258 bp, Artificial sequence
  Amino-acid sequence of PPO1 from homologous replacement repair template of rice mutant (OsPPO1 L423S/Y425I)
acatgaactagtaatgattggggtgcttaaccatttgttctgcatttcctccattttcaggttgggtagcaaagtcaaactttcatggaagttgacaagcattacaaagtcagacaacaaaggatatgcattagtgtatgaaacaccagaaggggtggtctcggtgcaagctaaaactgttgtcatgaccatcccatcatatgttgctagtgatatcttgcggccactttcagtaagttatatatatttaattaactttctgttcccaaaatacactgcagcacttcattgcttcctgaggtcctcgattcatttttcggtagacaggaagtagtattcatttgcactttttaagggattaattcaacatatccactggaaatatacatatcctacacatcctgtcaacatacttgctaaacagcattttgtttgagttgactggcatctcagcagccaattactatctttaggggacaagccacattcttaataaatcctgtcggaaatcactttttgatttttatagattagtgttgtcatagaatttggcttggtgatctacttggtaaggttaactgattcacaagtcggacaatttcttcaccaatctagcagtatgtttaagtgtgttggtacttaattctaaatgtcctgcgcatggtaacatatcatatgcaaaaattcctcagtaaccgaaatttatactgtaagttttaactgtctttacactgttaattttagacatacttcttccttgcttgttcattgaacttgtttcccccttccacagagtgatgcagcagatgctctgtcaatattctattatccaccagttgctgctgtaactgtttcatatccaaaagaagcaattagaaaagaatgcttaattgacggagagctccagggtttcggccagctgcatccgcgtagtcagggagttgagactttaggtacttataggaattcaaccttattattcttctaacatataaatgaactaatctttcttgtctagtttgcatttattgtggattaagtttggttatattgttcttacaagtttgtggtattattttgtataggaacaatatatagctcatcactctttccaaatcgtgctccagctggaagggtgttactttccaacatcataggaggttctacaaatacagggattgtttccaaggtatcgctgtcaagttgtttattttgcgactatatgattacagtatcctgtttttcaactccagctgct

(19) United States
(12) Patent Application Publication
(10) Pub. No.: US 2024/0368618 A1
(43) Pub. Date: Nov. 7, 2024

(54) PPO POLYPEPTIDES TOLERANT TO PPO-INHIBITING HERBICIDES AND USE THEREOF

(71) Applicant: QINGDAO KINGAGROOT CHEMICAL COMPOUND CO., LTD., QINGDAO, SHANDONG (CN)

(72) Inventors: Sudong MO, QINGDAO, SHANDONG (CN); Guizhi LIU, QINGDAO, SHANDONG (CN); Lei WANG, QINGDAO, SHANDONG (CN); Qiqi HOU, QINGDAO, SHANDONG (CN); Bo CHEN, QINGDAO, SHANDONG (CN)

(21) Appl. No.: 18/284,662

(22) PCT Filed: Mar. 25, 2022
(86) PCT No.: PCT/CN2022/082947

§ 371 Date: Sep. 28, 2023

(30) Foreign Application Priority Data
Apr. 2, 2021 (CN) 202110361141.6
Feb. 15, 2022 (CN) 202210136187.2

(51) Int. Cl. C12N 15/82; C12N 9/02; C12N 9/22; C12N 15/11

(52) U.S. Cl. CPC C12N 15/8274; C12N 9/001; C12N 9/22; C12N 15/11; C12N 15/8213; C12Y 103/03004; C12N 2310/20

(57) ABSTRACT

The present invention relates to the field of biotechnology, and specifically relates to a PPO polypeptide tolerant to PPO-inhibiting herbicides and use thereof. The said polypeptide contains the motif “LLLNYI”, wherein leucine L at position 3 in the said motif is substituted with any other amino acid, or tyrosine Y at position 5 is substituted with any other amino acid. It can be used in plants, including commercial crops, to greatly improve plant resistance to PPO-inhibiting herbicides according to the herbicide resistance characteristics and herbicide selectivity, so as to control weed growth economically.

### TECHNICAL FIELD

The present invention relates to the field of biotechnology, and specifically relates to PPO polypeptides tolerant to PPO-inhibiting herbicides and use thereof.

### BACKGROUND OF THE INVENTION

Weeds are one of the key factors affecting crop yield in agricultural production. Herbicides are the main technical means for weed control. Herbicide modes of action are classified into 28 categories by Weed Science Society of America (weedscience.org) according to the different target sites of herbicides in plants, wherein Group 14 (Group 14; HRAC GROUP E) is the Protoporphyrinogen IX oxidase (PPO) inhibitor (http://www.weedscience.org/).

Protoporphyrinogen IX oxidase (PPOX, PPX or PPO; EC 1.3.3.4) is the last common enzyme in the synthetic pathway of chlorophyll and heme. Protoporphyrinogen IX is converted into Protoporphyrin IX catalyzed by PPO in the presence of oxygen molecules.

PPO is an important herbicide target site in plants which inhibits proporphyrinogen oxidase in plants and results in the intracellular accumulation of the substrate protoporphyrinogen that catalyses the reaction. The protoporphyrinogen accumulation in chloroplast and mitochondrion in cells leads to non-enzymatic oxidation of proporphyrinogen by 02. Under light conditions, nonenzymatic oxidation produces singlet oxygen. Singlet oxygen results in the lipid oxidation in the endomembrane system, then brings about the oxidative damage of these endomembrane systems, thereby killing plant cells (Future Med Chem. 2014 April; 6(6): 597-599. doi: 10.4155/fmc.14.29).

The evolutionary relationship of PPO enzymes in the organism kingdom is studied by researching their sequence similarities, and PPO enzymes are divided into three categories: HemG, HemJ, and HemY. In most cases, a single species possesses only one of the categories. Among them, HemG is generally distributed in γ-proteobacteria, HemJ is distributed in α-proteobacteria and transferred into other proteobacterias and cyanobacteria, while HemY is the only PPO enzyme in eukaryotes (Genome Biol Evol. 2014 August; 6(8):2141-55. doi: 10.1093/gbe/evu170).

PPO genes have been identified from certain organisms. For example, these genes known by us include the PPO1 gene (Genbank ID Y13465) and PPO2 gene (Genbank ID Y13466) of Nicotiana tabacum, the PPO gene of Arabidopsis thaliana (Genbank ID D83139), the HemY gene of Bacillus subtilis (Genbank ID M97208), the PPO gene of mice (Genbank ID D45185), the PPO gene of human beings (Genbank ID D38537), the PPO gene of Saccharomyces cerevisiae (Genbank ID Z71381), the hemG gene of Escherichia coli (Genbank ID X68660), etc.

Generally, there are at least two kinds of PPO genes in plant, named as PPO1 and PPO2, respectively, wherein PPO1 is generally located in the chloroplast of plants while PPO2 is in the mitochondrion of plant cells. However, the mRNA of the PPO2 gene in certain Amaranthaceae plants differ in translation initiation sites (TIS), thereby produces PPO2 polypeptides of different lengths. For example, the PPO2 gene in spinach (Spinacia oleracea L) expresses two PPO2 proteins with the molecular weight of 58 KD and 56 KD, respectively, and the two proteins have the difference of 26 polypeptides in length. Wherein, the longer one is located in chloroplasts and the shorter one in mitochondria (J Biol Chem. 2001 Jun. 8; 276(23):20474-81. doi: 10.1074/jbc.M101140200. Epub 2001 Mar. 23).

When PPO activity is inhibited by a certain compound, the production of chlorophyll and heme will also be inhibited. The substrate protoporphyrinogen IX will be separated from the normal porphyrin biosynthetic pathway, rapidly separating from chloroplast and entering into cytoplasm. The protoporphyrinogen IX is oxidized to protoporphyrin IX and accumulates on cell membrane. The accumulated protoporphyrin IX produces high active singlet oxygens (102) with the action of light and oxygen molecules and causes destruction of cell membrane, leading to rapid death of plant cells. Due to the use of PPO-inhibiting herbicides, cases of weeds resistant to certain PPO-inhibiting herbicides have been reported. (Pest Manag Sci. 2014 September; 70(9):1358-66. doi: 10.1002/ps.3728. Epub 2014 Feb. 24).

For example, in tall waterhemp (Amaranthus tuberculatus), resistance to herbicide lactofen is conferred by the deletion of glycine (AG210) at position 210 of the PPO2L gene (Proc Natl Acad Sci USA. 2006 Aug. 15; 103(33):12329-34. doi: 10.1073/pnas.0603137103. Epub 2006 Aug. 7).

In palmer amaranth (Amaranthus palmeri), resistance to herbicide fomesafen is conferred by the mutation of arginine to glycine or methionine at position 98 of the PPO2 gene (R98G, R98M) (Pest Manag Sci. 2017 August; 73(8):1559-1563. doi: 10.1002/ps.4581. Epub 2017 May 16).

In palmer amaranth (Amaranthus palmeri), resistance to fomesafen is conferred by the mutation of glycine to alanine (G399A) at position 399 of the PPO2 gene (Front Plant Sci. 2019 May 15; 10:568. doi: 10.3389/fpls.2019.00568. eCollection 2019).

In ragweed (Ambrosia artemisiifolia), resistance to flumioxazin is conferred by the mutation of arginine to leucine at position 98 of the PPO2 gene (R98L) (Weed Science, 60(3):335-344 (2012)).

In goosegrass (Eleusine indica), resistance to oxadiazon is conferred by the mutation of alanine to threonine at position 212 of the PPO1 gene (A212T) (Pest Manag Sci. 2020 May; 76(5):1786-1794. doi: 10.1002/ps.5703. Epub 2020 Jan. 23).

### BRIEF SUMMARY OF THE INVENTION

The present invention relates to a PPO polypeptide or a bioactive fragment thereof tolerant to a PPO-inhibiting herbicide.

The present invention also relates to an isolated polynucleotide and the corresponding plant genome, vector construct, or host cell.

In the other aspect, the present invention provides a producing method of a plant cell or plant to gain or improve its tolerance to a PPO-inhibiting herbicide, and a plant produced by the method.

In another aspect, the present invention provides a method of enabling a plant to gain or improve tolerance to a PPO-inhibiting herbicide.

The present invention also provides a method of gaining or improving the tolerance of a plant cell, plant tissue, plant part or plant to a PPO-inhibiting herbicide.

The present invention further relates to use of the PPO polypeptide or a bioactive fragment thereof or the polynucleotide for gaining or improving tolerance of a host cell, plant cell, plant issue, plant part or plant to a PPO-inhibiting herbicide.

The present invention further relates to a method for controlling weeds in a plant cultivation site.

### BRIEF DESCRIPTION OF THE SEVERAL VIEWS OF THE DRAWING(S)

FIG. 1 shows the alignment of PPO amino acid sequences from different plants, wherein the marked box indicates the conserved amino acid motifs at the screening sites, representing successively from top to bottom: rice (Oryza sativa L.), corn (Zea mays), Arabidopsis thaliana, soybean (Glycine max), tobacco (Nicotiana tabacum), sorghum (Sorghum bicolor), tomato (Solanum lycopersicum), barley (Hordeum vulgare), oilseed rape (Brassica napus), peanut (Arachis hypogaea), wheat (Triticum aestivum), cabbage (Brassica oleracea), foxtail millet (Setaria italica), radish (Raphanus sativus), potato (Solanum tuberosum), upland cotton (Gossypium hirsutum), yam (Dioscorea cayenensis), cassava (Manihot esculenta), pepper (Capsicum annuum), squash (Cucurbita moschata), cucumber (Cucumis sativus), lettuce (Lactuca sativa), sesame (Sesamum indicum), sunflower (Helianthus annuus), mulberry (Morus alba), cowpea (Vigna unguiculata), strawberry (Fragaria ananassa), apple (Malus domestica), peach (Prunus persica), cherry (Prunus pseudocerasus), apricot (Prunus armeniaca), grape vine (Vitis vinfera), papaya (Carica papaya), alfalfa (Medicago sativa).

FIG. 2 shows the cell growth level of PPO-deficient Escherichia coli (ΔhemG) treated with compound A at 0 nM, 100 nM, 300 nM and 1000 nM (nanomloar) after transformed by pET44a empty vector and rice OsPPO1 wild-type gene (WT).

FIG. 3 shows the cell growth level of PPO-deficient Escherichia coli (ΔhemG) transformant transformed by OsPPO1 wild-type gene (indicated as WT) or various OsPPO1 mutant genes when treated with compound A at concentrations of 0 nM and 500 nM.

FIG. 4 shows the cell growth level of PPO-deficient Escherichia coli (ΔhemG) transformant transformed by OsPPO1 wild-type gene (indicated as WT) or various OsPPO1 mutant genes when treated with compound A at concentrations of 0 μM, 1 μM, 10 μM, 20 μM, 50 μM and 100 μM, respectively.

FIG. 5 shows the cell growth level of PPO-deficient Escherichia coli (ΔhemG) transformant transformed by ZmPPO1 wild-type gene (indicated as ZmPPO1-WT) or various ZmPPO1 mutant genes when treated with compound A at concentrations of 0 μM, 5 μM, 50 μM and 100 μM, respectively.

FIG. 6 shows the resistance test of other crop resistance site combinations against different PPO-inhibiting herbicides such as compound A, saflufenacil and flumioxazin.

FIG. 7 shows the resistance test of other crop resistance site combinations against different PPO-inhibiting herbicides such as epyrifenacil, sulfentrazone and tiafenacil.

FIG. 8 shows the resistance test of other crop resistance site combinations against different PPO-inhibiting herbicides such as fomesafen and trifludimoxazin.

FIG. 9 shows the cell growth level of PPO-deficient Escherichia coli (ΔhemG) transformant transformed by OsPPO1 wild-type gene (indicated as WT) or various OsPPO1 mutant genes when treated with 100 nM flumioxazin, 100 nM oxyfluorfen, 500 nM saflufenacil, 5 μM pyraclonil, 1 μM carfentrazone-ethyl and 10 μM fomesafen, respectively.

FIG. 10 shows mutant enzyme activity determination. Differences between enzymatic kinetics curves of the rice OsPPO1 wild-type (indicated as WT), Y425I , and L423S/Y425I are shown as well. The enzyme activity L423S/Y425 mutant is higher than WT, while the enzyme activity Y425I is lower than WT.

FIG. 11 shows the growth performance of rice seedling strains with homologous replacement at L423S/Y425I site when treated with compound A at the rate of 9 g/ha in comparison with wild-type strains.

FIG. 12 shows the overexpressed soybean PPO1 WT and L430S/Y432I Arabidopsis thaliana seed treated with different concentrations of compound A. Compared with wild-type Arabidopsis thaliana, both the overexpressed soybean PPO1 WT and L430S/Y432I show certain level of tolerance to compound A in Arabidopsis thaliana, but the tolerance of the overexpressed L430S/Y432I to compound A is much higher than that of the overexpressed wild-type. Wherein, wild-type indicates wild-type Arabidopsis thaliana; pHSE-GmPPO1 WT indicates overexpressed soybean PPO1; pHSE-GmPPO1 L430S/Y432I indicates overexpressed soybean PPO1 L430S/Y432I.

FIG. 13 shows the overexpressed oilseed rape PPO1 WT and L424S/Y426I Arabidopsis thaliana seed treated with different concentrations of compound A. Compared with wild-type Arabidopsis thaliana, both the overexpressed oilseed rape PPO1 WT and L424S/Y426I show certain level of tolerance to compound A in Arabidopsis thaliana, but the tolerance of the overexpressed L424S/Y426I to compound A is much higher than that of the overexpressed wild-type. Wherein, wild-type indicates wild-type Arabidopsis thaliana; pHSE-BnPPO1-C5 WT indicates overexpressed oilseed rape PPO1; pHSE-BnPPO1-C5L424S/Y426I indicates overexpressed oilseed rape PPO1 L424S/Y426I.

FIG. 14 shows the overexpressed corn PPO1 WT and L424W/Y426L Arabidopsis thaliana seed treated with different concentrations of compound A. Compared with wild-type Arabidopsis thaliana, both the overexpressed corn PPO1 WT and L424W/Y426L show certain level of tolerance to compound A in Arabidopsis thaliana, but the tolerance of the overexpressed L424W/Y426 μL to compound A is much higher than that of the overexpressed wild-type. Wherein, wild-type indicates wild-type Arabidopsis thaliana; pHSE-ZmPPO1 WT indicates overexpressed corn PPO1; pHSE-ZmPPO1 L424W/Y426 L indicates overexpressed corn PPO1 L424W/Y426L.

FIG. 15 shows the overexpressed rice PPO1 WT and L423S/Y425I Arabidopsis thaliana seed treated with different concentrations of compound A. Compared with wild-type Arabidopsis thaliana, both the overexpressed rice PPO1 WT and L423S/Y425I show certain level of tolerance to compound A in Arabidopsis thaliana, but the tolerance of the overexpressed L423S/Y425I to compound A is much higher than that of the overexpressed wild-type. Wherein, wild-type indicates wild-type Arabidopsis thaliana; pHSE-OsPPO1 WT indicates overexpressed rice PPO1; pHSE-OsPPO1 L423S/Y425I indicates overexpressed rice PPO1 L423S/Y425I.

FIG. 16 shows the overexpressed rice PPO1 WT and L423S/Y425I Arabidopsis thaliana seed treated with different concentrations of flumioxazin. Compared with wild-type Arabidopsis thaliana, both the overexpressed rice PPO1 WT and L423S/Y425I show certain level of tolerance to flumioxazin in Arabidopsis thaliana, but the flumioxazin tolerance of the overexpressed L423S/Y425I is much higher than that of the overexpressed wild-type. Wherein, wild-type indicates wild-type Arabidopsis thaliana; pHSE-OsPPO1 WT indicates overexpressed rice PPO1; pHSE-OsPPO1 L423S/Y425I indicates overexpressed rice PPO1 L423S/Y425I.

FIG. 17 shows the overexpressed rice PPO1 WT and L423S/Y425I Arabidopsis thaliana seed treated with different concentrations of saflufenacil. Compared with the wild type Arabidopsis thaliana, both the overexpressed rice PPO1 WT and L423S/Y425I show certain level of tolerance to saflufenacil in Arabidopsis thaliana, but the saflufenacil tolerance of overexpressed L423S/Y425I is much higher than that of the overexpressed wild type. Wherein, wild-type indicates wild-type Arabidopsis thaliana; pHSE-OsPPO1 WT indicates overexpressed rice PPO1; pHSE-OsPPO1 L423S/Y425I indicates overexpressed rice PPO1 L423S/Y425I.

FIG. 18 shows the overexpressed soybean PPO1 WT and L430S/Y432I Arabidopsis thaliana seed treated with different concentrations of flumioxazin. Compared with the wild type Arabidopsis thaliana, both the overexpressed soybean PPO1 WT and L430S/Y432I show certain level of tolerance to flumioxazin in Arabidopsis thaliana, but the flumioxazin tolerance of overexpressed L430S/Y432I is much higher than that of the overexpressed wild type. Wherein, wild-type indicates wild-type Arabidopsis thaliana; pHSE-GmPPO1 WT indicates overexpressed soybean PPO1; pHSE-GmPPO1 L430S/Y432I indicates overexpressed soybean PPO1 L430S/Y432I.

FIG. 19 shows the overexpressed soybean PPO1 WT and L430S/Y432I Arabidopsis thaliana seed treated with different concentrations of saflufenacil. Compared with the wild type Arabidopsis thaliana, both the overexpressed soybean PPO1 WT and L430S/Y432I show certain level of tolerance to saflufenacil in Arabidopsis thaliana, but the saflufenacil tolerance of overexpressed L430S/Y432I is much higher than that of the overexpressed wild type. Wherein, wild-type indicates wild-type Arabidopsis thaliana; pHSE-GmPPO1 WT indicates overexpressed soybean PPO1; pHSE-GmPPO1 L430S/Y432I indicates overexpressed soybean PPO1 L430S/Y432I.

FIG. 20 shows the overexpressed corn PPO1 WT and L424W/Y426 L Arabidopsis thaliana seed treated with different concentrations of flumioxazin. Compared with the wild type Arabidopsis thaliana, both the overexpressed corn PPO1 WT and L424W/Y426 L show certain level of tolerance to flumioxazin in Arabidopsis thaliana, but the flumioxazin tolerance of overexpressed L424W/Y426 L is much higher than that of the overexpressed wild type. Wherein, wild-type indicates wild-type Arabidopsis thaliana; pHSE-ZmPPO1 WT indicates overexpressed corn PPO1; pHSE-ZmPPO1 L424W/Y426 L indicates overexpressed corn PPO1 L424W/Y426L.

FIG. 21 shows the overexpressed corn PPO1 WT and L424W/Y426 L Arabidopsis thaliana seed treated with different concentrations of saflufenacil. Compared with the wild type Arabidopsis thaliana, both the overexpressed corn PPO1 WT and L424W/Y426 L show certain level of tolerance to saflufenacil in Arabidopsis thaliana, but the saflufenacil tolerance of overexpressed L424W/Y426 L is much higher than that of the overexpressed wild type. Wherein, wild-type indicates wild-type Arabidopsis thaliana; pHSE-ZmPPO1 WT indicates overexpressed corn PPO1; pHSE-ZmPPO1 WTL424W/Y426 L indicates overexpressed corn PPO1 L424W/Y426L.

FIG. 22 shows the overexpressed oilseed rape PPO1 WT and L424S/Y426I Arabidopsis thaliana seed treated with different concentrations of flumioxazin. Compared with the wild type Arabidopsis thaliana, both the overexpressed oilseed rape PPO1 WT and L424S/Y426I show certain level of tolerance to flumioxazin in Arabidopsis thaliana, but the flumioxazin tolerance of overexpressed L424S/Y426I is much higher than that of the overexpressed wild type. Wherein, wild-type indicates wild-type Arabidopsis thaliana; pHSE-BnPPO1-C5 WT indicates overexpressed oilseed rape PPO1; pHSE-BnPPO1-C5 L424S/Y426I indicates overexpressed oilseed rape PPO1 L424S/Y426I.

FIG. 23 shows the overexpressed oilseed rape PPO1 WT and L424S/Y426I Arabidopsis thaliana seed treated with different concentrations of saflufenacil. Compared with the wild type Arabidopsis thaliana, both the overexpressed oilseed rape PPO1 WT and L424S/Y426I show certain level of tolerance to saflufenacil in Arabidopsis thaliana, but the saflufenacil tolerance of overexpressed L424S/Y426I is much higher than that of the overexpressed wild type. Wherein, wild-type indicates wild-type Arabidopsis thaliana; pHSE-BnPPO1-C5 WT indicates overexpressed oilseed rape PPO1; pHSE-BnPPO1-C5 L424S/Y426I indicates overexpressed oilseed rape PPO1 L424S/Y426I.

FIG. 24 shows the test result of overexpressed rice PPO1 WT and L423S/Y425I rice seedlings sprayed with different concentrations of compound A. Wherein, WT indicates Huaidao No. 5 wild-type; MT1 and MT2 indicate overexpressed rice PPO1 WT; MT3 and MT4 indicate overexpressed rice PPO1 L423S/Y425I.

| SEQ ID NO | Name |
|---|---|
| SEQ ID NO: 1 | Amino-acid sequence of PPO1 from wild-type rice |
| | (OsPPO1 WT) |
| SEQ ID NO: 2 | Amino-acid sequence of PPO1 from wild-type corn |
| | (ZmPPO1 WT) |
| SEQ ID NO: 3 | Amino-acid sequence of PPO1 wild-type from oilseed |
| | rape (BnPPO1-C5) |
| SEQ ID NO: 4 | Amino-acid sequence of PPO1 from wild-type oilseed |
| | rape (BnPPO1-A10) |
| SEQ ID NO: 5 | Amino-acid sequence of PPO1 from wild-type peanut |
| | (AhPPO1-A) |
| SEQ ID NO: 6 | Amino-acid sequence of PPO1 from wild-type peanut |
| | (AhPPO1-B) |
| SEQ ID NO: 7 | Amino-acid sequence of PPO1 from wild-type |
| | soybean (GmPPO1) |
| SEQ ID NO: 8 | Amino-acid sequence of PPO1 from wild-type |
| | sorghum (SbPPO1) |
| SEQ ID NO: 9 | Amino-acid sequence of PPO1 from wild-type wheat |
| | (TaPPO1-A) |
| SEQ ID NO: 10 | Amino-acid sequence of PPO1 from wild-type wheat |
| | (TaPPO1-B) |
| SEQ ID NO: 11 | Amino-acid sequence of PPO1 from wild-type wheat |
| | (TaPPO1-D) |
| SEQ ID NO: 12 | Amino-acid sequence of PPO1 form wild-type tomato |
| | (SlPPO1) |
| SEQ ID NO: 13 | Amino-acid sequence of PPO1 from wild-type potato |
| | (StPPO1) |
| SEQ ID NO: 14 | Amino-acid sequence of PPO1 form wild-type tobacco |
| | (NtPPO1) |
| SEQ ID NO: 15 | Amino-acid sequence of PPO1 from wild-type |
| | Arabidopsis thaliana (AtPPO1) |
| SEQ ID NO: 16 | Amino-acid sequence of PPO1 from wild-type upland |
| | cotton (GhPPO1) |
| SEQ ID NO: 17 | Amino-acid sequence of PPO1 from wild-type radish |
| | (RsPPO1) |
| SEQ ID NO: 18 | Amino-acid sequence of PPO1 from wild-type foxtail |
| | millet (SiPPO1) |
| SEQ ID NO: 19 | Amino-acid sequence of PPO1 form wild-type cabbage |
| | (BoPPO1) |
| SEQ ID NO: 20 | Amino-acid sequence of PPO1 from rice mutant |
| | (OsPPO1 L423S) |
| SEQ ID NO: 21 | Amino-acid sequence of PPO1 from rice mutant |
| | (OsPPO1 L423I) |
| SEQ ID NO: 22 | Amino-acid sequence of PPO1 from rice mutant |
| | (OsPPO1 L423G) |
| SEQ ID NO: 23 | Amino-acid sequence of PPO1 from rice mutant |
| | (OsPPO1 Y425M) |
| SEQ ID NO: 24 | Amino-acid sequence of PPO1 from rice mutant |
| | (OsPPO1 Y425I) |
| SEQ ID NO: 25 | Amino-acid sequence of PPO1 from rice mutant |
| | (OsPPO1 Y425V) |
| SEQ ID NO: 26 | Amino-acid sequence of PPO1 from rice mutant |
| | (OsPPO1 L423S/Y425I) |
| SEQ ID NO: 27 | Amino-acid sequence of PPO1 from corn mutant |
| | (ZmPPO1 L424T/Y426V) |
| SEQ ID NO: 28 | Amino-acid sequence of PPO1 form corn mutant |
| | (ZmPPO1 L424S/Y426V) |
| SEQ ID NO: 29 | Amino-acid sequence of PPO1 from corn mutant |
| | (ZmPPO1 L424V/Y426L) |
| SEQ ID NO: 30 | Amino-acid sequence of PPO1 from corn mutant |
| | (ZmPPO1 L424W/Y426L) |
| SEQ ID NO: 31 | Amino-acid sequence of PPO1 form corn mutant |
| | (ZmPPO1 L424S/Y426I) |
| SEQ ID NO: 32 | Amino-acid sequence of PPO1 from oilseed rape |
| | mutant (BnPPO1-C5 L424S/Y426I) |
| SEQ ID NO: 33 | Amino-acid sequence of PPO1 from oilseed rape |
| | mutant (BnPPO1-A10 L423S/Y425I) |
| SEQ ID NO: 34 | Amino-acid sequence of PPO1 from peanut mutant |
| | (AhPPO1-A L445S/Y447I) |
| SEQ ID NO: 35 | Amino-acid sequence of PPO1 from peanut mutant |
| | (AhPPO1-B L439S/Y441I) |
| SEQ ID NO: 36 | Amino-acid sequence of PPO1 from soybean mutant |
| | (GmPPO1 L430S/Y432I) |
| SEQ ID NO: 37 | Amino-acid sequence of PPO1 from sorghum mutant |
| | (SbPPO1 L423S/Y425I) |
| SEQ ID NO: 38 | Amino-acid sequence of PPO1 form wheat mutant |
| | (TaPPO1-A L418S/Y420I) |
| SEQ ID NO: 39 | Amino-acid sequence of PPO1 from wheat mutant |
| | (TaPPO1-B L418S/Y420I) |
| SEQ ID NO: 40 | Amino-acid sequence of PPO1 form wheat mutant |
| | (TaPPO1-D L418S/Y420I) |
| SEQ ID NO: 41 | Amino-acid sequence of PPO1 form tomato mutant |
| | (SlPPO1 L445S/Y447I) |
| SEQ ID NO: 42 | Amino-acid sequence of PPO1 form potato mutant |
| | (StPPO1 L444S/Y446I) |
| SEQ ID NO: 43 | Amino-acid sequence of PPO1 from tobacco mutant |
| | (NtPPO1 L440S/Y442I) |
| SEQ ID NO: 44 | Amino-acid sequence of PPO1 form Arabidopsis |
| | thaliana mutant (AtPPO1 L423S/Y425I) |
| SEQ ID NO: 45 | Amino-acid sequence of PPO1 from upland cotton |
| | mutant (GhPPO1 L426S/Y428I) |
| SEQ ID NO: 46 | Amino-acid sequence of PPO1 from radish mutant |
| | (RsPPO1 L425S/Y427I) |
| SEQ ID NO: 47 | Amino-acid sequence of PPO1 from foxtail millet |
| | mutant (SiPPO1 L422S/Y424I) |
| SEQ ID NO: 48 | Amino-acid sequence of PPO1 from cabbage mutant |
| | (BoPPO1 L424S/Y426I) |
| SEQ ID NO: 49 | Amino-acid sequence of PPO1 from homologous |
| | replacement repair template of rice mutant |
| | (OsPPO1 L423S/Y425I) |

### DETAILED DESCRIPTION OF THE INVENTION

Some terms used in the specification are defined as follows.

In the present invention, the term “herbicide” refers to an active ingredient that can kill, control or otherwise adversely modifies the growth of plants. The term “herbicide tolerance” or “herbicide resistance” refers to a situation that a plant continues to grow even after the treatment of a herbicide which are capable of killing normal or wild-type plants or inhibiting growth thereof, or weakening or ceasing plant growth ability compared to wild-type plants. The above herbicide includes PPO-inhibiting herbicides, which can be divided into pyrimidinediones, diphenyl-ethers, phenylpyrazoles, N-phenylphthalimides, thiadiazoles, oxadiazoles, triazolinones, oxazolidinediones, and other herbicides with different chemical structures.

Generally, if the PPO-inhibiting herbicides and/or other herbicidal compounds, as described herein and available to be employed in the context of the present invention, are capable of forming geometrical isomers such as E/Z isomers, both of themselves, pure isomers and mixtures thereof may be used in the compositions according to the invention. If the PPO-inhibiting herbicides and/or other herbicidal compounds as described herein have one or more centers of chirality and, as a consequence, are present as enantiomers or diastereomers, both of themselves, pure isomers and mixtures thereof may be used in the compositions according to the invention. If the PPO-inhibiting herbicides and/or other herbicidal compounds as described herein have ionizable functional groups, they can also be used in the form of their agriculturally acceptable salts. Suitable are, in general, the salts of those cations and the acid addition salts of those acids whose cations and anions, respectively, have no adverse effects on the activity of the active compounds. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C1-C4-alkyl, hydroxy-C1-C4-alkyl, C1-C4-alkoxy-C1-C4-alkyl, hydroxy-C1-C4-alkoxy-C1-C4-alkyl, phenyl or benzyl, preferably ammonium, methyl-ammonium, isopropyl-ammonium, dimethyl-ammonium, diisopropyl-ammonium, trimethyl-ammonium, heptyl-ammonium, dodecyl-ammonium, tetradecyl-ammonium, tetramethyl-ammonium, tetraethyl-ammonium, tetrabutyl-ammonium, 2-hydroxyethyl-ammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium(trolamine salt), tris(2-hydroxypropyl)ammonium, benzyltrimethylammonium, benzyltriethylammonium and N,N,N-trimethylethanolammonium (choline salt), additionally phosphonium ions, sulfonium ions, preferably tri(C1-C4-alkyl)sulfonium such as trimethylsulfonium, and sulfoxonium ions, preferably tri(C1-C4-alkyl)sulfoxonium ions, and finally the salts of polybasic amines such as N,N-bis-(3-aminopropyl)methylamine and diethylenetriamine. Anions of useful acid addition salts are primarily irons of chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C1-C4-alkanoic acids, preferably formate, acetate, propionate and butyrate.

The PPO-inhibiting herbicides and/or other herbicidal compounds as described herein having a carboxyl group can be employed in the form of an acid, in the form of an agriculturally suitable salt as mentioned above or else in the form of an agriculturally acceptable derivative, for example as amides such as mono- and di-C1-C6-alkylamides or arylamides, as esters such as allylesters, propargyl esters, C1-C10-alkyl esters, alkoxyalkyl esters, tefuryl ((tetrahydrofuran-2-yl)methyl) esters and also as thioesters such as C1-C10-alkylthio esters. Preferred mono- and di-C1-C6-alkylamides are methyl and dimethylamides. Preferred arylamides are, for example, anilides and 2-chloroanilides. Preferred alkyl esters are, for example, methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C1-C4-alkoxy-C1-C4-alkyl esters are the straight-chain or branched C1-C4-alkoxy ethyl esters, for example the 2-methoxyethylester, 2-ethoxyethylester, 2-butoxyethyl (butotyl)ester, 2-butoxypropyl ester or 3-butoxypropyl ester. An example of a straight-chain or branched C1-C10-alkylthio ester is the ethylthio ester.

In an exemplary embodiment, the pyrimidinediones herbicides include but are not limited to, butafenacil (CAS NO: 134605-64-4), saflufenacil (CAS NO: 372137-35-4), benzfendizone (CAS NO:158755-95-4), tiafenacil (CAS NO: 1220411-29-9), [3-[2-Chloro-4-fluoro-5-(1-methyl-6-trifluoromethyl-2,4-dioxo-1,2,3,4-tetrahydropyrimidine-3-yl)phenoxy]-2-pyridyloxy]acetic acid ethyl ester (Epyrifenacil, CAS NO: 353292-31-6), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS NO: 1304113-05-0),3-[7-chloro-5-fluoro-2-(trifluoromethyl)-1H-benzimidazol-4-yl]-1-methyl-6-(trifluoromethyl)-1H-pyrimidine-2,4-dione (CAS NO: 212754-02-4), flupropacil (CAS NO: 120890-70-2), isoxazoline-containing uracils disclosed in CN105753853A (eg. compound

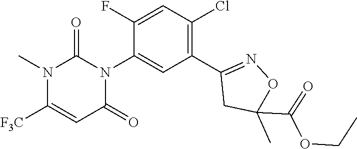 [SMILES: CCOC(=O)C1(C)CC(c2cc(-n3c(=O)cc(C(F)(F)F)n(C)c3=O)c(F)cc2Cl)=NO1]

uracil pyridines disclosed in WO2017/202768, and uracils disclosed in WO2018/019842.

The diphenyl-ethers herbicides include but are not limited to, fomesafen (CAS NO: 72178-02-0), oxyfluorfen (CAS NO: 42874-03-3), aclonifen (CAS NO:74070-46-5), lactofen (CAS NO: 77501-63-4), chlomethoxyfen (CAS NO: 32861-85-1), chlornitrofen (CAS NO: 1836-77-7), fluoroglycofen-ethyl (CAS NO: 77501-90-7), acifluorfen or sodium salt (CAS NO: 50594-66-6 or 62476-59-9), bifenox (CAS NO: 42576-02-3), ethoxyfen (CAS NO: 188634-90-4), ethoxyfen-ethyl (CAS NO: 131086-42-5), fluoronitrofen (CAS NO: 13738-63-1), furyloxyfen (CAS NO: 80020-41-3), nitrofluorfen (CAS NO: 42874-01-1) and halosafen (CAS NO: 77227-69-1).

The phenylpyrazoles herbicides include but are not limited to, pyraflufen-ethyl (CAS NO: 129630-19-9) and fluazolate (CAS NO: 174514-07-9).

The N-Phenyl-imides herbicides include but are not limited to, flumioxazin (CAS NO: 103361-09-7), cinidon-ethyl (CAS NO: 142891-20-1), flumipropyn (CAS NO: 84478-52-4) and flumiclorac-pentyl (CAS NO: 87546-18-7).

The thiadiazoles herbicides include but are not limited to, fluthiacet-methyl (CAS NO: 117337-19-6), fluthiacet (CAS NO: 149253-65-6) and thidiazimin (CAS NO: 123249-43-4).

The oxadiazoles herbicides include but are not limited to, oxadiargyl (CAS NO: 39807-15-3) and oxadiazon (CAS NO: 19666-30-9).

The triazolinones herbicides include but are not limited to, carfentrazone (CAS NO: 128621-72-7), carfentrazone-ethyl (CAS NO: 128639-02-1), sulfentrazone (CAS NO: 122836-35-5), azafenidin (CAS NO: 68049-83-2) and bencarbazone (CAS NO: 173980-17-1).

The oxazolidinediones herbicides include but are not limited to, pentoxazone (CAS NO: 110956-75-7).

Other herbicides include but are not limited to, pyraclonil (CAS NO: 158353-15-2), flufenpyr-ethyl (CAS NO: 188489-07-8), profluazol (CAS NO: 190314-43-3), trifludimoxazin (CAS NO: 1258836-72-4), N-ethyl-3-(2,6-dichloro-4-trifluoromethylphenoxy)-5-methyl-1H-pyrazole-1-carboxamide (CAS NO: 452098-92-9), N-tetrahydrofurfuryl-3-(2,6-dichloro-4-trifluoromethylphenoxy)-5-methyl-1H-pyrazole-1-carboxamide (CAS NO: 915396-43-9), N-ethyl-3-(2-chloro-6-fluoro-4-trifluoromethylphenoxy)-5-methyl-1H-pyrazole-1-carboxamide (CAS NO: 452099-05-7), N-tetrahydrofurfuryl-3-(2-chloro-6-fluoro-4-trifluoromethylphenoxy)-5-methyl-1H-pyrazole-1-carboxamide (CAS NO: 452100-03-7), 3-[7-Fluoro-3-oxo-4-(prop-2-ynyl)-3,4-dihydro-2H-benzo[1,4]oxazin-6-yl]-1,5-dimethyl-6-thioxo[1,3,5]triazinane-2,4-dione (CAS NO: 451484-50-7), 2-(2,2,7-trifluoro-3-oxo-4-prop-2-ynyl-3,4-dihydro-2H-benzo[1,4]oxazin-6-yl)-4,5,6,7-tetrahydroisoindole-1,3-dione (CAS NO: 1300118-96-0), Methyl (E)-4-[2-chloro-5-[4-chloro-5-(difluoromethoxy) -1H-methylpyrazol-3-yl]-4-fluorophenoxy]-3-methoxy-but-2-enoate (CAS NO: 948893-00-3), phenylpyridines disclosed in WO2016/120116, benzoxazinone derivatives disclosed in EP09163242.2, and compounds shown in general formula I

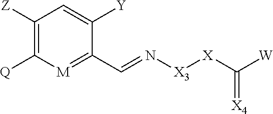

see patent CN202011462769.7);
in another exemplary embodiment, Q represents

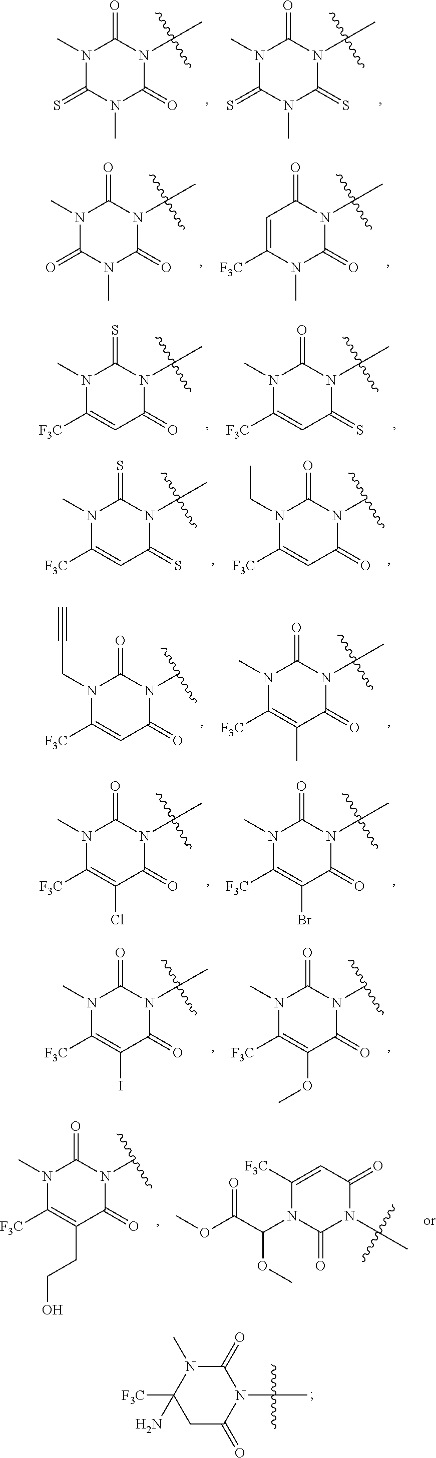

Y represents halogen, halogenated C1-C6 alkyl or cyano;
Z represents halogen;
M represents CH or N;
X represents —CX₁X₂—(C1-C6 alkyl)ₙ-, —(C1-C6 alkyl)-CX₁X₂—(C1-C6 alkyl)ₙ- or —(CH₂)r-, n represents 0 or 1, r represents an integer greater than or equal to 2;
X₁ and X₂ independently represent hydrogen, halogen, C1-C6 alkyl, C2-C6 alkenyl, C2-C6 alkynyl, halogenated C1-C6 alkyl, halogenated C2-C6 alkenyl, halogenated C2-C6 alkynyl, C3-C6 cycloalkyl, C3-C6 cycloalkyl C1-C6 alkyl, C1-C6 alkoxy, C1-C6 alkylsulphanyl, hydroxy C1-C6 alkyl, C1-C6 alkoxy C1-C6 alkyl, phenyl or benzyl;
X₃ and X₄ independently represent O or S;
W represents hydroxyl, C1-C6 alkoxy, C2-C6 alkenyloxy, C2-C6 alkynyloxy, halogenated C1-C6 alkoxy, halogenated C2-C6 alkenyloxy, halogenated C2-C6 alkynyloxy, C3-C6 cycloalkyloxy, phenoxy, sulfydryl, C1-C6 alkylsulphanyl, C2-C6 alkenylsulphanyl, C2-C6 alkynylsulphanyl, halogenated C1-C6 alkylsulphanyl, halogenated C2-C6 alkenylsulphanyl, halogenated C2-C6 alkynylsulphanyl, C3-C6 cycloalkylsulphanyl, phenylsulphanyl, amino or C1-C6 alkylamino.

In another exemplary embodiment, the compound represented by the general formula I is selected from compound A: Q represents

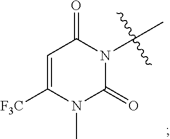

Y represents chlorine; Z represents fluorine; M represents CH; X represents —C*X₁X₂—(C1-C6 alkyl)n-(C* is chiral center, R configuration), n represents 0; X₁ represents hydrogen; X₂ represents methyl; X₃ and X₄ independently represent 0; W represents methoxy.

The PPO-inhibiting herbicides described above that are useful to carry out the present invention are often best applied in conjunction with one or more other herbicides to obtain control of a wider variety of undesirable vegetation. For example, PPO-inhibiting herbicides may further be used in conjunction with additional herbicides to which the crop plant is naturally tolerant, or to which it is resistant via expression of one or more additional transgenes as mentioned supra. When used in conjunction with other targeting herbicides, the presently claimed compounds can be formulated with the other herbicide or herbicides, tank mixed with the other herbicide or herbicides, or applied sequentially with the other herbicide or herbicides.

Suitable components for mixtures are, for example, selected from the herbicides of class b1) to b15):
b1) lipid biosynthesis inhibitors;
b2) acetolactate synthase inhibitors (ALS inhibitors);
b3) photosynthesis inhibitors;
b4) protoporphyrinogen-IX oxidase inhibitors,
b5) bleacher herbicides;
b6) enolpyruvyl shikimate 3-phosphate synthase inhibitors (EPSP inhibitors);
b7) glutamine synthetase inhibitors;
b8) 7,8-dihydropteroate synthase inhibitors (DHP inhibitors);
b9) mitosis inhibitors;
b10) inhibitors of the synthesis of very long chain fatty acids (VLCFA inhibitors);
b11) cellulose biosynthesis inhibitors;
b12) decoupler herbicides;
b13) auxinic herbicides;
b14) auxin transport inhibitors; and
b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS NO: 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters; including their agriculturally acceptable salts or derivatives.

Moreover, it may be useful to apply the PPO-inhibiting herbicides, when used in combination with other herbicidal compounds as described above, in combination with safeners. Safeners are chemical compounds which prevent or reduce damage on useful plants without having a major impact on the herbicidal action of herbicides towards unwanted plants. They can be applied either before sowings (e.g. on seed treatments, shoots or seedlings) or in the pre-emergence application or post-emergence application of the useful plant.

Furthermore, the safeners, the PPO-inhibiting herbicides and/or other herbicides can be applied simultaneously or in succession.

PPO-inhibiting herbicides, herbicidal compounds of groups b1)-b15) and safeners are known herbicides and safeners, respectively, for example, see WO2013/189984; The Compendium of Pesticide Common Names (http://www.alanwood.net/pesticides/); Farm Chemicals Handbook 2000, Volume 86, Meister Publishing Company, 2000; B. Hock, C. Fedtke, R. R. Schmidt, Herbizide [herbicide], Georg Thieme Verlag, Stuttgart, 1995; W. H. Ahrens, Herbicide Handbook, 7th edition, Weed Science Society of America, 1994, and K. K. Hatzios, Herbicide Handbook, supplement to the 7th edition, Weed Science Society of America, 1998.

The plant, to which the present invention is applied, is not particularly limited to, but includes monocotyledonous or dicotyledonous plants. Further, the plant includes herbaceous plants or woody plants. The monocotyledonous plant may include plants belonging to the family Alismataceae, Hydrocharitaceae, Juncaginaceae, Scheuchzeriaceae, Potamogetonaceae, Najadaceae, Zosteraceae, Liliaceae, Haemodoraceae, Agavaceae, Amaryllidaceae, Dioscoreaceae, Pontederiaceae, Iridaceae, Burmanniaceae, Juncaceae, Commelinaceae, Eriocaulaceae, Gramineae Poaceae, Araceae, Lemnaceae, Sparganiaceae, Typhaceae, Cyperaceae, Musaceae, Zingiberaceae, Cannaceae, Orchidaceae, but is not limited thereto.

The dicotyledonous plant may include plants belonging to the family Diapensiaceae, Clethraceae, Pyrolaceae, Ericaceae, Myrsinaceae, Primulaceae, Plumbaginaceae, Ebenaceae, Styracaceae, Symplocaceae, Oleaceae, Loganiaceae, Gentianaceae, Menyanthaceae, Apocynaceae, Asclepiadaceae, Rubiaceae, Polemoniaceae, Convolvulaceae, Boraginaceae, Verbenaceae, Labiatae, Solanaceae, Scrophulariaceae, Bignoniaceae, Acanthaceae, Pedaliaceae, Orobanchaceae, Gesneriaceae, Lentibulariaceae, Phrymaceae, Plantaginaceae, Caprifoliaceae, Adoxaceae, Valerianaceae, Dipsacaceae, Campanulaceae, Compositae, Myricaceae, Juglandaceae, Salicaceae, Betulaceae, Fagaceae, Ulmaceae, Moraceae, Urticaceae, Santalaceae, Loranthaceae, Polygonaceae, Phytolaccaceae, Nyctaginaceae, Aizoaceae, Portulacaceae, Caryophyllaceae, Chenopodiaceae, Amaranthaceae, Cactaceae, Magnoliaceae, Illiciaceae, Lauraceae, Cercidiphyllaceae, Ranunculaceae, Berberidaceae, Lardizabalaceae, Menispermaceae, Nymphaeaceae, Ceratophyllaceae, Cabombaceae, Saururaceae, Piperaceae, Chloranthaceae, Aristolochiaceae, Actinidiaceae, Theaceae, Guttiferae, Droseraceae, Papaveraceae, Capparidaceae, Cruciferae, Platanaceae, Hamamelidaceae, Crassulaceae, Saxifragaceae, Eucommiaceae, Pittosporaceae, Rosaceae, Leguminosae, Oxalidaceae, Geraniaceae, Tropaeolaceae, Zygophyllaceae, Linaceae, Euphorbiaceae, Callitrichaceae, Rutaceae, Simaroubaceae, Meliaceae, Polygalaceae, Anacardiaceae, Aceraceae, Sapindaceae, Hippocastanaceae, Sabiaceae, Balsaminaceae, Aquifoliaceae, Celastraceae, Staphyleaceae, Buxaceae, Empetraceae, Rhamnaceae, Vitaceae, Elaeocarpaceae, Tiliaceae, Malvaceae, Sterculiaceae, Thymelaeaceae, Elaeagnaceae, Flacourtiaceae, Violaceae, Passifloraceae, Tamaricaceae, Elatinaceae, Begoniaceae, Cucurbitaceae, Lythraceae, Punicaceae, Onagraceae, Haloragaceae, Alangiaceae, Cornaceae, Araliaceae, Umbelliferae (Apiaceae), but is not limited thereto.

In another exemplary embodiment, plants include but are not limited to: (1) food crops: Oryza spp., like Oryza sativa, Oryza latifolia, Oryza sativa L., Oryza glaberrima; Triticum spp., like Triticum aestivum, T. Turgidum ssp. durum; Hordeum spp., like Hordeum vulgare, Hordeum arizonicum; Secale cereale; Avena spp., like Avena sativa, Avena fatua, Avena byzantine, Avena fatua var. sativa, Avena hybrida; Echinochloa spp., like Pennisetum glaucum, Sorghum (Sorghum bicolor), Sorghum vulgare, Triticale, Zea mays or corn, Millet, Rice, Foxtail millet, Proso millet, Sorghum bicolor, Panicum, Fagopyrum spp., Panicum miliaceum, Setaria italica, Zizania palustris, Eragrostis tef, Panicum miliaceum, Eleusine coracana; (2) legume crops: Glycine spp. like Glycine max, Vicia spp., Vigna spp., Pisum spp., field bean, Lupinus spp., Vicia, Tamarindus indica, Lens culinaris, Lathyrus spp., Lablab, broad bean, mung bean, red bean, chickpea; (3) oil crops: Arachis hypogaea, Arachis spp, Sesamum spp., Helianthus spp. like Helianthus annuus, Elaeis like Eiaeis guineensis and Elaeis oleifera, rape, Brassica napus, Sesamum orientale, Brassica juncea, Oilseed rape, Camellia oleifera, oil palm, olive, castor-oil plant, Brassica napus L., canola; (4) fiber crops: Agave sisalana, Gossypium spp. like Gossypium and Gossypium barbadense, Gossypium hirsutum, Hibiscus cannabinus, Agave sisalana, Musa textilis Nee, Linum usitatissimum, Corchorus capsularis L, Boehmeria nivea (L.), Cannabis sativa, Cannabis sativa; (5) fruit crops: Ziziphus spp., Cucumis spp., Passiflora edulis, Vitis spp., Vaccinium spp., Pyrus communis, Prunus spp., Psidium spp., Punica granatum, Malus spp., Citrullus lanatus, Citrus spp., Ficus carica, Fortunella spp., Fragaria spp. (strawberry), Crataegus spp., Diospyros spp., Eugenia unifora, Eriobotrya japonica, Dimocarpus longan, Carica papaya, Cocos spp., Averrhoa carambola, Actinidia spp., Prunus amygdalus, Musa spp. (banana), Persea spp. (Persea Americana), Psidium guajava, Mammea Americana, Mangifera indica, Canarium album (Olea europaea), Cocos nucifera, Malpighia emarginata, Manilkara zapota, Ananas comosus, Annona spp., Citrus reticulate (Citrus spp.), Artocarpus spp., Litchi chinensis, Ribes spp., Rubus spp., pear, peach, apricot, plum, red bayberry, lemon, kumquat, durian, orange, blueberry, hami melon, muskmelon, date palm, walnut tree, cherry tree; (6) rhizome crops: Manihot spp., Ipomoea batatas, Colocasia esculenta, tuber mustard, Allium cepa (onion), eleocharis tuberose (water chestnut), Cyperus rotundus, Rhizoma dioscoreae; (7) vegetable crops: Spinacia spp., Phaseolus spp., Lactuca sativa, Momordica spp, Petroselinum crispum, Capsicum spp., Solanum spp. (such as Solanum tuberosum, Solanum integrifolium, Solanum lycopersicum), Lycopersicon spp. (such as Lycopersicon esculentum, Lycopersicon lycopersicum, Lycopersicon pyriforme), Macrotyloma spp., Kale, Luffa acutangula, lentil, okra, onion, potato, artichoke, asparagus, broccoli, Brussels sprouts, cabbage, carrot, cauliflower, celery, collard greens, squash, Benincasa hispida, Asparagus officinalis, Apium graveolens, Amaranthus spp., Allium spp., Abelmoschus spp., Cichorium endivia, Cucurbita spp., Coriandrum sativum, B. carinata, Rapbanus sativus, Brassica spp. (such as Brassica rapa ssp., canola, turnip rape, leaf mustard, cabbage, black mustard, Brussels sprout, Solanaceae (eggplant), sweet pepper, cucumber, luffa, Chinese cabbage, rape, calabash, Chinese chives, lotus, lotus root, lettuce; (8) flower crops: Tropaeolum minus, Tropaeolum majus, Canna indica, Opuntia spp., Tagetes spp., Cymbidium (orchid), Crinum asiaticum L., Clivia, Hippeastrum rutilum, Rosa rugosa, Rosa Chinensis, Jasminum sambac, Tulipa gesneriana L., Cerasus sp. Pharbitis nil (L.) Choisy, Calendula officinalis L., Nelumbo sp., Bellis perennis L., Dianthus caryophyllus, Petunia hybrida, Tulipa gesneriana L., Lilium brownii, Prunus mume, Narcissus tazetta L., Jasminum nudiflorum Lindl., Primula malacoides, Daphne odora, Camellia japonica, Michelia alba, Magnolia liliiflora, Viburnum macrocephalum, Clivia miniata, Malus spectabilis, Paeonia suffruticosa, Paeonia lactiflora, Syzygium aromaticum, Rhododendron simsii, Rhododendron hybridum, Michelia figo (Lour.) Spreng., Cercis chinensis, Kerria japonica, Weigela florida, Fructus forsythiae, Jasminum mesnyi, Parochetus communis, Cyclamen persicum Mill., Phalaenophsis hybrid, Dendrobium nobile, Hyacinthus orientalis, Iris tectorum Maxim, Zantedeschia aethiopica, Calendula officinalis, Hippeastrum rutilum, Begonia semperflorens hybr, Fuchsia hybrida, Begonia maculate Raddi, Geranium; (9) medicinal crops: Carthamus tinctorius, Mentha spp., Rheum rhabarbarum, Crocus sativus, Lycium chinense, Polygonatum odoratum, Polygonatum Kingianum, Anemarrhena asphodeloides Bunge, Radix ophiopogonis, Fritillaria cirrhosa, Curcuma aromatica, Amomum villosum Lour., Polygonum multiflorum, Rheum officinale, Glycyrrhiza uralensis Fisch, Astragalus membranaceus, Panax ginseng, Panax notoginseng, Acanthopanax gracilistylus, Angelica sinensis, Ligusticum wallichii, Bupleurum sinenses DC., Datura stramonium Linn., Datura metel L., Mentha haplocalyx, Leonurus sibiricus L., Agastache rugosus, Scutellaria baicalensis, Prunella vulgaris L., Pyrethrum carneum, Cinchona ledgeriana, Hevea brasiliensis (wild), Piper Nigrum L.; (10) raw material crops: Hevea brasiliensis, Ricinus communis, Vernicia fordii, Morus alba L., Hops Humulus lupulus, Betula, Alnus cremastogyne Burk., Rhus verniciflua stokes; (11) pasture crops: Agropyron spp., Trifolium spp., Miscanthus sinensis, Pennisetum sp., Phalaris arundinacea, Panicum virgatum, prairie grasses, Indiangrass, Big bluestem grass, Phleum pratense, turf, cyperaceae (Kobresia pygmaea, Carex pediformis, Carex humilis), Medicago sativa Linn, Phleum pratense L., Medicago sativa, Melilotus suavcolen, Astragalus sinicus, Crotalaria juncea, Sesbania cannabina, Azolla imbircata, Eichhornia crassipes, Amorpha fruticosa, Lupinus micranthus, Trifolium, Astragalus adsurgens pall, Pistia stratiotes linn, Alternanthera philoxeroides, Lolium; (12) sugar crops: Saccharum officinarum (Saccharum spp.), Beta vulgaris; (13) beverage crops: Camellia sinensis, Camellia Sinensis, tea, Coffee (Coffea spp.), Theobroma cacao, Humulus lupulus Linn.; (14) lawn plants: Ammophila arenaria, Poa spp. (Poa pratensis (bluegrass)), Agrostis spp. (Agrostis matsumurae, Agrostis palustris), Lolium spp. (Lolium), Festuca spp. (Festuca ovina L.), Zoysia spp. (Zoysia japonica), Cynodon spp. (Cynodon dactylon/Bermuda grass), Stenotaphrum secunda turn (Stenotaphrum secunda turn), Paspalum spp. (Paspalum notatum), Eremochloa ophiuroides (centipede grass), Axonopus spp. (carpetweed), Bouteloua dactyloides (buffalo grass), Bouteloua var. spp. (Bouteloua gracilis), Digitaria sanguinalis, Cyperus rotundus, Kyllinga brevifolia, Cyperus amuricus, Erigeron canadensis, Hydrocotyle sibthorpioides, Kummerowia striata, Euphorbia humifusa, Viola arvensis, Carex rigescens, Carex heterostachya, turf; (15) tree crops: Pinus spp., Salix spp., Acer spp., Hibiscus spp., Eucalyptus spp., Ginkgo biloba, Bambusa sp., Populus spp., Prosopis spp., Quercus spp., Phoenix spp., Fagus spp., Ceiba pentandra, Cinnamomum spp., Corchorus spp., Phragmites australis, Physalis spp., Desmodium spp., Populus, Hedera helix, Populus tomentosa Carr, Viburnum odoratissinum, Ginkgo biloba L., Quercus, Ailanthus altissima, Schima superba, Ilex purpurea, Platanus acerifolia, ligustrum lucidum, Buxus megistophylla Levl., Dahurian larch, Acacia mearnsii, Pinus massoniana, Pinus khasys, Pinus yunnanensis, Pinus finlaysoniana, Pinus tabuliformis, Pinus koraiensis, Juglans nigra, Citrus limon, Platanus acerifolia, Syzygium jambos, Davidia involucrate, Bombax malabarica L., Ceiba pentandra (L.), Bauhinia blakeana, Albizia saman, Albizzia julibrissin, Erythrina corallodendron, Erythrina indica, Magnolia gradiflora, Cycas revolute, Lagerstroemia indica, coniferous, macrophanerophytes, Frutex, Morus alba L.; (16) nut crops: Bertholletia excelsea, Castanea spp., Corylus spp., Carya spp., Juglans spp., Pistacia vera, Anacardium occidentale, Macadamia (Macadamia integrifolia), Carya illinoensis Koch, Macadamia, Pistachio, Badam, other plants that produce nuts; (17) others: Arabidopsis thaliana, Brachiaria eruciformis, Cenchrus echinatus, Setaria faberi, Eleusine indica, Cadaba farinose, algae, Carex elata, ornamental plants, Carissa macrocarpa, Cynara spp., Daucus carota, Dioscorea spp., Erianthus sp., Festuca arundinacea, Hemerocallis fulva, Lotus spp., Luzula sylvatica, Medicago sativa, Melilotus spp., Morus nigra, Nicotiana spp., Olea spp., Ornithopus spp., Pastinaca sativa, Sambucus spp., Sinapis sp., Syzygium spp., Tripsacum dactyloides, Triticosecale rimpaui, Viola odorata, and the like.

In one exemplary embodiment, the plant is rice (Oryza sativa L.), sorghum (Sorghum bicolor), wheat (Triticum aestivum), barley (Hordeum vulgare), foxtail millet (Setaria italica), corn (Zea mays), sugarcane (Saccharum officinarum), Arabidopsis thaliana, soybean (Glycine max), peanut (Arachis hypogaea), tobacco (Nicotiana tabacum), cotton (Gossypium hirsutum), radish (Raphanus sativus), cabbage (Brassica oleracea), sweet potato (Dioscorea esculenta), yam (Dioscorea cayenensis), cassava (Manihot esculenta), potato (Solanum tuberosum), tomato (Solanum lycopersicum), pepper (Capsicum annuum), eggplant (Solanum melongena), watermelon (Citrullus lanatus), squash (Cucurbita moschata), cucumber (Cucumis sativus), lettuce (Lactuca sativa), sesame (Sesamum indicum), oilseed rape (Brassica napus), sunflower (Helianthus annuus), mulberry (Morus alba), cowpea (Vigna unguiculata), strawberry (Fragaria ananassa), apple (Malus domestica), peach (Prunus persica), cherry (Prunus pseudocerasus), apricot (Prunus armeniaca), grape vine (Vitis vinfera), papaya (Carica papaya) or alfalfa (Medicago sativa).

In the present invention, the term “plant tissue” or “plant part” includes plant cells, protoplasts, plant tissue cultures, plant callus, plant blocks, and plant embryos, pollens, ovules, seeds, leaves, stems, flowers, branches, seedlings, fruits, cores, spikes, roots, root tips, anthers, etc.

In the present invention, the “plant cell” should be understood to mean any cell derived from or found in a plant that is capable of forming, for example, an undifferentiated tissue such as callus, a differentiated tissue such as an embryo, a plant part, a plant or a seed.

In the present invention, the “host organism” should be understood to mean any single or multicellular organism into which a nucleic acid encoding a mutant protein can be introduced, including, for example, bacteria such as Eescherichia coli, fungi such as yeast (e.g., Saccharomyces cerevisiae), molds (e.g., aspergillus), plant cells, plants and the like.

In one aspect, the present invention discloses a PPO polypeptide or a bioactive fragment thereof tolerant to a PPO-inhibiting herbicide, and the polypeptide comprises the motif “LLLNYI” (namely “leucine-leucine-leucine-aspartyl-tyrosine-isoleucine”) wherein the leucine L at position 3 within the motif is substituted with any other amino acid, or the tyrosine Y at position 5 is substituted with any other amino acid.

In one embodiment, within the motif “LLLNYI”, the leucine L at position 3 is mutated to serine S, abbreviated as “LLSNYI”; or
the leucine L at position 3 is mutated to isoleucine I, abbreviated as “LLINYI”; or
the leucine L at position 3 is mutated to glycine G, abbreviated as “LLGNYI”; or
the leucine L at position 3 is mutated to threonine T, abbreviated as “LLTNYI”; or
the leucine L at position 3 is mutated to valine V, abbreviated as “LLVNYI”; or
the leucine L at position 3 is mutated to tryptophan W, abbreviated as “LLWNYI”; or
the tyrosine Y at position 5 is mutated to methionine M, abbreviated as “LLLNMI”; or
the tyrosine Y at position 5 is mutated to isoleucine I, abbreviated as “LLLNII”; or
the tyrosine Y at position 5 is mutated to leucine L, abbreviated as “LLLNLI”; or
the tyrosine Y at position 5 is mutated to valine V, abbreviated as “LLLNVI”.

In another embodiment, within the motif “LLLNYI”, the leucine L at position 3 is substituted with any other amino acid and the tyrosine Y at position 5 is substituted with any other amino acid.

In another embodiment, within the motif “LLLNYI”, the leucine L at position 3 is mutated to serine S and the tyrosine Y at position 5 is mutated to isoleucine I, abbreviated as “LLSNII”; or
the leucine L at position 3 is mutated to threonine T and the tyrosine Y at position 5 is mutated to isoleucine I, abbreviated as “LLTNII”; or
the leucine L at position 3 is mutated to threonine T and the tyrosine Y at position 5 is mutated to valine V, abbreviated as “LLTNVI”; or
the leucine L at position 3 is mutated to serine S and the tyrosine Y at position 5 is mutated to valine V, abbreviated as “LLSNVI”; or
the leucine L at position 3 is mutated to valine V and the tyrosine Y at position 5 is mutated to leucine L, abbreviated as “LLVNLI”; or
the leucine L at position 3 is mutated to tryptophan W and the tyrosine Y at position 5 is mutated to leucine L, abbreviated as “LLWNLI”.

In one embodiment, the polypeptide comprises the mutant of freely-combined amino acid sequence and a fragment thereof that has at least 60%, at least 65%, at least 70%, at least 75%, at least 80%, at least 85%, at least 90%, at least 95%, at least 96%, at least 97%, at least 98% or at least 99% sequence identity to the amino acid sequence as set forth in any one from SEQ ID NO: 1-19, and the mutant comprises one or more amino acid mutations as defined above.

In another embodiment, the polypeptide has amino acid sequence as set forth in any one from SEQ ID NO: 1-19, except that it has one or more amino acid mutations as defined above; preferably, the amino acid sequence of the polypeptide is as set forth in any one from SEQ ID NO: 1-19, except for one or more amino acid mutations as defined above.

In another embodiment, as compared to the amino acid sequence of a wild-type rice PPO1, the amino acid sequence of the PPO polypeptide has one or more mutations at one or more positions (only) corresponding to 423 and 425 of the amino acid sequence of wild-type rice PPO1 protein as set forth in SEQ ID NO: 1; or
as compared to the amino acid sequence of a wild-type corn PPO1, the amino acid sequence of the PPO polypeptide has one or more mutations at one or more positions (only) corresponding to 424 and 426 of the amino acid sequence of wild-type corn PPO1 protein as set forth in SEQ ID NO: 2; or
as compared to the amino acid sequence of a wild-type oilseed rape PPO1, the amino acid sequence of the PPO polypeptide has one or more mutations at one or more positions (only) corresponding to 424 and 426 of the amino acid sequence of wild-type oilseed rape PPO1 protein as set forth in SEQ ID NO: 3; or
as compared to the amino acid sequence of a wild-type oilseed rape PPO1, the amino acid sequence of the PPO polypeptide has one or more mutations at one or more positions (only) corresponding to 423 and 425 of the amino acid sequence of wild-type oilseed rape PPO1 protein as set forth in SEQ ID NO: 4; or
as compared to the amino acid sequence of a wild-type peanut PPO1, the amino acid sequence of the PPO polypeptide has one or more mutations at one or more positions (only) corresponding to 445 and 447 of the amino acid sequence of wild-type peanut PPO1 protein as set forth in SEQ ID NO: 5; or
as compared to the amino acid sequence of a wild-type peanut PPO1, the amino acid sequence of the PPO polypeptide has one or more mutations at one or more positions (only) corresponding to 439 and 441 of the amino acid sequence of wild-type peanut PPO1 protein as set forth in SEQ ID NO: 6; or
as compared to the amino acid sequence of a wild-type soybean PPO1, the amino acid sequence of the PPO polypeptide has one or more mutations at one or more positions (only) corresponding to 430 and 432 of the amino acid sequence of wild-type soybean PPO1 protein as set forth in SEQ ID NO: 7; or
as compared to the amino acid sequence of a wild-type sorghum PPO1, the amino acid sequence of the PPO polypeptide has one or more mutations at one or more positions (only) corresponding to 423 and 425 of the amino acid sequence of wild-type sorghum PPO1 protein as set forth in SEQ ID NO: 8; or
as compared to the amino acid sequence of a wild-type wheat PPO1, the amino acid sequence of the PPO polypeptide has one or more mutations at one or more positions (only) corresponding to 418 and 420 of the amino acid sequence of wild-type wheat PPO1 protein as set forth in SEQ ID NO: 9, 10 or 11; or
as compared to the amino acid sequence of a wild-type tomato PPO1, the amino acid sequence of the PPO polypeptide has one or more mutations at one or more positions (only) corresponding to 445 and 447 of the amino acid sequence of wild-type tomato PPO1 protein as set forth in SEQ ID NO: 12; or
as compared to the amino acid sequence of a wild-type potato PPO1, the amino acid sequence of the PPO polypeptide has one or more mutations at one or more positions (only) corresponding to 444 and 446 of the amino acid sequence of wild-type potato PPO1 protein as set forth in SEQ ID NO: 13; or
as compared to the amino acid sequence of a wild-type tobacco PPO1, the amino acid sequence of the PPO polypeptide has one or more mutations at one or more positions (only) corresponding to 440 and 442 of the amino acid sequence of wild-type tobacco PPO1 protein as set forth in SEQ ID NO: 14; or
as compared to the amino acid sequence of a wild-type Arabidopsis thaliana PPO1, the amino acid sequence of the PPO polypeptide has one or more mutations at one or more positions (only) corresponding to 423 and 425 of the amino acid sequence of wild-type Arabidopsis thaliana PPO1 protein as set forth in SEQ ID NO: 15; or
as compared to the amino acid sequence of a wild-type upland cotton PPO1, the amino acid sequence of the PPO polypeptide has one or more mutations at one or more positions (only) corresponding to 426 and 428 of the amino acid sequence of wild-type upland cotton PPO1 protein as set forth in SEQ ID NO: 16; or
as compared to the amino acid sequence of a wild-type radish PPO1, the amino acid sequence of the PPO polypeptide has one or more mutations at one or more positions (only) corresponding to 425 and 427 of the amino acid sequence of wild-type radish PPO1 protein as set forth in SEQ ID NO: 17; or
as compared to the amino acid sequence of a wild-type foxtail millet PPO1, the amino acid sequence of the PPO polypeptide has one or more mutations at one or more positions (only) corresponding to 422 and 424 of the amino acid sequence of wild-type foxtail millet PPO1 protein as set forth in SEQ ID NO: 18; or
as compared to the amino acid sequence of a wild-type cabbage PPO1, the amino acid sequence of the PPO polypeptide has one or more mutations at one or more positions (only) corresponding to 424 and 426 of the amino acid sequence of wild-type cabbage PPO1 protein as set forth in SEQ ID NO: 19.

In another embodiment, as compared to the amino acid sequence of a wild-type rice PPO1, the amino acid sequence of the PPO polypeptide has one or more mutations selected from the group consisting of L423S, L423I, L423G, Y425M, Y425I and Y425V at one or more positions (only) corresponding to 423 and 425 of the amino acid sequence of wild-type rice PPO1 protein as set forth in SEQ ID NO: 1; preferably, it has the following mutations: L423S/Y425I; or
as compared to the amino acid sequence of a wild-type corn PPO1, the amino acid sequence of the PPO polypeptide has one or more mutations selected from the group consisting of L424T, L424S, L424V, Y424W, Y426V, Y426I and Y426 L at one or more positions (only) corresponding to 424 and 426 of the amino acid sequence of wild-type corn PPO1 protein as set forth in SEQ ID NO: 2; preferably, it has the following mutations: L424T/Y426V, L424S/Y426V, L424V/Y426L, L424W/Y426 L or L424S/Y426I; or
as compared to the amino acid sequence of a wild-type oilseed rape PPO1, the amino acid sequence of the PPO polypeptide has one or more mutations selected from the group consisting of L424S and Y426I at one or more positions (only) corresponding to 424 and 426 of the amino acid sequence of wild-type oilseed rape PPO1 protein as set forth in SEQ ID NO: 3; preferably, it has the following mutations: L424S/Y426I; or
as compared to the amino acid sequence of a wild-type oilseed rape PPO1, the amino acid sequence of the PPO polypeptide has one or more mutations selected from the group consisting of L423S and Y425I at one or more positions (only) corresponding to 423 and 425 of the amino acid sequence of wild-type oilseed rape PPO1 protein as set forth in SEQ ID NO: 4; preferably, it has the following mutations: L423S/Y425I; or
as compared to the amino acid sequence of a wild-type peanut PPO1, the amino acid sequence of the PPO polypeptide has one or more mutations selected from the group consisting of L445S and Y447I at one or more positions (only) corresponding to 445 and 447 of the amino acid sequence of wild-type peanut PPO1 protein as set forth in SEQ ID NO: 5; preferably, it has the following mutations: L445S/Y447I; or
as compared to the amino acid sequence of a wild-type peanut PPO1, the amino acid sequence of the PPO polypeptide has one or more mutations selected from the group consisting of L439S and Y441I at one or more positions (only) corresponding to 439 and 441 of the amino acid sequence of wild-type peanut PPO1 protein as set forth in SEQ ID NO: 6; preferably, it has the following mutations: L439S/Y441I; or
as compared to the amino acid sequence of a wild-type soybean PPO1, the amino acid sequence of the PPO polypeptide has one or more mutations selected from the group consisting of L430S and Y432I at one or more positions (only) corresponding to 430 and 432 of the amino acid sequence of wild-type soybean PPO1 protein as set forth in SEQ ID NO: 7; preferably, it has the following mutations: L430S/Y432I; or
as compared to the amino acid sequence of a wild-type sorghum PPO1, the amino acid sequence of the PPO polypeptide has one or more mutations selected from the group consisting of L423S and Y425I at one or more positions (only) corresponding to 423 and 425 of the amino acid sequence of wild-type sorghum PPO1 protein as set forth in SEQ ID NO: 8; preferably, it has the following mutations: L423S/Y425I; or
as compared to the amino acid sequence of a wild-type wheat PPO1, the amino acid sequence of the PPO polypeptide has one or more mutations selected from the group consisting of L418S and Y420I at one or more positions (only) corresponding to 418 and 420 of the amino acid sequence of wild-type wheat PPO1 protein as set forth in SEQ ID NO: 9, 10 or 11; preferably, it has the following mutations: L418S/Y420I; or
   as compared to the amino acid sequence of a wild-type tomato PPO1, the amino acid sequence of the PPO polypeptide has one or more mutations selected from the group consisting of L445S and Y447I at one or more positions (only) corresponding to 445 and 447 of the amino acid sequence of wild-type tomato PPO1 protein as set forth in SEQ ID NO: 12; preferably, it has the following mutations: L445S/Y447I; or
as compared to the amino acid sequence of a wild-type potato PPO1, the amino acid sequence of the PPO polypeptide has one or more mutations selected from the group consisting of L444S and Y446I at one or more positions (only) corresponding to 444 and 446 of the amino acid sequence of wild-type potato PPO1 protein as set forth in SEQ ID NO: 13; preferably, it has the following mutations: L444S/Y446I; or
as compared to the amino acid sequence of a wild-type tobacco PPO1, the amino acid sequence of the PPO polypeptide has one or more mutations selected from the group consisting of L440S and Y442I at one or more positions (only) corresponding to 440 and 442 of the amino acid sequence of wild-type tobacco PPO1 protein as set forth in SEQ ID NO: 14; preferably, it has the following mutations: L440S/Y442I; or
as compared to the amino acid sequence of a wild-type Arabidopsis thaliana PPO1, the amino acid sequence of the PPO polypeptide has one or more mutations selected from the group consisting of L423S and Y425I at one or more positions (only) corresponding to 423 and 425 of the amino acid sequence of wild-type Arabidopsis thaliana PPO1 protein as set forth in SEQ ID NO: 15; preferably, it has the following mutations: L423S/Y425I; or
as compared to the amino acid sequence of a wild-type upland cotton PPO1, the amino acid sequence of the PPO polypeptide has one or more mutations selected from the group consisting of L426S and Y428I at one or more positions (only) corresponding to 426 and 428 of the amino acid sequence of wild-type upland cotton PPO1 protein as set forth in SEQ ID NO: 16; preferably, it has the following mutations: L426S/Y428I; or
as compared to the amino acid sequence of a wild-type radish PPO1, the amino acid sequence of the PPO polypeptide has one or more mutations selected from the group consisting of L425S and Y427I at one or more positions (only) corresponding to 425 and 427 of the amino acid sequence of wild-type radish PPO1 protein as set forth in SEQ ID NO: 17; preferably, it has the following mutations: L425S/Y427I; or
as compared to the amino acid sequence of a wild-type foxtail millet PPO1, the amino acid sequence of the PPO polypeptide has one or more mutations selected from the group consisting of L422S and Y424I at one or more positions (only) corresponding to 422 and 424 of the amino acid sequence of wild-type foxtail millet PPO1 protein as set forth in SEQ ID NO: 18; preferably, it has the following mutations: L422S/Y424I; or
as compared to the amino acid sequence of a wild-type cabbage PPO1, the amino acid sequence of the PPO polypeptide has one or more mutations selected from the group consisting of L424S and Y426I at one or more positions (only) corresponding to 424 and 426 of the amino acid sequence of wild-type cabbage PPO1 protein as set forth in SEQ ID NO: 19; preferably, it has the following mutations: L424S/Y426I.

In another embodiment, the polypeptide has an amino acid sequence as set forth in any one from SEQ ID NO: 20-48; preferably, the amino acid sequence of the polypeptide is as set forth in any one from SEQ ID NO: 20-48.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

The terms “protein”, “polypeptide” and “peptide” can be used interchangeably in the present invention and refer to a polymer of amino acid residues, including polymers of chemical analogs in which one or more amino acid residues are natural amino acid residues. The proteins and polypeptides of the present invention may be recombinantly produced or chemically synthesized.

For the terms regarding amino acid substitutions used in the specification, the first letter represents a naturally occurring amino acid at a certain position in a particular sequence, the following number represents the position corresponding to the SEQ ID NO: 1, and the second letter represents a different amino acid substituting for the naturally occurring amino acid. For example, L423S represents that the leucine at position 423 is substituted with serine relative to the amino acid sequence of SEQ ID NO: 1. For double or multiple mutations, each mutation is separated by “/”. For example, L423S/Y425I means that, relative to the amino acid sequence of SEQ ID NO: 1, the leucine at position 423 is substituted with serine, and the tyrosine at position 425 is substituted with isoleucine, and both mutations are present in the specific mutant OsPPO1 protein.

A particular amino acid position (numbering) within the protein of the present invention is determined by aligning the amino acid sequence of a protein of interest with SEQ ID NO: 1 or SEQ ID NO: 2-19, etc. using a standard sequence alignment tool, for example, Smith-Waterman algorithm or CLUSTALW2 algorithm is used to align two sequences, wherein the sequences are considered to be aligned when the alignment score is the highest. The alignment score can be calculated according to the method described in Wilbur, W. J. and Lipman, D. J. (1983), “Rapid similarity searches of nucleic acid and protein data banks”, Proc. Natl. Acad. Sci. USA, 80: 726-730. The default parameters used in the ClustalW2 (1.82) algorithm are preferably: protein gap opening penalty=10.0; protein gap extension penalty=0.2; protein matrix=Gonnet; protein/DNA end gap=−1; and protein/DNA GAPDIST=4.

Preferably, the AlignX program (a part of the vector NTI set) is used to match the default parameters for the multiple alignment (gap opening penalty: 10 og, gap extension penalty: 0.05), and the position of a particular amino acid within a protein of the present invention is determined by aligning the amino acid sequence of the protein with SEQ ID NO: 1.

The identity of amino acid sequences can be determined by conventional methods using the BLAST algorithm (Altschul et al., 1990, Mol. Biol. 215:403-10) available from the National Center for Biotechnology Information (www.ncbi.nlm.nih.gov/) with the default parameters.

It will also be apparent for a person skilled in the art that the structure of a protein can be altered, without adversely affecting its activity and functionality, for example, one or more conservative amino acid substitutions can be introduced into the amino acid sequence of the protein without adversely affecting the activity and/or three-dimensional configuration of the protein molecule. A person skilled in the art knows examples and embodiments of conservative amino acid substitutions. Specifically, an amino acid residue at certain site may be substituted with another amino acid residue belonging to the same group as the amino acid to be substituted, that is, a non-polar amino acid residue is substituted with another non-polar amino acid residue, a polar uncharged amino acid residue is substituted with another polar uncharged amino acid residue, a basic amino acid residue is substituted with another basic amino acid residue, and an acidic amino acid residue is substituted with an acidic amino acid residue. As long as a substitution does not impair the biological activity of the protein, such a conservative substitution that one amino acid is substituted by other amino acid which belong to the same group falls within the scope of the present invention.

Accordingly, the mutant protein of the present invention may further contain one or more other mutations such as conservative substitutions in the amino acid sequence in addition to the above mutations. In addition, the invention also encompasses mutant proteins that further contain one or more other non-conservative substitutions, so long as the non-conservative substitutions do not significantly affect the desired function and biological activity of the protein of the present invention.

As is well known in the art, one or more amino acid residues can be deleted from the N- and/or C-terminus of a protein, and the protein still retains the function and activity. Accordingly, in another aspect, the present invention also relates to fragments which lack one or more amino acid residues at the N- and/or C-terminus of a mutant protein while retaining the desired function and activity. Within the scope of the invention, and the fragments are referred to as bioactive fragments. In the present invention, the “bioactive fragment” refers to a portion of a mutant protein of the present invention which retains the biological activity of the mutant protein of the present invention. For example, a bioactive fragment of a mutant protein may be a bioactive fragment that lacks a moiety of one or more (for example, 1-50, 1-25, 1-10 or 1-5, e.g., 1, 2, 3, 4 or 5) of amino acid residues at the N- and/or C-terminus of the protein, but still retains the desired biological activity of the full-length protein.

As used herein, the term “mutation” refers to a single amino acid variation in a polypeptide and/or at least a single nucleotide variation in a nucleic acid sequence relative to the normal sequence or wild-type sequence or a reference sequence. In some embodiments a mutation refers to a single amino acid variation in a polypeptide and/or at least a single nucleotide variation in a nucleic acid sequence relative to a nucleotide or amino acid sequence of a PPO protein that is not herbicide resistant. In certain embodiments, mutation refers to having one or more mutations at the amino acid location corresponding to the reference PPO amino acid sequence, for example, as set forth in any one from SEQ ID NO: 1-19 or at the homologous location of a homologous gene from a different species. In certain embodiments, a mutation may include a substitution, a deletion, an inversion or an insertion. In some embodiments, a substitution, deletion, insertion, or inversion may include a variation at 1, 2, 3, 4, 5, 6, 7, 8, 9, 10, 11, 12, 13, 14, 15, 16, 17, 18, 19, 20, 21, 22, 23 or 24 nucleotides. In some embodiments, a substitution, deletion, insertion, or inversion may include a variation at 1, 2, 3, 4, 5, 6, 7 or 8 amino acid positions.

The term “wild type” is relative to mutation, which refers to the phenotype with the highest frequency in a specific population, or a system, organism and gene that has this phenotype. In some instances, a wild-type allele refers to the standard allele at a locus, or the allele having the highest frequency in a particular population, and may be represented by a particular amino acid or nucleic acid sequence. For example, a wild-type rice PPO protein may be represented by SEQ ID NO: 1. For example, a wild-type corn PPO protein may be represented by SEQ ID NO: 2.

In another aspect, the present invention also provides an isolated polynucleotide comprising a nucleic acid sequence selected from the group consisting of:
(1) a nucleic acid sequence encoding the PPO polypeptide or a biologically active fragment thereof, or a partial sequence thereof, or a complementary sequence thereof,
(2) a nucleic acid sequence that hybridizes to the sequence shown in (1) under stringent conditions; and
(3) a nucleic acid sequence encoding the same amino acid sequence as the sequence shown in (1) due to degeneracy of genetic code, or a complementary sequence thereof.

In one embodiment, the polynucleotide is a DNA molecule.

The terms “polynucleotide”, “nucleic acid”, “nucleic acid molecule” or “nucleic acid sequence” are used interchangeably to refer to an oligonucleotide, nucleotide or polynucleotide, and fragments or portions thereof, which may be single or double stranded, and represent the sense or antisense strand. A nucleic acid may include DNA, RNA or hybrids thereof, and may be of natural or synthetic origin. For example, a nucleic acid may include mRNA or cDNA. Nucleic acid may include nucleic acid that has been amplified {e.g., using polymerase chain reaction}. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymine (uracil if RNA); “M” means adenine or cytosine; “K” means guanine or thymine; and “W” means adenine or thymine. The term “isolated”, when referring to a nucleic acid refers to a nucleic acid that is apart from a substantial portion of the genome in which it naturally occurs and/or is substantially separated from other cellular components which naturally accompany such nucleic acid. For example, any nucleic acid that has been produced synthetically (e.g., by serial base condensation) is considered to be isolated. Likewise, nucleic acids that are recombinantly expressed, cloned, produced by a primer extension reaction (e.g., PCR), or otherwise excised from a genome are also considered to be isolated.

It will be apparent for a person skilled in the art that a variety of different nucleic acid sequences can encode the amino acid sequences disclosed herein due to the degeneracy of genetic codes. A person skilled in the art is able to generate additional nucleic acid sequences encoding a same protein, and thus the present invention encompasses nucleic acid sequences encoding the same amino acid sequence due to the degeneracy of genetic codes. For example, in order to achieve high expression of a heterologous gene in a host organism, such as a plant, the gene can be optimized using host-preferred codons for better expression.

The present invention also provides a plant genome comprising the polynucleotide.

In one embodiment, the plant genome is modified with at least one mutation. In another embodiment, the plant genome is modified with at least two mutations.

In one embodiment, the plastid PPO gene is modified by the plant genome mutation, such as rice plastid OsPPO1. In another embodiment, the plastid PPO gene allele is modified by the plant genome mutation, such as BnPPO1-C5 or BnPPO1-A10.

The present invention also provides a vector construct comprising the polynucleotide and the homologous or non-homologous promoter operably linked thereto.

The present invention also provides a host cell comprising the polynucleotide or the vector construct.

In one embodiment, the host cell is a plant cell.

The present invention also provides a producing method of a plant cell to gain or improve its tolerance to a PPO-inhibiting herbicide, comprising producing the above polynucleotide or the above vector construct in the plant cell by using gene editing method, or introducing the above polynucleotide or the above vector construct into the plant cell by using transgenic method.

The present invention also provides a producing method of a plant to gain or improve its tolerance to a PPO-inhibiting herbicide, comprising regenerating the above plant cell or a plant cell produced by the above method.

The present invention also provides a plant produced by the above method.

In one embodiment, the above plant or plant cell is non-transgenic.

In another embodiment, the above plant or plant cell is transgenic.

The term “transgenic” plant refers to a plant that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

The terms “gene-edited plant”, “gene-edited plant part” or “gene-edited plant cell” refer to a plant, plant part or plant cell thereof comprising one or more endogenous genes edited through a gene-editing system. The term “gene editing system” refers to a protein, nucleic acid, or combination thereof that is capable of modifying a target locus of an endogenous DNA sequence when introduced into a cell. Numerous gene editing systems suitable for use in the methods of the present invention are known in the art including, but not limited to, zinc-finger nuclease systems (ZFNs), transcription activator-like effector nuclease systems (TALEN), and CRISPR/Cas systems. The term “gene editing” as used in the present invention usually refers to a technique by which DNA is inserted, deleted, modified or replaced in a genome. For example, the gene editing may include a knock-in method which may be a common operating method used by a person skilled in the art. See “Gene Target: A Practical Method” (Edited by Joyner, Oxford University Press, 2000) by reference.

The present invention also provides a method of enabling a plant to gain or improve tolerance to a PPO-inhibiting herbicide, comprising introducing a modification in the gene encoding a protein with PPO activity to produce the PPO polypeptide or a bioactive fragment thereof.

The present invention also provides a method of gaining or improving the tolerance of a plant cell, plant tissue, plant part or plant to a PPO-inhibiting herbicide, comprising expressing the PPO polypeptide or a bioactive fragment thereof in the plant cell, plant tissue, plant part or plant;
or, comprising hybridizing a plant expressing the PPO polypeptide or a bioactive fragment thereof with another plant, and screening of a plant or a part thereof capable of gaining or improving the tolerance to a PPO-inhibiting herbicide;
or, comprising gene editing a protein with PPO activity of the plant cell, plant tissue, plant part or plant to achieve expression of the PPO polypeptide or a bioactive fragment thereof.

The present invention also provides use of the PPO polypeptide or a bioactive fragment thereof or the polynucleotide for gaining or improving tolerance of a host cell, plant cell, plant issue, plant part or plant to a PPO-inhibiting herbicide.

In one embodiment, the host cell is a bacterial cell or a fungal cell.

The herbicide-resistant PPO protein may be obtained from a natural source by extraction and purification using methods widely known in the art. Alternatively, it may be obtained as a synthetic protein prepared by chemical synthesis, or as a recombinant protein prepared by a genetic recombination technology. When chemically synthesized, the protein may be obtained by a polypeptide synthesis method widely known in the art. When the genetic recombination technology is used, the nucleic acid encoding the herbicide-resistant PPO protein is inserted into a proper expression vector, this vector is transformed into a host cell, the host cell is cultured to express the desired protein, and then the herbicide-resistant PPO protein is recovered from the host cell. After the protein is expressed in a selected host cell, general biochemical separation techniques, for example, treatment with a protein precipitating agent (salting out), centrifugation, ultrasonic disruption, ultrafiltration, dialysis, chromatography such as molecular sieve chromatography (gel filtration), adsorption chromatography, ion exchange chromatography, affinity chromatography and the like may be used for the isolation and purification thereof. Generally, in order to separate the protein with a high purity, these methods may be used in combination.

The herbicide-resistant PPO nucleic acid molecule may be isolated or prepared using standard molecular biological techniques, for example, a chemical synthesis or recombination method. Alternatively, commercially available one may be used.

The PPO protein provided herein may be introduced into a plant, thereby being used for enhancement of the herbicide resistance of the plant.

The herbicide-resistant PPO gene provided herein may be introduced into the plant by various methods known in the art, and can be transgenic or gene-edited by using an expression vector for plant transformation.

An appropriate promoter which may be included in the vector may be any promoter generally used in the art for plant transgenic or gene editing. For example, the promoter commonly used in plant transgenic or gene editing may include an SP6 promoter, a T7 promoter, a T3 promoter, a PM promoter, a corn ubiquitin promoter, a cauliflower mosaic virus (CaMV) 35S promoter, a nopaline synthase (nos) promoter, a figwort mosaic virus 35S promoter, a sugarcane bacilliform virus promoter, a commelina yellow mottle virus promoter, a light-inducible promoter from the small subunit of ribulose-1,5-bisphosphate carboxylase (ssRUBISCO), a rice cytosolic triosephosphate isomerase (TPI) promoter, an adenine phosphoribosyltransferae (APRT) promoter of Arabidopsis, an octopine synthase promoter, and a BCB (blue copper binding protein) promoter, but is not limited thereto.

Plant transgenic or gene editing vectors include a polyadenylation signal sequence causing polyadenylation of 3-terminus, and for example, it may include NOS 3′-end derived from a nopaline synthase gene of Agrobacterium tumefaciens, an octopine synthase 3′-end derived from an octopine synthase gene of Agrobacterium tumefaciens, 3′-end of protease inhibitor I or II gene of tomato or potato, a CaMVPoly A signal sequence, 3′-end of a rice α-amylase gene and 3′-end of a phaseolin gene, but is not limited thereto.

In the above-mentioned transgenic vector, a transit peptide required for targeting to chloroplasts may be linked to 5′-end of the PPO gene in order to express the herbicide-resistant PPO gene in the chloroplasts.

The vector may further include a gene encoding selectable marker as a reporter molecule, and example of the selectable marker may include antibiotics (e.g., neomycin, carbenicillin, kanamycin, spectinomycin, hygromycin, bleomycin, chloramphenicol, etc.) or herbicide (glyphosate, glufosinate, phosphinothricin, etc.)-resistant genes, but is not limited thereto.

Methods of vector transformation include introduction of the recombinant plasmid into the plant using Agrobacterium-mediated transformation, electroporation, microparticle bombardment, polyethylene glycol-mediated uptake, etc.

Plant transformation recipients in the present invention include a plant cell (containing a suspension-cultured cell), a protoplast, a callus, a hypocotyl, a seed, a cotyledon, a shoot as well as a mature plant.

Further, the scope of the transgenic or gene-edited plant includes a contemporary plant introduced with the gene as well as a clone or progeny thereof (T1 generation, T2 generation, or any subsequent generations). For example, the transgenic or gene edited plant comprising nucleotide sequence encoding the PPO polypeptide tolerant to the PPO-inhibiting herbicides provided in the present inventions, and a progeny comprising nucleotide sequence encoding the PPO polypeptide tolerant to the above mentioned PPO-inhibiting herbicides obtained by sexual and asexual reproduction, and the plant with inherited herbicide resistant characteristic is also included. The scope of the present invention also includes all mutants and variants showing the characteristics of the initial transgenic or gene-edited plant, together with all hybridization and fusion products of the above-mentioned transgenic or gene-edited plant. Furthermore, the scope of the present invention also includes a part of the plant, such as a seed, a flower, a stem, a fruit, a leaf, a root, a tuber, a tuberous root, which is originated from a plant which is modified by transgenic or gene editing in advance by the method of the present invention, or a progeny thereof, and is composed of at least a part of the cells modified by transgenes or gene editing.

The present invention also provides a method for controlling weeds in a plant cultivation site, comprising applying to the cultivation site a herbicidally effective amount of PPO-inhibiting herbicide, wherein the plant comprises the aforementioned plant or a plant produced by the aforementioned method.

In one embodiment, one PPO-inhibiting herbicide is used to control weeds.

In another embodiment, two or more PPO-inhibiting herbicides are used in sequence or in the mean time to control weeds.

In another embodiment, the PPO-inhibiting herbicide is applied in combination with one or more additional herbicides.

In the present invention, the term “cultivation site” includes a site in which plants of the present invention are cultivated, such as soil, and also includes, for example, plant seeds, plant seedlings, and grown plants. The term “herbicidally effective amount” means the amount of an herbicide that is enough to affect the growth or development of target weeds, for example, preventing or inhibiting the growth or development of the target weeds, or killing the weeds. Advantageously, said herbicidally effective amount does not significantly affect the growth and/or development of a plant seed, plant seedling or plant of the present invention. A person skilled in the art can determine such a herbicidally effective amount through conventional experiments.

This invention may be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that, although the terms “first,” “second,” “third” etc. may be used herein to describe various elements or components, these elements or components should not be limited by these terms. These terms are only used to distinguish one element or component from another element or component.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, elements, and/or components, but do not preclude the presence or addition of one or more other features, elements or components, and/or groups thereof. As used herein, the term “and/or” includes any and all combinations of one or more of the associated listed items.

While the invention has been described in detail in connection with a number of embodiments, the invention is not limited to such disclosed embodiments. Rather, the invention can be modified to incorporate any number of variations, alterations, substitutions or equivalent arrangements not heretofore described, but which are commensurate with the scope of the invention.

The beneficial effects of the present invention are: the mutation forms can reduce the inhibition effect of PPO-inhibiting herbicides against PPO with mutation forms, but at the same time, these mutants do not reduce the catalytic activity of PPO itself. The resistance of plants to PPO-inhibiting herbicides can be greatly improved by modifying the endogenous PPO into these mutation forms by gene editing or introducing genes with such PPO mutation forms into plants by transgenic means. Such PPO mutation forms can be used in plants including commercial crops according to the herbicide resistance characteristics and herbicide selectivity, so as to control weed growth economically.

The present invention will be further described in conjunction with the examples below. All of the methods and operations described in the examples are provided by way of exemplifications and should not be construed as limitation.

### Example 1

### Example 1: Alignment of Amino Acid Sequences of PPO from Plants

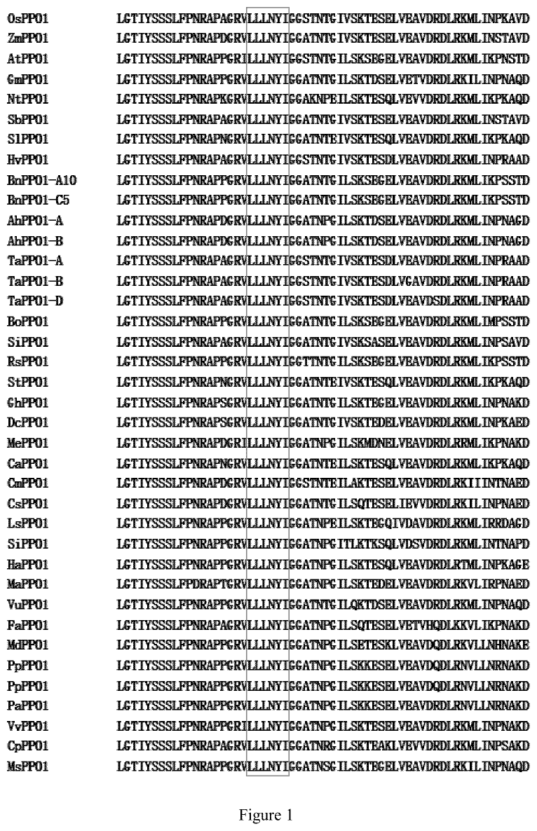

PPO, existing in animals, plants, bacteria and fungi, catalyzes protoporphyrinogen IX into protoporphyrin IX in the presence of molecular oxygen. PPO is the last key enzyme in the biosynthesis of tetrapyrrole with ferroheme and chlorophyll as its main synthetic products. There are two PPO isoenzymes in plants, which are located in mitochondrion and chloroplast, respectively. FIG. 1 shows the alignment of PPO amino acid sequences from different plants, including rice (NCBI NO: XM 015770568.2 (SEQ ID NO: 1)), corn (NCBI NO: NM_001112094(SEQ ID NO: 2)), oilseed rape (NCBI NO: BnPPO1-C5: XM_013841402.2(SEQ ID NO: 3); BnPPO1-A10: XM_013810914.2(SEQ ID NO: 4)), peanut (NCBI NO: AhPPO1-A: XM_025762937.2(SEQ ID NO: 5); AhPPO1-B: XM_025820369.2(SEQ ID NO: 6)), soybean (NCBI NO: XM_003535957.4(SEQ ID NO: 7)), sorghum(NCBI NO: XM_002455439.2(SEQ ID NO: 8)), wheat (NCBI NO: TaPPO1-A: XP_037432241.1(SEQ ID NO: 9); TaPPO1-B: XM_037583444.1(SEQ ID NO: 10); TaPPO1-D: (SEQ ID NO: 11)), tomato (NCBI NO: NM_001348379.1(SEQ ID NO: 12)), potato (NCBI NO: NP_001275224.1(SEQ ID NO: 13)), tobacco (NCBI NO: XM_016654498.1(SEQ ID NO: 14)), Arabidopsis thaliana (NCBI NO: AT4G01690(SEQ ID NO: 15)), upland cotton (NCBI NO: XM_016840317.1(SEQ ID NO: 16)), radish (NCBI NO: XP_018459031.1(SEQ ID NO: 7)), foxtail millet (NCBI NO: XP_004967639.1(SEQ ID NO: 18)), cabbage (NCBI NO: XM_013731605.1(SEQ ID NO: 19)), yam (NCBI NO: XP_039129342.1), cassava (NCBI NO: XM_021757904.2), pepper (NCBI NO: XM_016683798.1), squash (NCBI NO: XM_023107680.1), barley (NCBI NO: XM_045092307.1), cucumber (NCBI NO: XM_004149431.3), lettuce (NCBI NO: XM_023904577.2), sesame (NCBI NO: XM_011081162.2), sunflower (NCBI NO: XM_022132124.2), mulberry (NCBI NO: XM_010093132.2), cowpea (NCBI NO: XM_017556834.1), strawberry (NCBI NO: XM_004289391.2), apple (NCBI NO: XM_008383404.3), peach (NCBI NO: XM_007221411.2), cherry (NCBI NO: XM_021956996.1), apricot (NCBI NO: XM_034353497.1), grape vine (NCBI NO: XM_002273757.4), papaya (NCBI NO: XM_022041496.1), alfalfa (NCBI NO: XM_013613689.3), which indicates that the PPO protein motif LLLNYI is conservative between different plant species. Therefore, the biological effect of mutations at the corresponding sites of this motif may also be consistent across different species.

### Example 2

### Example 2: Cloning of Rice Proporphyrinogen Oxidase PPO1 Gene

The rice (Oryza sativa, Japonica Group) protoporphyrinogen IX oxidase (PPO) gene is located at Os01g18320 site of No. 1 chromosome. The primers NusOs-F:acgattgatgacgacgacaagATGGCGGCGGCGGCGGCG and NusOs-R:tccacgagctcccggactcTTACTTGTACGCATACTTGGTC were designed and synthesized according to its cDNA sequence and the vector pET-44a sequence. The cDNA of wild-type rice was used as template and Kod DNA ploymerase was used for PCR amplification. The amplification was carried out under the following conditions: 98° C. for 2 minutes; then 98° C. for 20 seconds, 65° C. for 30 seconds, and 68° C. for 60 seconds, 35 cycles; and at last 68° C. for 5 minutes. The amplified fragment was shown as 1.6 Kb in agarose gel electrophoresis and the DNA concentration thereof was determined by ultraviolet absorption after recovery.

The pET-44a (Novagen) plasmid was digested by PshAI (NEB, New England Biolabs, Boston, USA) at 37° C. for 1 hour, and then heated to 65° C. to inactivate PshAI. Equal amounts of OsPPO1DNA fragment and PshAI linearized pET-44a vector were mixed, then equal volumes of 2×Gibson Assembly Master Mix (Hanbio, Shanghai, China) were added. After mixing, the homogeneous mixture was incubated at 50° C. for one hour. 5 μl of the ligation product was used to transform the competent Escherichia coli DH5a; the bacterial solution was spreaded to the surface of an LB solid medium plate containing 100 ppm of ampicillin and cultured overnight at 37° C. On the next day, individual clones were selected and the correct clones were confirmed by individual bacterial colony PCR, after that, three correct clones were cultivated overnight at 37° C., and sufficient plasmid DNA was extracted and sent to Qingke Biotechnology Co., Ltd. (Beijing, China) for Sanger sequencing. The sequencing primers used were NUS-F: GCTGCTGCGAAATTTGAACG and NUS-R: TACAGCTGTGCGGCCGCAAG. The sequencing result proved that the correct full-length rice OsPPO1 coding region DNA could be obtained, and the expression vector of the expressed wile-type rice PPO was named as pET44a-OsPPO1 WT.

The tolerance of rice OsPPO1 to herbicides was tested by using PPO-deficient Escherichia coli (ΔhemG). ΔhemG strain is an E. coli strain lacking the hemG-type PPO gene and having kanamycin tolerance (Watanabe N, Che F S, Iwano M, et al. Dual Targeting of Spinach Protoporphyrinogen Oxidase II to Mitochondria and Chloroplasts by Alternative Use of Two In-frame Initiation Codons[J]. Journal of Biological Chemistry, 2001, 276(23):20474-20481.). The cloned rice OsPPO1 plasmid prepared above was transfected into competent cells of ΔhemG, and the PPO activity of the knockout bacteria was recovered by electrotransformation. The rice OsPPO1 plasmid could be grown on LB AGAR medium supplemented with ampicillin and kanamycin.

In order to verify whether this system could be used for evolutionary screening of rice PPO1 gene tolerance to compound A, a complementary strain of wild-type rice pET44a-OsPPO1 WT was used to test the growth difference of complementary strain on the plate containing PPO-inhibiting herbicides. Clones of the transformed complementary strains ΔhemG/pET44a and ΔhemG/pET44a-OsPPO1 WT were selected and resuspended in 100 ul LB medium. The diluted solution was then diluted again for four consecutive times with a coefficient of one-tenth. Then, 3 μl of each diluted solution was added onto LB agar medium (culture dish) containing compound A at concentrations of 0 nM, 300 nM and 1000 nM. The LB agar medium was cultured at 28° C. and the growth inhibition was assessed after 40 to 48 hours of cultivation.

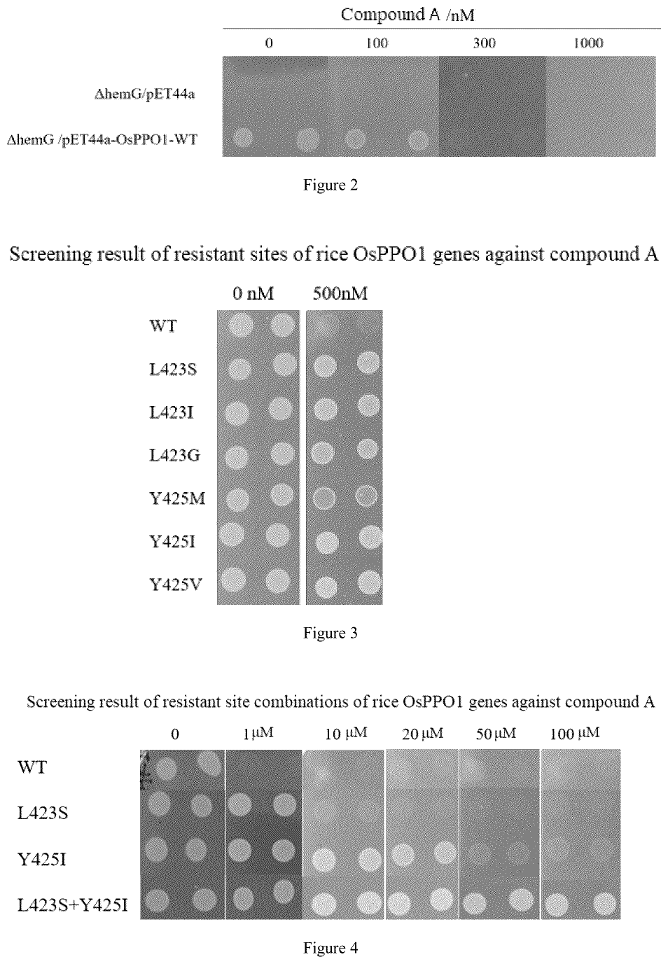

As shown in FIG. 2, on culture medium without herbicide, the ΔhemG/pET44a complementary strain did not grow while the complementary strain of the transformed rice ΔhemG/pET44a-OsPPO1 WT was able to grow normally, which indicated that the complemented OsPPO1 could perform normal PPO functions in defective E. coli.

It can also be seen that the growth of wild type rice ΔhemG/pET44a-OsPPO1 WT complementary strain was inhibited at 300 nM and no clone grew on the plate in the medium containing different concentrations of compound A. It was also demonstrated that this system could be used for evolutionary screening of rice OsPPO1 gene tolerance to compound A.

### Example 3

### Example 3: Using PPO-Deficient E. coli (ΔhemG) to Screen the Site of Rice OsPPO1 Tolerance to Compound a

In order to screen out the sites of rice OsPPO1 gene tolerance to compound A, a saturated mutation of amino acid was carried out at the sites with motif LLLNYI in rice according to the alignment result of PPO amino acid from different plants in Example 1. This was achieved by the PCR amplification of a primer containing the desired mutation of changing amino acid coding sequence into NNK and another suitable conventional primer. In NNK, N represented A/T/G/C and K represented G/T, and the NNK codon could encode any one of the 20 amino acids or stop condons. Accordingly, this was a saturated saturation mutagenesis. please see: Kille S, Acevedo-Rocha C G, Parra L P, Zhang Z G, Opperman D J, Reetz M T, Acevedo J P (2013) Reducing codon redundancy and screening effort of combinatorial protein libraries created by saturation mutagenesis. ACS Synth Biol 2(2):83-92; Directed Evolution Library Creation: methods and protocols 2nd ed. Edited by Elizabeth M. J. Gillam, Janine N. Copp and David F. Ackerley New York, NY United States: Springer, 2014.doi:10.1007/978-1-4939-1053-3. A large number of mutants would be produced. The constructed plasmids of saturation libraries with different sites were transformed into ΔhemG competent cells and the screening test of the tolerance of rice PPO1 gene different sites to compound A was conducted by using E. coli screening system in Example 2, then the normally-growing resistant clones were selected from the plate containing compound A and the genotypes thereof were identified. Six single amino acid mutants were screened out, which respectively were L423S (SEQ ID NO: 20), L423I(SEQ ID NO: 21), L423G(SEQ ID NO: 22), Y425M(SEQ ID NO: 23), Y425I(SEQ ID NO: 24) and Y425V(SEQ ID NO: 25). Compared with the wild type, these resistant mutants grew normally on LB medium containing 500 nM compound A, as shown in FIG. 3.

Specific experimental methods:

1. PCR amplification was carried out by using Kod DNA polymerase with synthesized OsPPO1-423-F and OsPPO1-423-R as primer and pET44a-OsPPO1 WT plasmid prepared in Example 2 as template. The amplification was carried out under the following conditions: 98° C. for 3 minutes; 98° C. for 20 seconds, 65° C. for 30 seconds, and 72° C. for 3 minutes, 35 cycles; and 72° C. for 5 minutes. After detection by agarose gel electrophoresis, the bands with correct size (about 9 KB) were recovered and the concentrations were determined by ultraviolet absorption.

2. 5 μl of recovery product was added to an equal volume of 2×Gibson Assembly Master Mix (Hanbio, Shanghai, China), and incubated at 50° C. for one hour after mixing well; 5 μl of the ligation product was used to transform the competent Escherichia coli DH5a, and the bacterial solution was spreaded onto the surface of an LB solid medium plate containing 100 ppm of ampicillin, and cultured overnight at 37° C. All the clones (colonies) on the plate were scrapped, plasmids were extracted, and the DNAs were quantified by UV absorption.

3. 100 ng of the constructed plasmid was transformed into ΔhemG competent cells, an LB medium plate containing 500 nM compound A was spreaded, and the cultivation was performed overnight. The normally-growing resistant clones were selected from the plate containing compound A and the genotypes thereof were identified.

TABLE 1
Primers used to prepare rice PPO mutants

| Name of the primer | Sequence of the primer (5′-3′) |
|---|---|
| NusOs-F | acgattgatgacgacgacaagATGGCGGCGGCGGCGGCG |
| NusOs-R | tccacgagctcccggactcTTACTTGTACGCATACTTGGTC |
| OsPPO1-421-F | CCGGCGGGTCGTGTTNNKCTGCTGAACTATATC |
| OsPPO1-421-R | AACACGACCCGCCGGCGCAC |
| OsPPO1-422-F | CCGGCGGGTCGTGTTCTGNNKCTGAACTATATCGGC |
| OsPPO1-422-R | CAGAACACGACCCGCCGGCGCACGG |
| OsPPO1-423-F | GGTCGTGTTCTGCTGNNKAACTATATCGGCGGTAG |
| OsPPO1-423-R | CAGCAGAACACGACCCGCCGGCGCACG |
| OsPPO1-424-F | TCGTGTTCTGCTGCTGNNKTATATCGGCGGT |
| OsPPO1-424-R | CAGCAGCAGAACACGACCCGCCGGCGC |
| OsPPO1-425-F | GTTCTGCTGCTGAACNNKATCGGCGGTAGCACC |
| OsPPO1-425-R | GTTCAGCAGCAGAACACGACCCGCCG |
| OsPPO1-426-F | CTGCTGCTGAACTATNNKGGCGGTAGCACCAA |
| OsPPO1-426-R | ATAGTTCAGCAGCAGAACACGACCCGC |
| OsPPO1-423S/425I-F | GGTGTTACTTTCCAACATCATAGGAGGTTCTACAAAT |
| OsPPO1-423S/425I-R | GTATTTGTAGAACCTCCTATGATGTTGGAAAGTAACACC |

### Example 4

### Example 4: Verifying the Herbicide Tolerance of Rice OsPPO1 Resistance Site Combination by Using the PPO-Deficient E. coli (ΔhemG)

To further enhance the tolerance of rice OsPPO1 to PPO-inhibiting herbicides, the screened single mutants L423S and Y425I with tolerance to compound A were preferably selected and combined, and the tolerance to herbicides thereof was tested by using the E. coli screening system. It can be seen that the L423S/Y425I site combination (SEQ ID NO: 26) also showed tolerance to compound A. The screened single sites or site combinations were cultured on plates containing compound A at different concentrations of 0 μM, 1 μM, 10 μM, 20 μM, 50 μM and 100 μM, and the growth inhibition thereof was observed. The screening results were shown in FIG. 4, showing significant inhibition of growth occurred at certain site combinations as the concentration of compound A increased, but the mutant site combination L423S/Y425I showed higher tolerance and also normal growth with the treatment of compound A at the concentration of 100 μM. It indicated that the mutant site L423S/Y425I combination improved rice tolerance to herbicide compound A as compared with the single mutants L423S and Y425I.

Specific experimental methods:

1. PCR amplification was carried out by using Kod DNA polymerase with the synthesized OsPPO1-423S/425I-F and OsPPO1-423S/425I-R as primer and the pET44a-OsPPO1 WT plasmid prepared in Example 2 as template. The amplification was carried out under the following conditions: 98° C. for 3 minutes; 98° C. for 20 seconds, 65° C. for 30 seconds, and 72° C. for 3 minutes, 35 cycles; and 72° C. for 5 minutes. After detection by agarose gel electrophoresis, the bands with correct size (about 9 KB) were recovered and the concentrations were determined by ultraviolet absorption.

2. 5 μl of recovery product was added to an equal volume of 2×Gibson Assembly Master Mix (Hanbio, Shanghai, China), and incubated at 50° C. for one hour after mixing well; 5 μl of the ligation product was used to transform the competent Escherichia coli DH5a, and the bacterial solution was coated to the surface of an LB solid medium plate containing 100 ppm of ampicillin, and cultured overnight at 37° C. All the clones (colonies) on the plate were scrapped, plasmids were extracted, and the DNAs were quantified by UV absorption.

3. 100 ng of the constructed plasmid was transformed into ΔhemG competent cells, an LB medium plate containing 500 nM compound A was spreaded, and the cultivation was performed overnight. The growth inhibition was observed.

4. Clones of the transformed complementary strains ΔhemG/pET44a-OsPPO1 WT, ΔhemG/pET44a-OsPPO1 L423S, ΔhemG/pET44a-OsPPO1 Y425I and ΔhemG/pET44a-OsPPO1 L423S/Y425I were selected and resuspended in an 100 ul LB medium, and the diluted solution was then diluted again for four consecutive times with a coefficient of one-tenth. Then, 3 μl of each diluted solution was added onto LB agar medium (culture dish) containing compound A at concentrations of 0 nM, 1 nM, 10 nM, 20 nM, 50 nM and 100 nM. The LB agar medium was cultured at 28° C. and the growth inhibition was assessed after 40 to 48 hours of cultivation.

### Example 5

### Example 5: Verifying the Tolerance of a Mutant LLLNYI Protein Motif in Corn ZmPPO1 to Compound a

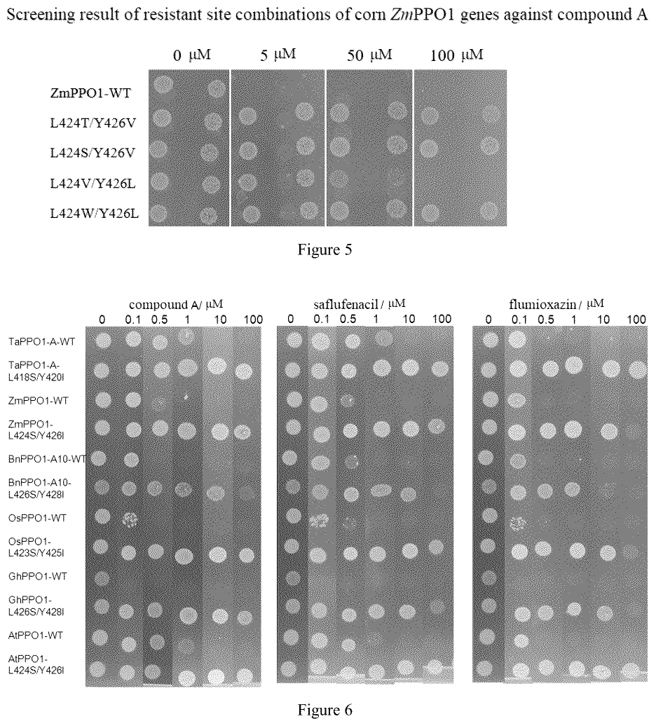

To verify whether the mutation of the PPO1-conserved protein motif LLLNYI in other plants could also confer resistance to herbicides, the mutation combination of leucine residues at the third position and tyrosine residues at the fifth position in the protein motif LLLNYI was carried out by using the same method as described in the above examples and screened by using of the LB medium containing herbicidal compound A, and the growth inhibition was observed. As shown in FIG. 5, compared with wild-type ZmPPO1-WT (SEQ ID NO: 2), the mutation combinations of leucine residues at the third position and tyrosine residues at the fifth position in the protein motif LLLNYI, comprising L424T/Y426V (SEQ ID NO: 27), L424S/Y426V (SEQ ID NO: 28), L424V/Y426L (SEQ ID NO: 29) and L424W/Y426L (SEQ ID NO: 30), grew normally without an inhibition on plates containing compound A at the concentration of 5 μM. The majority thereof showed a high tolerance with the increased concentration of compound A, indicating that tolerance to herbicides conferred by the mutation at the corresponding site of the PPO-conserved protein motif LLLNYI in different plants also had the consistent effects.

### Example 6

### Example 6: Verifying the Tolerance of LLLNYI Protein Motif Mutation to Compound a and Other PPO-Inhibiting Herbicides in PPO1 of Other Crops

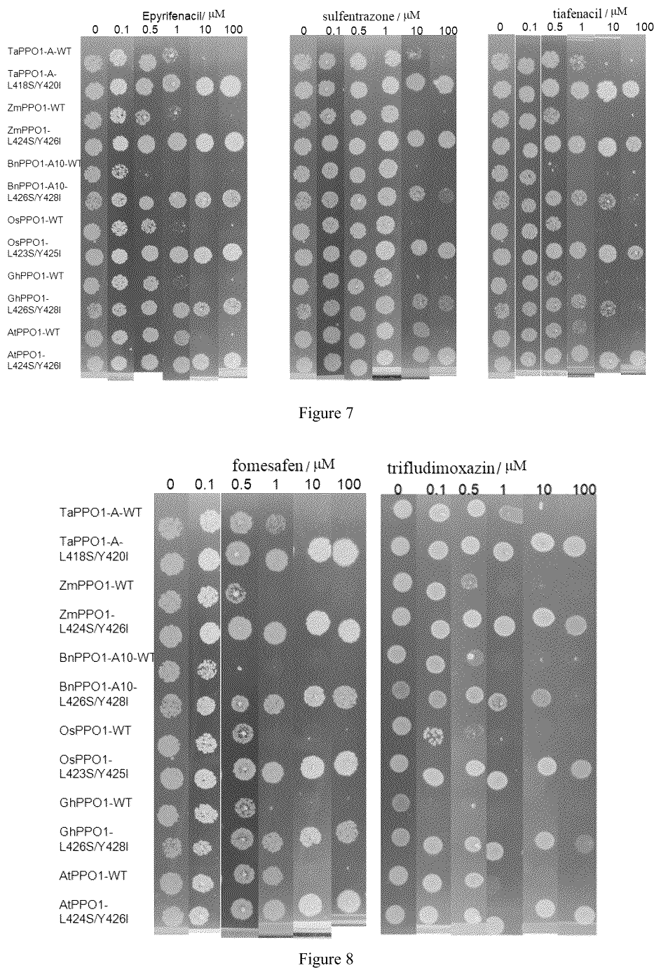

To further verify the effect on the tolerance to herbicides conferred by a mutation at the corresponding site of the PPO-conserved protein motif LLLNYI in other crops, the vector containing the expressed genes comprising OsPPO1 WT (SEQ ID NO: 1) of wild-type rice PPO1, OsPPO1 L423S/Y425I (SEQ ID NO: 26) of mutant rice PPO1, ZmPPO1WT (SEQ ID NO: 2) of wild-type corn PPO1, ZmPPO1 L424S/Y426I (SEQ ID NO: 31) of mutant corn PPO1, TaPPO1-A WT (SEQ ID NO: 9) of wild-type wheat PPO1, TaPPO1-A L418S/Y420I (SEQ ID NO: 38) of mutant wheat PPO1, BnPPO1-A10 WT (SEQ ID NO: 4) of wild-type oilseed rape PPO1, BnPPO1-A10 L423S/Y425I (SEQ ID NO: 33) of mutant oilseed rape PPO1, GhPPO1 WT (SEQ ID NO: 16) of wild-type cotton PPO1, GhPPO1 L426S/Y428I (SEQ ID NO: 45) of mutant cotton PPO1, AtPPO1 WT (SEQ ID NO: 15) of wild-type Arabidopsis thaliana PPO1 and AtPPO1 L423S/Y425I (SEQ ID NO: 44) of mutant Arabidopsis PPO1, was constructed and transformed into PPO-deficient Escherichia coli (ΔhemG) for complementary by using the same method described in Example 3 and Example 4 and the primers as shown in Table 2. Clones of PPO-deficient Escherichia coli (ΔhemG) transformed by wild-type PPO1 genes or various mutant PPO1 genes in different crops were selected and resuspended in an 100 ul LB medium, and the diluted solution was then diluted again for two times with a coefficient of one-tenth. Then, 3 μl of each diluted solution was added onto LB agar medium (culture dish) containing respectively compound A at concentrations of 0.1 μM, 0.5 μM, 1 μm, 10 μM and 100 μM, saflufenacil at concentrations of 0.1 μM, 0.5 μM, 1 μM, 10 μM and 100 μM, flumioxazin at concentrations of 0.1 μM, 0.5 μM, 1 μM, 10 μM and 100 μM, epyrifenacil at concentrations of 0.1 μM, 0.5 μM, 1 μM, 10 μM and 100 μM, sulfentrazone at concentrations of 0.1 μM, 0.5 μM, 1 μM, 10 μM and 100 μM, tiafenacil at concentrations of 0.1 μM, 0.5 μM, 1 μM, 10 μM and 100 μM, fomesafen at concentrations of 0.1 μM, 0.5 μM, 1 μM, 10 μM and 100 μM and trifludimoxazin at concentrations of 0.1 μM, 0.5 μM, 1 μM, 10 μM and 100 μM. The LB agar medium was cultured at 28° C. in thermostatic incubator and the growth inhibition was assessed after 40 to 48 hours of cultivation. The results were shown in FIGS. 6-8, indicating that the resistance sites or combinations in examples 3 and 4 were also tolerant to other PPO herbicides.

TABLE 2
List of primers

| Name of the primer | Sequence of the primer (5′-3′) |
|---|---|
| NusOs-F | acgattgatgacgacgacaagATGGCGGCGGCGGCGGCG |
| NusOs-R | tccacgagctcccggactcTTACTTGTACGCATACTTGGTC |
| OsPPO1-423S/425I-F | GGTGTTACTTTCCAACATCATAGGAGGTTCTACAAAT |
| OsPPO1-423S/425I-R | GTATTTGTAGAACCTCCTATGATGTTGGAAAGTAACACC |
| TaPPO1A-F | ccgcgcggcagccatATGGCCGGCGCAACAATG |
| TaPPO1A-R | tttgttagcagccggatcTCACTTGTAGGCATACTTGGTC |
| TaPPO1-418S/420I-F | GGAAGAGTGTTACTTtcGAACatTATCGGGGGTTC |
| TaPPO1-418S/420I-R | AAGTAACACTCTTCCAGCAGGAGCACG |
| ZmPPO1-T38F | ccgcgcggcagccatatggctgctgtggcgggggcg |
| ZmPPO1-R | tttgttagcagccggatctcacttgtaggcatacttggtcaag |
| ZmPPO1-424S/426I-F | ggtagggtgttacttAGCaacATTataggaggtgct |
| ZmPPO1-424S/426I-R | aagtaacaccctaccgtcaggagcacg |
| AtPPO1-F | tgccgcgcggcagccatatgTCagtggccggtggaccaac |
| AtPPO1-R | tttgttagcagccggatcttacttgtaagcgtaccgtgacatg |
| AtPPO1-423S/425I-F | ggaagaattttgctgAGCaacATTattgggggtctacaaac |
| AtPPO1-423S/425I-R | cagcaaaattcttccgggcggtgc |
| GhPPO1-F | TGCCGCGCGGCAGCCATatgacggctctaatcgacc |
| GhPPO1-R | GCTTTGTTAGCAGCCGGATCCttatttgtatgcatattgtg |
| GhPPO1-426S/428I-F | ggcagggtgttgctctCgaacATCataggaggag |
| GhPPO1-426S/428I-R | gagcaacaccctgccagatggagctcg |
| BnPPO1-A10-F | cgcgcggcagccatATGGATTTCTCTCTTCTCCGTCCGGC |
| BnPPO1-A10-R | gttagcagccggatcTTACTTGTAAGCATACCTTGACAT |
| BnPPO1-A10- | GGAAGAGTGTTGCTATcGAACatCATCGGTGGAGCTAC |
| 426S/428I-F | |
| BnPPO1-A10- | TAGCAACACTCTTCCAGGTGGTGCT |
| 426S/428I-R | |

### Example 7

### Example 7: Verifying the Tolerance of the Resistance Sites or Combinations of Rice OsPPO1 to Different Types of PPO Herbicides

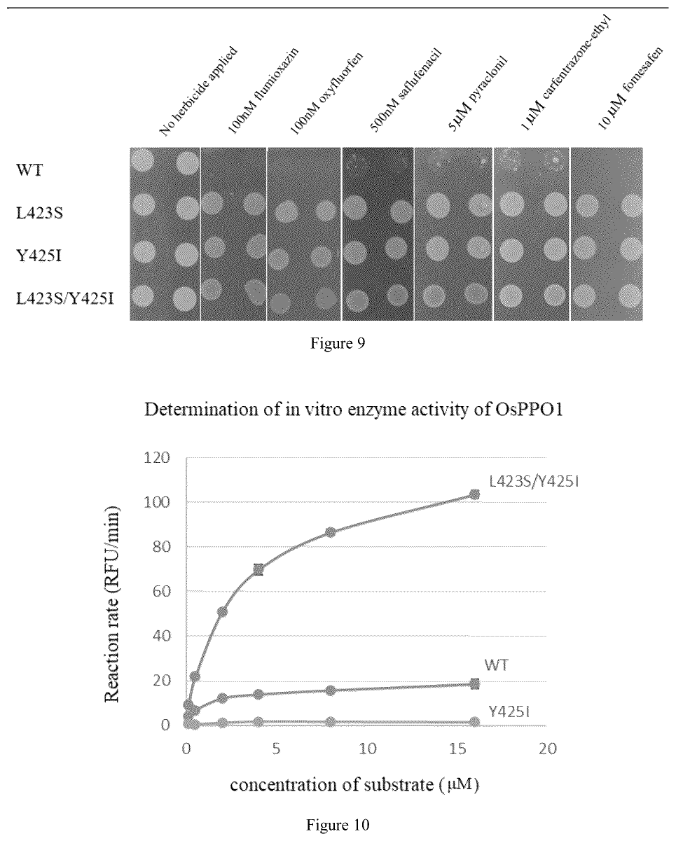

To verify whether the resistance sites or combinations in examples 3 and 4 were also tolerant to other PPO herbicides, some sites or combinations were preferably selected to verify the tolerance thereof to different types of PPO herbicides. Clones of PPO-deficient Escherichia coli (ΔhemG) transformants transformed by wild-type (WT) OsPPO1 genes or various mutant OsPPO1 genes were selected and resuspended in an 100 ul LB medium, and the diluted solution was then diluted again for two consecutive times with a coefficient of one-tenth. Then, 3 of each diluted solution was added onto LB agar medium (culture dish) containing respectively flumioxazin at the concentration of 100 nM, oxyfluorfen at the concentration of 100 nM, saflufenacil at the concentration of 500 nM, pyraclonil at the concentration of 5 μM, carfentrazone-ethyl at the concentration of 1 μM and fomesafen at the concentration of 10 μM. The LB agar medium was cultured at 28° C. in thermostatic incubator and the growth inhibition was assessed after 40 to 48 hours of cultivation. The results were shown in FIGS. 9, indicating that the resistance sites or combinations in examples 3 and 4 were also tolerant to other PPO herbicides.

### Example 8

### Example 8: An In Vitro Enzyme Activity and Resistance Test of Resistance Site Combination Proteins (Polypeptides) of Rice OsPPO1

### 1. Preparation of Protoporphyrinogen

Protoporphyrinogen, a substrate catalyzed by PPO, was prepared by reducing protoporphyrin with sodium amalgam. 10 mg of protoporphyrin was dissolved in 10 mL solvent, added with 20% sodium amalgam at the rate of 0.2 g/mL, reacted for 2 hours, then filtered under the protection of nitrogen in dark place. After the reaction was completed, the reaction solution should become achromatic or light brown. The reaction solution was diluted by adding with reaction buffer (100 mM Tris-HCl, 1 mM EDTA, 5 mM DTT, 0.1% Tween 20/80), and 10% hydrochloric acid was used to adjust pH to about 8.0. Protoporphyrinogen at an approximate concentration of 100 μM was finally obtained, subpackaged and stored with liquid nitrogen or in a −80° C. temperature.

### 2. Expression and Purification of OsPPO1 Proteins

1) PCR amplification was carried out by using Kod DNA polymerase with pET44a-OsPPO1-WT and screening mutants as template and 28MBP-OsPPO1-T38F: CCGCGCGGCAGCCATATGGCGGGTTCTGGTACGATTG and 28MBP-OsPPO1-T38Rn: GAGCTCGAATTCGGATCCTTACTTGTACGCATACTTGGTCAG as primers. The amplification was carried out under the following conditions: 95° C. for 3 minutes; 98° C. for 10 seconds; 60° C. for 30 seconds; 68° C. for 1 minute, 35 cycles; and 68° C. for 5 minutes. After detection by agarose gel electrophoresis, the bands with correct size (about 1.5 KB) were recovered and the concentrations were determined by ultraviolet absorption.

2) 4 μl of recovery product and 1 μl of pET28a-MBP vector were added to an equal volume of 2×Gibson Assembly Master Mix (Hanbio, Shanghai, China), mixed, and incubated at 50° C. for one hour; 5 μl of the ligation product was used to transform the competent Escherichia coli DH5a, and the bacterial solution was spreaded to the surface of an LB solid medium plate containing 100 mg/L of kanamycin sulfate, and cultured overnight at 37° C. All the clones on the plate were selected and sequenced.

3) The constructed fusion expression vector pET28a-MBP-OsPPO1 was transferred into E. coli BL21 (DE3), subjected to induced-expression with 0.5 mM IPTG, then purified with the Ni-NTA column, afterwards subjected to thrombin digestion, dialysed, and purified secondly by dextrin-column and Ni-column. The specific methods were as follows. The OsPPO1 enzyme recombinant expression vector was transformed into the BL21 (DE3) cell, and the clone thereof was selected into 10 ml of LB medium and cultivated overnight with Kana resistance at 37° C. and on a shaker at 200 rpm, then transferred into a 2 L shake flask containing 1 L TB medium, cultivated on a shaker at 37° C. and 200 rpm until the OD600 reached 0.6-0.8, cooled to a temperature of 18° C., and subjected to induced-expression with 0.5 mM IPTG overnight. The strains were collected by centrifugation at 4000×g. The collected strains were re-suspended with the Ni-buffer A (50 mM Tris pH 8.0, 500 mM NaCl, 50 mM imidazole), homogenized by high pressure cell homogenizer, and centrifuged at 4000×g at 4° C. for 30 minutes; the supernatant was purified with the Ni column, then the purity thereof was detected by SDS-PAGE. The thrombin enzyme was added according to the quantity of proteins, dialyzed with 50 mM Tris pH 8.0, 500 mM NaCl, 1 mM DTT buffer. On the second day, then purified by dextrin-column and Ni column successively; and the eluates containing of the protein of interest were collected, concentrated, subpackaged and stored in a −80° C. temperature for later use.

### 3. Activity Test of OsPPO1

Determination of substrate affinity and catalytic activity of enzymes: the reaction buffer (100 mM Tris-HCl, 1 mM EDTA, 5 mM DTT, 0.1% Tween 20/80) was used to prepare the reaction solution containing the substrate protoporphyrinogen with different concentrations, and the test concentrations were 0.125, 0.5, 2, 4, 8 and 16 μM. The OsPPO1 enzyme was diluted to 10 μM and 5 μl was absorbed to a black 96-well ELISA plate, to which the reaction solution was added until the total volume reached 100 μl. The final working concentration of enzyme was 500 nM. The solution was immediately well mixed and monitored by a fluorescence microplate reader. The mix was stimulated at 410 nm and detected at 630 nm. The reaction curves were made, as shown in FIG. 10.

### Example 9

### Example 9: Homologous Replacement of Rice PPO1 Mutant Mediated by CRISPR/Cas9 to Obtain Herbicide Resistance

To obtain non-transgenic rice with herbicide resistance, the above mentioned L423S/Y425I mutation site combination was subjected to homologous replacement mediated by CRISPR/cas9. There were nine exons and eight introns in rice OsPPO1 gene, however, the two target sites L423S and Y425I were located at the eighth exon.

Design of gRNA: one gRNA was designed upstream of L423S and downstream of Y425I, respectively, and cleaved once at each site, respectively; and the DNA between two sites were simultaneously replaced by the means of homologous replacement. The sequence of rice OsPPO1 was input in http://crispor.tefor.net/crispor.py to evaluate all possible gRNAs. According to the principles that specificity scoring value was greater than 90 (Hsu P D, Scott D A, Weinstein J A, Ran F A, Konermann S, Agarwala V, Li Y, Fine E J, Wu X, Shalem O, Cradick T J, Marraffini L A, Bao G, Zhang F. Nat Biotechnol. 2013 September; 31(9):827-32. doi: 10.1038/nbt.2647. Epub 2013 Jul. 21), the off-target effect was avoided and the length was shortened as far as possible, the following two gRNAs were selected: gRNA: Osppo1 gRNA5-2:acatgaactagtaatgattgggg (top strand); and Osppo1 gRNA8-3: agcagctggagttgaaaaacagg (bottomstrand), wherein the underlined was the PAM sequence.

Design of repair template: the DNA fragment cleaved by two selected targeted RNAs was in length of 1212 bp. However, due to the close location of the sites 423 and 425, while designing the repair template, the left homology arm was in length of 1127 bp and the right homology arm was in length of 82 bp. Moreover, the digestion target sites were left at each of the left and right ends in order to cleave the repair template from the vector. So the total length of the template was 1258 bp (SEQ ID NO: 49).

Editing vector: the Osppo1 gRNA5-2 and Osppo1 gRNA8-3 were expressed respectively by rice U3 promoter. Thus, the two gRNA expression cassettes were sent together with the repair template to GenScript (Nanjing) Co., Ltd. for synthesis. The two synthesized gRNA expression cassettes and a vector pRGEB32 (Addgene #63142) were subjected to enzyme digestion with BsaI enzyme, detected by agarose gel electrophoresis, then purified and recovered, and ligated and transformed with T4DNA ligase (NEB, New England Biolabs, Boston, USA) to generate an editing vector.

Transformation by gene gun, screening, differentiation, rooting and soil cultivating seedlings: the above constructed editing vector was verified by sequencing and multi-enzyme digestion, and was then used together with the synthesized repair template NDA for rice transformation mediated by gene gun.

Specific methods of the transformation of rice callus mediated by gene gun:

1. High-quality seeds from Huaidao No. 5 and Jinjing 818 rice varieties were selected, sterilized with the solution comprising 70% alcohol and 20% sodium hypochlorite, rinsed with sterile water, and inoculated into callus induction medium. After one week of cultivation, the embryos were removed and the exfoliated callus was inoculated into callus induction medium. After two weeks, subculture was carried out for subsequent infection.

### 2. Preparation of Microprojectile and Gene Gun Transformation

(1) Preparation of gold powder suspension: 30 mg of gold powder (diameter 0.6 m) was weighted by imported 1.5 mL EP tube, to which 1 mL of 70% ethanol was added, fully vortexed, and the supernatant was abandoned by centrifugation; sterile water was added to rinse and repeated for 3 times. 500 μL of sterile glycerol (50%) was added and vortexed thoroughly, and the gold powder suspension at a concentration of 60 μg/L was prepared, then stored at −20° C.

(2) DNA wrapping: 25 μL of gold powder suspension (60 μg/μL), editing vector and repair template (1:10), 25 μL of CaCl₂)(SIGMA) (2.5 mol/L) and 10 μL of spermidine (0.1 mol/L) were added into 1.5 mL centrifuge tube successively. The above mixed sample was fully vortexed for 3-5 minutes and placed on ice for 10 minutes, and the supernatant was abandoned by centrifugation; finally, 30 μL of anhydrous ethanol was added for re-suspending and the final volume.

(3) Gene gun bombardment: the super clean bench was cleaned, the bench top was wiped with alcohol, the instrument was adjusted and the bombardment was carried out in accordance with the operation instructions. The bombardment parameter was adjusted to 27 vacuum degree, 1100 psi, 6 cm. After bombardment, the callus was cultured in darkness for 16 hours at 25° C., then the callus was transferred to the recovery medium at 25° C. for dark cultivation for one week.

(4) Screening of resistant callus and differentiation of plants: the callus was transferred to a screening medium for screening, and the medium was changed every two weeks. After four weeks of screening, samples were taken to detect whether target replacement occurred.

(5) The callus that tested positive was transferred to a differentiation medium wherein the differential culture was carried out in a light incubator at 28° C.

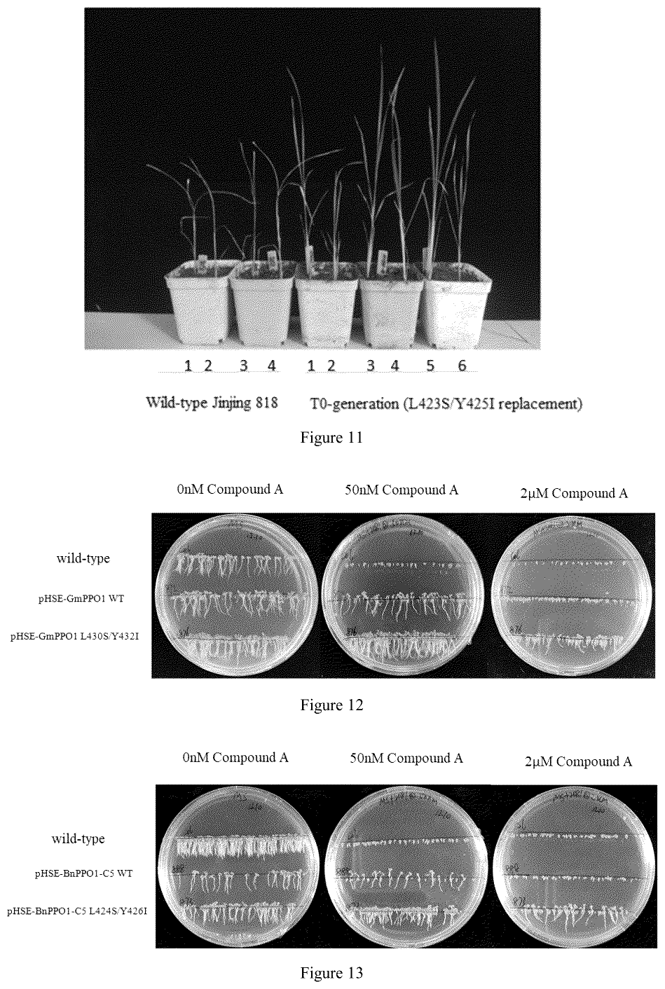

The transgenic rice genomic DNA was extracted after the resistant callus was induced into rice seedlings, and PCR amplification test was performed by using the DNA thereof as a template. The TO-generation rice line was successfully obtained by simultaneously replacement at site L423S/Y425I of the rice OsPPO1 gene. To further verify the tolerance of the obtained TO-generation rice seedlings to compound A, the rice seedlings were treated with compound A at the rate of 9 g/ha, and lines that carried homologous replacement at the site of L423S/Y425I grew normally, compared with wild-type lines which died 3 days after treatment, as shown in FIG. 11, indicating that the mutation of rice OsPPO1 gene at site L423S/Y425I conferred herbicidal compound A tolerance to the plant. Rice seedlings of T1-generation and T2-generation were obtained from the cultivation cultivated by harvesting seeds. It was proved that the obtained rice seedlings of T1-generation and T2-generation were also tolerant to compound A and the tolerance of T2-generation rice seedlings to compound A was shown in Table 3.

TABLE 3
Tolerance of T2-generation rice seedlings
to compound A (soil treatment, 13DAA)

| Test | Rate | Efficacy (Phytotoxicity) Ranking | |
|---|---|---|---|
| Product | (g a.i./ha) | T2-generation rice | Wild-type rice |
| 4.25% | 15 | 1 | 5 |
| Compound A | 30 | 2 | 7 |
| | 60 | 4 | 8 |
| | 120 | 6 | 9 |

Note:
0—No difference;
1—the plants are slightly burned;
2—the plants are significantly burned;
3—the plants are severely burned;
4—the plants are slight wilted;
5—the plants are significantly wilted;
6—the plants are severely wilted;
7—the minority die;
8—the majority die;
9—all die.

### Example 10

### Example 10: Overexpressing the Tolerance of LLLNYI Protein Motif Mutations in PPO1 from Other Crops to Compound a in Arabidopsis thaliana

In order to rapidly verify the tolerance of LLLNYI protein motif mutations in PPO1 from different crops to compound A, vectors of overexpressed wild-type and mutant PPO1 genes in rice, corn, soybean and oilseed rape were constructed respectively.

### 1. Construction of the Overexpression Vector

1) Primers: Primers were designed according to the selected restriction enzyme cutting sites and the nucleotide sequence of the gene itself to amplify the wild-type and mutant. The designed primers were synthesized by Beijing Qingke Biotechnology Co., Ltd.

| Name of the primer | Sequence of the primer (5′-3′) |
|---|---|
| 401V-OsPPO1-F | gatactcgagtaatctagaatggccgccgccgccgcag |
| 401V-OsPPO1-R | cgaacgaaagctctgagctctcacttgtaggcgtacttg |
| 401V-ZmPPO1-F | gatactcgagtaatctagaatggtcgccgccaccgcc |
| 401V-ZmPPO1-R | cgaacgaaagctctgagctctcacttgtaggcatacttg |
| 401V-GmPPO1-F | gatactcgagtaatctagaAtggtttccgtcttcaac |
| 401V-GmPPO1-R | cgaacgaaagctctgagctcctatttgtacactctatttg |
| 401V-BnA10-PPO1-F | gatactcgagtaatctagaATGGATTTCTCTCTTCTCC |
| 401V-BnA10-PPO1-R | cgaacgaaagctctgagctcTTACTTGTAAGCATACCTTG |

2) PCR amplification: The gene of interest was amplified by using the synthesized primers and Q5DNA polymerase (NEB, New England Biolabs, Boston, USA). The amplified product was detected by agarose gel electrophoresis and recovered according to the operating instructions of TIAN quick Midi Purification kit. After the completion of the recovery, the concentration of DNA extracted was determined by Nanodrop.

3) Construction of overexpression vector: an overexpression vector was constructed with the recovered PPO1 fragment and plasmid pHSE401V digested by XbaI and SacI by using the HB-in fusion™ seamless cloning kit of HanBio Biotechnology Co., Ltd., (Shanghai), and then transformed into the competent E. coli DH5α to obtain a positive clone; the positive clone was transformed into Agrobacterium for later use after verified by sequencing and restriction endonuclease digestion.

2. Transformation of Arabidopsis thaliana by Dipping Inflorescences

1) Sowing: plump and vernalized wild-type Arabidopsis thaliana seeds were selected, treated with 75% alcohol for 1 minute, disinfected with 10% NaClO for 6 minutes, and washed with sterile water for 5 to 6 times. After the sterilization was completed, the seeds were placed on a MS medium plate for one week, then transplanted to sterilized nutrient soil (nutrient soil:vermiculite=1:1) and cultured in a greenhouse at (25±2) ° C. with a photoperiod of 16h/8h (light/dark).

2) Activation and preparation of Agrobacterium: Agrobacterium strains with expression vector stored at low temperature were streaked on a resistant plate containing clarithromycin and rifampicin. Single colonies were picked out and inoculated into the 5 mL liquid LB medium to which corresponding antibiotics were added, cultured in a shaker at 28° C. and 250 rpm for 18 to 24 hours. Then the cultivation was enlarged according to 1:100 inoculation under the same conditions and the total volume of bacteria solution was 50 ml until the OD6000 was in the range of 1.0-1.5.

3) Preparation of the infestation solution: the cultured bacteria solution was centrifuged at 6000 rpm for 10 minutes, and the supernatant was discharged. The stains were resuspended in the infestation solution containing 5% sucrose until the OD6000 was about 0.8. SilwetL-77 (0.02%-0.04%) was added to the bacteria solution and well mixed.

4) Infestation of Arabidopsis thaliana inflorescences: the uninfected Arabidopsis thaliana with good growth state and lush inflorescences were selected for infection and their fruit pods were cut off from the plants by scissors prior to transformation. The Arabidopsis thaliana inflorescences were dipped in the prepared infection solution for 0.5 to 1 minute. Then the infected Arabidopsis thaliana seedlings were placed in dark and humid conditions for 24 hours. After one week, the infection was repeated.

### 5) Screening of Transgenic Lines

After the transformation of dipped Arabidopsis thaliana inflorescences, Arabidopsis thaliana TO-generation seeds were collected and sowed on a resistant MS plate containing 30 mg/L hygromycin to screen positive plants. The screened positive seedlings were transferred to a pot containing soil and placed in a greenhouse for cultivation to obtain the following: the overexpressed rice OsPPO1 L423S/Y425I and the overexpressed the OsPPO1 WT seedlings or events in Arabidopsis thaliana; the overexpressed soybean GmPPO1 L430S/Y432I and the overexpressed GmPPO1 WT seedlings or events in Arabidopsis thaliana; the overexpressed oilseed rape BnPPO1-C5 L424S/Y426I and the overexpressed BnPPO1-C5 WT seedlings or events in Arabidopsis thaliana; the overexpressed corn ZmPPO1 L424T/Y426V, ZmPPO1 L424S/Y426V, ZmPPO1 L424V/Y426L, ZmPPO1 L424W/Y426L, ZmPPO1 L424S/Y426I and the overexpressed ZmPPO1 WT seedlings or events in Arabidopsis thaliana.

### 3. Herbicide Resistance Test

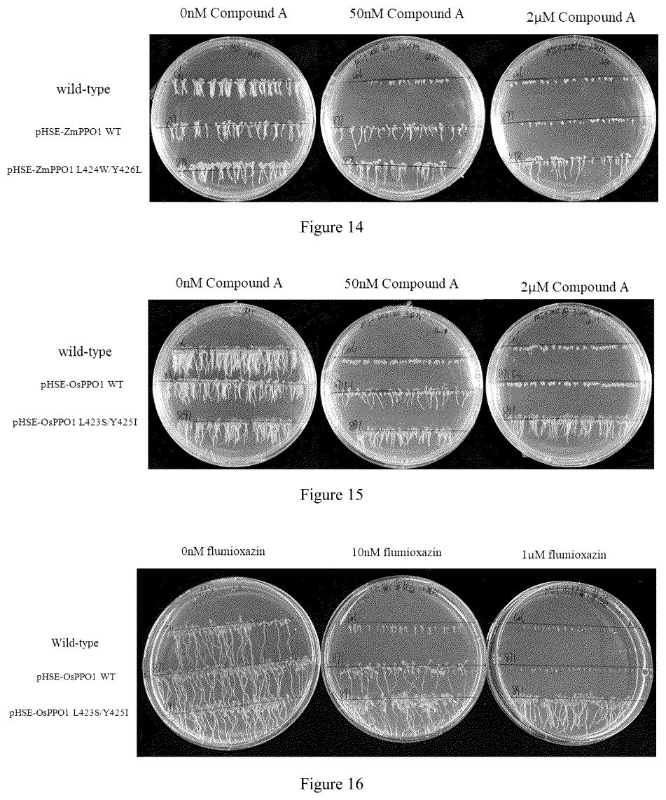

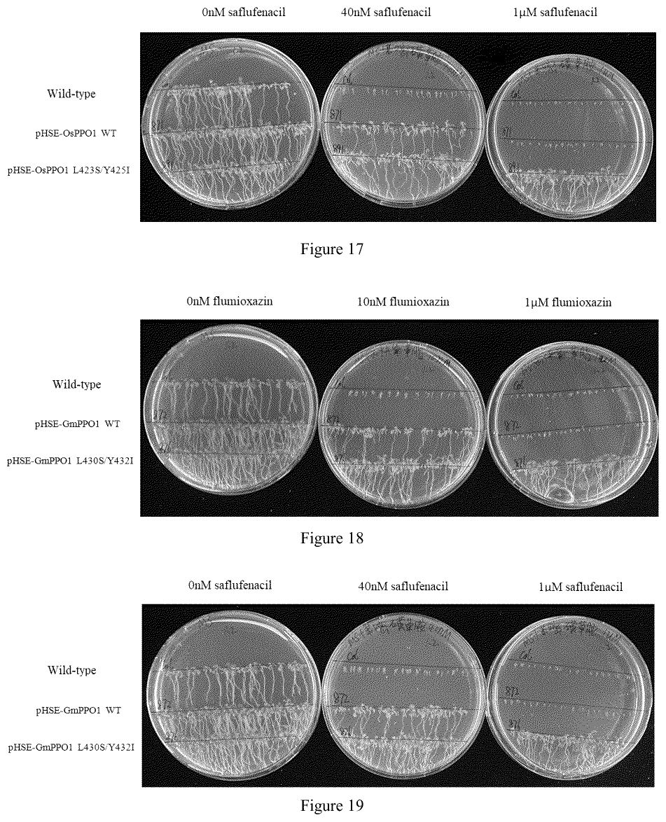

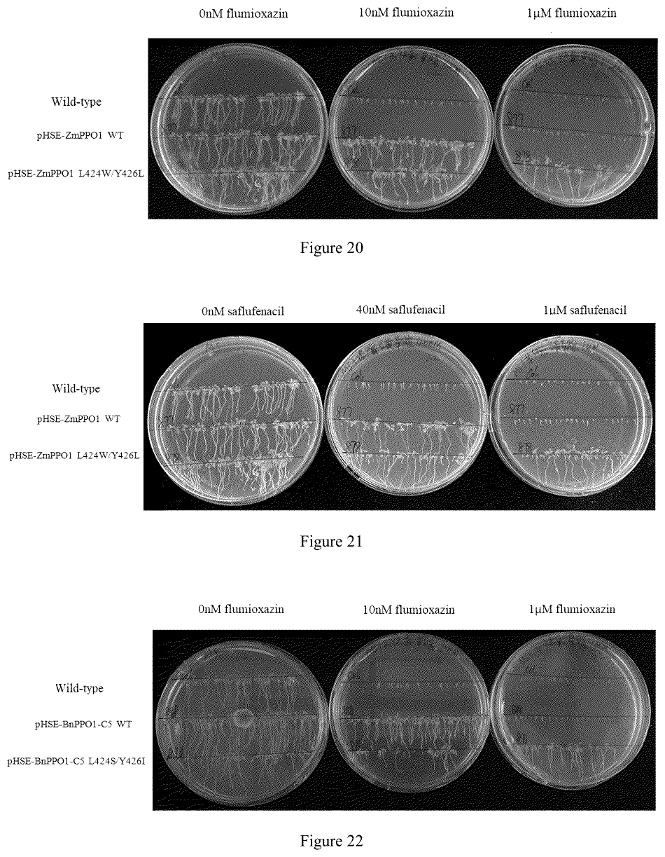

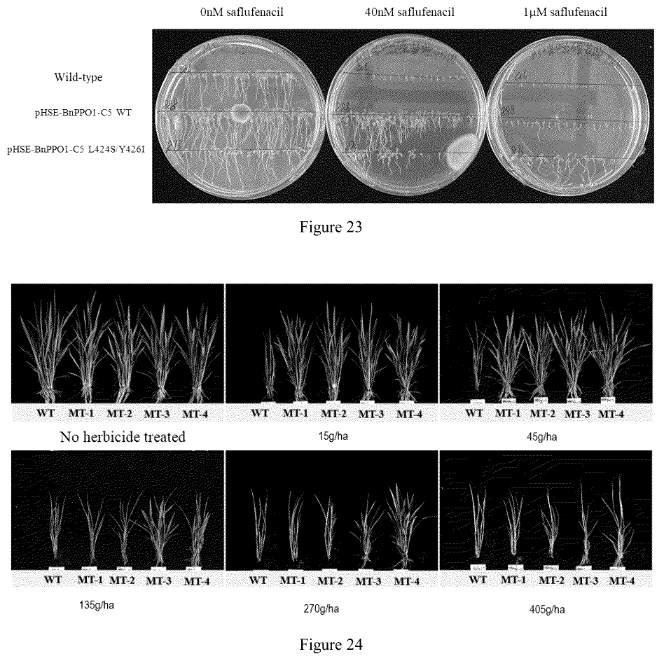

The obtained overexpressed mutant and wild-type Arabidopsis thaliana seeds of different crops were tested for resistance on a MS medium (petri dish) containing different concentrations of PPO-inhibiting herbicidal compounds, as shown in FIG. 12-23. Compared with wild-type Arabidopsis thaliana, both the overexpressed LLLNYI protein motif mutation in PPO1 from different crops and the overexpressed PPO1 WT showed certain level of tolerance/resistance to herbicides in the present invention, and their resistance levels were similar at the application concentration of 50 nM. However, at the higher application concentration of 2 μM, the overexpressed LLLNYI protein motif mutation in PPO1 from different crops still showed resistance while the overexpressed PPO1 WT from different crops showed no difference with the wild-type Arabidopsis thaliana control, indicating that after the overexpression of LLLNYI protein motif mutation in PPO1 from different crops, such crops would have a higher tolerance to PPO-inhibiting herbicidal compounds.

### Example 11

### Example 11: Overexpressing Rice OSPPO1 L423S/Y425I Mutation to Obtain Herbicide Resistance

In order to further test the tolerance of the obtained mutants in plants to compound A, the mutants L423S/Y425I screened from rice were overexpressed in rice.

### 1. Construction of the Overexpression Vector

1) Primers: primers were designed according to the selected restriction enzyme cleavage sites and the nucleotide sequence of the gene itself to amplify the mutant L423S/Y425I. The designed primers comprising PPO1-F:GCCAGTGCCAAGCTCTGCAGattcgggtcaaggcgga and PPO1-R:ACATGATTACGAATTCtctagtaacatagatgacaccgcgc were synthesized by Beijing Qingke Biotechnology Co., Ltd.

2) PCR amplification: the gene of interest was amplified by using the synthesized primers and Q5DNA polymerase (NEB, New England Biolabs, Boston, USA). The amplified product was detected by agarose gel electrophoresis and recovered according to the operating instructions of TIAN quick Midi Purification kit. After the completion of the recovery, the concentration of extracted DNAs was determined by Nanodrop.

3) Construction of rice overexpression vector: a rice overexpression vector pCAMBIA1301-OsPPO1 L423S/Y425I was constructed with the recovered PPO1 fragment and plasmid pCAMBIA1301 digested by KpnI and Hind III by using the HB-in fusion™ seamless cloning kit of HanBio Biotechnology Co., Ltd., (Shanghai), and then transformed into the competent E. coli DH5a to obtain a positive clone. The positive clone was transformed into Agrobacterium after verified by sequencing and restriction endonuclease digestion.

### 2. Agrobacterium-Mediated Transformation of Rice Callus and Occurrence of Transgenic Events:

1) 100 ng of vector plasmid of rice overexpression vector pCAMBIA1301-OsPPO1 L423S/Y425I and pCAMBIA1301-OsPPO1 WT was aspirated and added in the competent Agrobacterium EH105, respectively, placed on ice for 5 minutes, rapidly frozed by immersing in liquid nitrogen for 5 minutes, fished out and stood at 37° C. for 5 minutes and finally placed on ice for 5 minutes; to which 500 μl of YEB solution culture (antibiotic-free) was added, and cultivated on a shaker at 28° C. and 200 rpm/minute for 2-3 hours; the colonies were collected by centrifugation at 3500 rpm/minute, and the collected cells were coated on the YEB (clarithromycin+rifampicin) plate and cultivated for 2 days in a 28° C. incubator; the single clones were picked out, cultivated in liquid medium and stored at −80° C. to keep the life of bacteria. 2) Cultivation of Agrobacterium: the transformed Agrobacterium single clones were picked out and cultured in YEB liquid medium (clarithromycin+rifampicin) in a shaker at 28° C. until the OD600 was 0.5, the colonies were collected at 3500 rpm, diluted with equal amount of AAM (1 ml AAM+1 μl 1000×AS) liquid medium to infect the callus.

3) Induction of callus from Huaidao No. 5 rice variety: prior to the preparation of Agrobacterium, rice callus was prepared at first. Rice seeds were peeled, washed with sterile water as many times as needed until the washing water became clear. Then the seeds were disinfected with 70% alcohol for 30 seconds and then with 5% sodium hypochlorite. The seeds were cultivated in a horizontal shaker for 20 minutes, disinfected with sodium hypochlorite and washed with sterile water for 5 times, placed on a sterile absorbent paper to air-dry the surface moisture of the seeds, and inoculated in an induction medium to cultivate the callus at 28° C.

4) Infection of rice callus with Agrobacterium: the Huaidao No. 5 callus with a diameter of 3 mm was selected for sub-cultivation for 10 days and the callus was collected into a 50 ml centrifuge tube. A bacterial solution of Agrobacterium with a modulated concentration was added into the centrifuge tube containing the callus, and the centrifuge tube was placed on a shaker at 28° C. and 200 rpm to infect for 20 minutes; after the infection was completed, the bacterial solution was discharged, and the callus was placed on a sterile filter paper and air-dried for about 20 minutes and co-cultivated on a co-culture plate on which an sterile filter paper wetted with an AAM (1 ml AAM+30 μl 1000×AS) liquid culture was covered; after 3 days of infestation, Agrobacterium was washed and removed (i.e., washed with sterile water for 5 times and then washed with 500 mg/L of cephalosporin antibiotic for 20 minutes), and then the callus was screened and cultivated in 50 mg/L hygromycin screening medium.

5) Screening, differentiation and rooting of resistant callus: the co-cultured callus was transferred to screening medium for the first round of screening (2 weeks); after the first round of screening was completed, the newly grown callus was transferred to screening medium (containing 50 mg/L hygromycin) for the second round of screening (2 weeks); after the screening was completed, yellow-white callus with a good growth state were picked out for differentiation, and seedlings of about 1 cm were obtained after 3 to 4 weeks. The differentiated seedlings were transferred to a rooting medium for rooting culture; the rooted seedlings were subjected to acclimatization treatment, and then transferred to a pot containing soil for cultivation in a greenhouse; and overexpressed OsPPO1 L423S/Y425I and overexpressed OsPPO1 WT seedlings or events were obtained.

3. Detection of herbicide resistance of transgenic seedlings (T0 generation): different concentrations of compound A were sprayed to overexpressed rice OsPPO1 L423S/Y425I and OsPPO1 WT of T0 generation rice seedlings for resistance test. As shown in FIG. 24, compared with wild-type Huaidao No. 5, both the overexpressed rice OsPPO1 L423S/Y425I and the overexpressed OsPPO1 WT showed certain level of tolerance/resistance to compound A. Their resistance levels were similar at 45 g/ha application concentration, but at higher application concentrations such as 135 g/ha and 270 g/ha, the overexpressed OsPPO1 L423S/Y425I still showed resistance while the overexpressed OsPPO1 WT showed no difference with the wild-type control, indicating that rice with overexpressed OsPPO1 L423S/Y425I has a higher tolerance to compound A.

At the same time, through many tests, it is found that introducing the corresponding resistance site or combination in the present invention into other plants by transgenic technology or gene editing technique would confer tolerance to PPO-inhibiting herbicides as well, indicating that it has good industrial value.

All publications and patent applications mentioned in the description are incorporated herein by reference, as if each publication or patent application is individually and specifically incorporated herein by reference.

Although the aforementioned invention has been described in more details by way of examples and embodiments for clear understanding, it is obvious that certain changes and modifications can be implemented within the scope of the appended claims, and such changes and modifications are all within the scope of the present invention.

1. A PPO polypeptide or a bioactive fragment thereof tolerant to a PPO-inhibiting herbicide, which is characterized in that the polypeptide comprises the motif “LLLNYI”, wherein the leucine L at position 3 within the motif is substituted with any other amino acid, and/or the tyrosine Y at position 5 is substituted with any other amino acid.

2. The PPO polypeptide or a bioactive fragment thereof according to claim 1, which is characterized in that within the motif “LLLNYI”, the leucine L at position 3 is mutated to serine S; or
the leucine L at position 3 is mutated into isoleucine I; or
the leucine L at position 3 is mutated in glycine G; or
the leucine L at position 3 is mutated into threonine T; or
the leucine L at position 3 is mutated into valine V; or
the leucine L at position 3 is mutated into tryptophan W; or
the tyrosine Y at position 5 is mutated into methionine M; or
the tyrosine Y at position 5 is mutated into isoleucine I; or
the tyrosine Y at position 5 is mutated into leucine L; or
the tyrosine Y at position 5 is mutated into valine V.

3. (canceled)

4. The PPO polypeptide or a bioactive fragment thereof according to claim 1, which is characterized in that within the motif “LLLNYI”, the leucine L at position 3 is mutated to serine S and the tyrosine Y at position 5 is mutated into isoleucine I; or
the leucine L at position 3 is mutated into threonine T and the tyrosine Y at position 5 is mutated into isoleucine I; or
the leucine L at position 3 is mutated into threonine T and the tyrosine Y at position 5 is mutated into valine V; or
the leucine L at position 3 is mutated into serine S and the tyrosine Y at position 5 is mutated into valine V; or
the leucine L at position 3 is mutated into valine V and the tyrosine Y at position 5 is mutated into leucine L; or
the leucine L at position 3 is mutated into tryptophan W and the tyrosine Y at position 5 is mutated into leucine L.

5. The PPO polypeptide or a bioactive fragment thereof according to claim 1, wherein the polypeptide comprises a mutant of freely-combined amino acid sequence and a fragment thereof that has at least 60%, at least 65%, at least 70%, at least 75%, at least 80%, at least 85%, at least 90%, at least 95%, at least 96%, at least 97%, at least 98% or at least 99% sequence identity to an amino acid sequence as set forth in any one from SEQ ID NO: 1-19, and the mutant comprises one or more amino acid mutations selected from: (i) the leucine L at position 3 within the motif is substituted with any other amino acid, (ii) the tyrosine Y at position 5 is substituted with any other amino acid;
or, the polypeptide has an amino acid sequence as set forth in any one from SEQ ID NO: 1-19, except that it has one or more amino acid mutations selected from: (i) the leucine L at position 3 within the motif is substituted with any other amino acid, (ii) the tyrosine Y at position 5 is substituted with any other amino acid.

6. (canceled)

7. The PPO polypeptide or a bioactive fragment thereof according to claim 1, wherein, as compared to the amino acid sequence of a wild-type rice PPO1, the amino acid sequence of the PPO polypeptide has one or more mutations at one or more positions corresponding to 423 and 425 of the amino acid sequence of wild-type rice PPO1 protein as set forth in SEQ ID NO: 1; or
as compared to the amino acid sequence of a wild-type corn PPO1, the amino acid sequence of the PPO polypeptide has one or more mutations at one or more positions corresponding to 424 and 426 of the amino acid sequence of wild-type corn PPO1 protein as set forth in SEQ ID NO: 2; or
as compared to the amino acid sequence of a wild-type oilseed rape PPO1, the amino acid sequence of the PPO polypeptide has one or more mutations at one or more positions corresponding to 424 and 426 of the amino acid sequence of wild-type oilseed rape PPO1 protein as set forth in SEQ ID NO: 3; or
as compared to the amino acid sequence of a wild-type oilseed rape PPO1, the amino acid sequence of the PPO polypeptide has one or more mutations at one or more positions corresponding to 423 and 425 of the amino acid sequence of wild-type oilseed rape PPO1 protein as set forth in SEQ ID NO: 4; or
as compared to the amino acid sequence of a wild-type peanut PPO1, the amino acid sequence of the PPO polypeptide has one or more mutations at one or more positions corresponding to 445 and 447 of the amino acid sequence of wild-type peanut PPO1 protein as set forth in SEQ ID NO: 5; or
as compared to the amino acid sequence of a wild-type peanut PPO1, the amino acid sequence of the PPO polypeptide has one or more mutations at one or more positions corresponding to 439 and 441 of the amino acid sequence of wild-type peanut PPO1 protein as set forth in SEQ ID NO: 6; or
as compared to the amino acid sequence of a wild-type soybean PPO1, the amino acid sequence of the PPO polypeptide has one or more mutations at one or more positions corresponding to 430 and 432 of the amino acid sequence of wild-type soybean PPO1 protein as set forth in SEQ ID NO: 7; or
as compared to the amino acid sequence of a wild-type sorghum PPO1, the amino acid sequence of the PPO polypeptide has one or more mutations at one or more positions corresponding to 423 and 425 of the amino acid sequence of wild-type sorghum PPO1 protein as set forth in SEQ ID NO: 8; or
as compared to the amino acid sequence of a wild-type wheat PPO1, the amino acid sequence of the PPO polypeptide has one or more mutations at one or more positions corresponding to 418 and 420 of the amino acid sequence of wild-type wheat PPO1 protein as set forth in SEQ ID NO: 9, 10 or 11; or
as compared to the amino acid sequence of a wild-type tomato PPO1, the amino acid sequence of the PPO polypeptide has one or more mutations at one or more positions corresponding to 445 and 447 of the amino acid sequence of wild-type tomato PPO1 protein as set forth in SEQ ID NO: 12; or
as compared to the amino acid sequence of a wild-type potato PPO1, the amino acid sequence of the PPO polypeptide has one or more mutations at one or more positions corresponding to 444 and 446 of the amino acid sequence of wild-type potato PPO1 protein as set forth in SEQ ID NO: 13; or
as compared to the amino acid sequence of a wild-type tobacco PPO1, the amino acid sequence of the PPO polypeptide has one or more mutations at one or more positions corresponding to 440 and 442 of the amino acid sequence of wild-type tobacco PPO1 protein as set forth in SEQ ID NO: 14; or
as compared to the amino acid sequence of a wild-type Arabidopsis thaliana PPO1, the amino acid sequence of the PPO polypeptide has one or more mutations at one or more positions corresponding to 423 and 425 of the amino acid sequence of wild-type Arabidopsis thaliana PPO1 protein as set forth in SEQ ID NO: 15; or
as compared to the amino acid sequence of a wild-type upland cotton PPO1, the amino acid sequence of the PPO polypeptide has one or more mutations at one or more positions corresponding to 426 and 428 of the amino acid sequence of wild-type upland cotton PPO1 protein as set forth in SEQ ID NO: 16; or
as compared to the amino acid sequence of a wild-type radish PPO1, the amino acid sequence of the PPO polypeptide has one or more mutations at one or more positions corresponding to 425 and 427 of the amino acid sequence of wild-type radish PPO1 protein as set forth in SEQ ID NO: 17; or
as compared to the amino acid sequence of a wild-type foxtail millet PPO1, the amino acid sequence of the PPO polypeptide has one or more mutations at one or more positions corresponding to 422 and 424 of the amino acid sequence of wild-type foxtail millet PPO1 protein as set forth in SEQ ID NO: 18; or
as compared to the amino acid sequence of a wild-type cabbage PPO1, the amino acid sequence of the PPO polypeptide has one or more mutations at one or more positions corresponding to 424 and 426 of the amino acid sequence of wild-type cabbage PPO1 protein as set forth in SEQ ID NO: 19.

8. The PPO polypeptide or a bioactive fragment thereof according to claim 7, wherein, as compared to the amino acid sequence of a wild-type rice PPO1, the amino acid sequence of the PPO polypeptide has one or more mutations selected from the group consisting of L423S, L423I, L423G, Y425M, Y425I and Y425V at one or more positions corresponding to 423 and 425 of the amino acid sequence of wild-type rice PPO1 protein as set forth in SEQ ID NO: 1; preferably, it has the following mutations: L423S/Y425I; or
as compared to the amino acid sequence of a wild-type corn PPO1, the amino acid sequence of the PPO polypeptide has one or more mutations selected from the group consisting of L424T, L424S, L424V, Y424W, Y426V, Y426I and Y426L at one or more positions corresponding to 424 and 426 of the amino acid sequence of wild-type corn PPO1 protein as set forth in SEQ ID NO: 2; preferably, it has the following mutations: L424T/Y426V, L424S/Y426V, L424V/Y426L, L424W/Y426L or L424S/Y426I; or
as compared to the amino acid sequence of a wild-type oilseed rape PPO1, the amino acid sequence of the PPO polypeptide has one or more mutations selected from the group consisting of L424S and Y426I at one or more positions corresponding to 424 and 426 of the amino acid sequence of wild-type oilseed rape PPO1 protein as set forth in SEQ ID NO: 3; preferably, it has the following mutations: L424S/Y426I; or
as compared to the amino acid sequence of a wild-type oilseed rape PPO1, the amino acid sequence of the PPO polypeptide has one or more mutations selected from the group consisting of L423S and Y425I at one or more positions corresponding to 423 and 425 of the amino acid sequence of wild-type oilseed rape PPO1 protein as set forth in SEQ ID NO: 4; preferably, it has the following mutations: L423S/Y425I; or
as compared to the amino acid sequence of a wild-type peanut PPO1, the amino acid sequence of the PPO polypeptide has one or more mutations selected from the group consisting of L445S and Y447I at one or more positions corresponding to 445 and 447 of the amino acid sequence of wild-type peanut PPO1 protein as set forth in SEQ ID NO: 5; preferably, it has the following mutations: L445S/Y447I; or
as compared to the amino acid sequence of a wild-type peanut PPO1, the amino acid sequence of the PPO polypeptide has one or more mutations selected from the group consisting of L439S and Y441I at one or more positions corresponding to 439 and 441 of the amino acid sequence of wild-type peanut PPO1 protein as set forth in SEQ ID NO: 6; preferably, it has the following mutations: L439S/Y441I; or
as compared to the amino acid sequence of a wild-type soybean PPO1, the amino acid sequence of the PPO polypeptide has one or more mutations selected from the group consisting of L430S and Y432I at one or more positions corresponding to 430 and 432 of the amino acid sequence of wild-type soybean PPO1 protein as set forth in SEQ ID NO: 7; preferably, it has the following mutations: L430S/Y432I; or
as compared to the amino acid sequence of a wild-type sorghum PPO1, the amino acid sequence of the PPO polypeptide has one or more mutations selected from the group consisting of L423S and Y425I at one or more positions corresponding to 423 and 425 of the amino acid sequence of wild-type sorghum PPO1 protein as set forth in SEQ ID NO: 8; preferably, it has the following mutations: L423S/Y425I; or
as compared to the amino acid sequence of a wild-type wheat PPO1, the amino acid sequence of the PPO polypeptide has one or more mutations selected from the group consisting of L418S and Y420I at one or more positions corresponding to 418 and 420 of the amino acid sequence of wild-type wheat PPO1 protein as set forth in SEQ ID NO: 9, 10 or 11; preferably, it has the following mutations: L418S/Y420I; or
as compared to the amino acid sequence of a wild-type tomato PPO1, the amino acid sequence of the PPO polypeptide has one or more mutations selected from the group consisting of L445S and Y447I at one or more positions corresponding to 445 and 447 of the amino acid sequence of wild-type tomato PPO1 protein as set forth in SEQ ID NO: 12; preferably, it has the following mutations: L445S/Y447I; or
as compared to the amino acid sequence of a wild-type potato PPO1, the amino acid sequence of the PPO polypeptide has one or more mutations selected from the group consisting of L444S and Y446I at one or more positions corresponding to 444 and 446 of the amino acid sequence of wild-type potato PPO1 protein as set forth in SEQ ID NO: 13; preferably, it has the following mutations: L444S/Y446I; or
as compared to the amino acid sequence of a wild-type tobacco PPO1, the amino acid sequence of the PPO polypeptide has one or more mutations selected from the group consisting of L440S and Y442I at one or more positions corresponding to 440 and 442 of the amino acid sequence of wild-type tobacco PPO1 protein as set forth in SEQ ID NO: 14; preferably, it has the following mutations: L440S/Y442I; or
as compared to the amino acid sequence of a wild-type Arabidopsis thaliana PPO1, the amino acid sequence of the PPO polypeptide has one or more mutations selected from the group consisting of L423S and Y425I at one or more positions corresponding to 423 and 425 of the amino acid sequence of wild-type Arabidopsis thaliana PPO1 protein as set forth in SEQ ID NO: 15; preferably, it has the following mutations: L423S/Y425I; or
as compared to the amino acid sequence of a wild-type upland cotton PPO1, the amino acid sequence of the PPO polypeptide has one or more mutations selected from the group consisting of L426S and Y428I at one or more positions corresponding to 426 and 428 of the amino acid sequence of wild-type upland cotton PPO1 protein as set forth in SEQ ID NO: 16; preferably, it has the following mutations: L426S/Y428I; or
as compared to the amino acid sequence of a wild-type radish PPO1, the amino acid sequence of the PPO polypeptide has one or more mutations selected from the group consisting of L425S and Y427I at one or more positions corresponding to 425 and 427 of the amino acid sequence of wild-type radish PPO1 protein as set forth in SEQ ID NO: 17; preferably, it has the following mutations: L425S/Y427I; or
as compared to the amino acid sequence of a wild-type foxtail millet PPO1, the amino acid sequence of the PPO polypeptide has one or more mutations selected from the group consisting of L422S and Y424I at one or more positions corresponding to 422 and 424 of the amino acid sequence of wild-type foxtail millet PPO1 protein as set forth in SEQ ID NO: 18; preferably, it has the following mutations: L422S/Y424I; or
as compared to the amino acid sequence of a wild-type cabbage PPO1, the amino acid sequence of the PPO polypeptide has one or more mutations selected from the group consisting of L424S and Y426I at one or more positions corresponding to 424 and 426 of the amino acid sequence of wild-type cabbage PPO1 protein as set forth in SEQ ID NO: 19;
preferably, it has the following mutations: L424S/Y426I.

9. The PPO polypeptide or a bioactive fragment thereof according to claim 8, wherein the polypeptide has an amino acid sequence as set forth in any one from SEQ ID NO: 20-48.

10. An isolated polynucleotide comprising a nucleic acid sequence selected from:
(1) a nucleic acid sequence encoding the PPO polynucleotide or a bioactive fragment thereof according to claim 1, or a partial sequence thereof or a complementary sequence thereof;
(2) a nucleic acid sequence that hybridizes to the sequence shown in (1) under stringent conditions; and
(3) a nucleic acid sequence encoding the same amino acid sequence as the sequence shown in (1) due to degeneracy of genetic code, or a complementary sequence thereof;
preferably, the polynucleotide being a DNA molecule.

11. A plant genome comprising the polynucleotide according to claim 10.

12. A vector construct comprising the polynucleotide according to claim 10 and the homologous or non-homologous promoter operably linked thereto.

13. A host cell comprising the polynucleotide according to claim 10 or a vector construct comprising the polynucleotide; preferably, the host cell being a plant cell.

14. A producing method of a plant cell to gain or improve its tolerance to a PPO-inhibiting herbicide, comprising producing the polynucleotide according to claim 10 or a vector construct comprising the polynucleotide in the plant cell by using gene editing method, or introducing the polynucleotide or the vector construct into the plant cell by using transgenic methods.

15. A producing method of a plant to gain or improve its tolerance to a PPO-inhibiting herbicide, comprising regenerating a plant cell into a plant, wherein the plant cell comprises the polynucleotide according to claim 10 or a vector construct comprising the polynucleotide, or the plant cell is produced by the following method: producing the polynucleotide or the vector construct in the plant cell by using gene editing methods, or introducing the polynucleotide or the vector construct into the plant cell by using transgenic methods.

16. A plant produced by the method according to claim 15.

17. A method of enabling a plant to gain or improve tolerance to a PPO-inhibiting herbicide, comprising introducing a modification into the gene encoding a protein with PPO activity to produce the PPO polypeptide or a bioactive fragment thereof according to claim 1.

18. A method of gaining or improving the tolerance of a plant cell, plant tissue, plant part or plant to a PPO-inhibiting herbicide, comprising expressing the PPO polypeptide or a bioactive fragment thereof according to claim 1 in the plant cell, plant tissue, plant part or plant;
or, comprising hybridizing a plant expressing the PPO polypeptide or a bioactive fragment thereof with another plant, and screening of a plant or a part thereof capable of gaining or improving the tolerance to a PPO-inhibiting herbicide;
or, comprising gene editing a protein with PPO activity of the plant cell, plant tissue, plant part or plant to achieve expression of the PPO polypeptide or a bioactive fragment thereof.

19. (canceled)

20. A method for controlling weeds in a plant cultivation site, comprising applying to the cultivation site a herbicidally effective amount of PPO-inhibiting herbicide, wherein:
(i) the plant is regenerated from a plant cell, wherein the plant cell comprises an isolated polynucleotide or a vector construct comprising the polynucleotide which comprises a nucleic acid sequence selected from:
(ia) a nucleic acid sequence encoding the PPO polynucleotide or a bioactive fragment thereof according to claim 1, or a partial sequence thereof or a complementary sequence thereof;
(ib) a nucleic acid sequence that hybridizes to the sequence shown in (ia) under stringent conditions; and
(ic) a nucleic acid sequence encoding the same amino acid sequence as the sequence shown in (1) due to degeneracy of genetic codes, or a complementary sequence thereof;
or,
(ii) the plant is regenerated from a plant cell, wherein the plant cell is produced by the following method: producing the polynucleotide or the vector construct as defined in (i) in the plant cell by using gene editing methods, or introducing the polynucleotide or the vector construct into the plant cell by using transgenic methods;
or,
(iii) the plant is produced by introducing a modification into the gene encoding a protein with PPO activity to produce the PPO polypeptide or a bioactive fragment thereof in the plant;
or,
(iv) the plant is produced by expressing the PPO polypeptide or a bioactive fragment thereof in the plant;
or,
(v) the plant is produced by hybridizing a plant expressing the PPO polypeptide or a bioactive fragment thereof with another plant, and screening a plant or a part thereof capable of gaining or improving the tolerance to a PPO inhibitor herbicide;
or,
(vi) the plant is produced by gene editing a protein with PPO activity of the plant to achieve expression of the PPO polypeptide or a bioactive fragment thereof.

21. The method according to claim 20, wherein the PPO-inhibiting herbicide is applied in combination with one or more additional herbicides.

22. The plant genome according to claim 11, wherein the plant is a monocotyledonous or dicotyledonous plant; preferably, the plant is rice (Oryza sativa L.), sorghum (Sorghum bicolor), wheat (Triticum aestivum), barley (Hordeum vulgare), foxtail millet (Setaria italica), corn (Zea mays), sugarcane (Saccharum officinarum), Arabidopsis thaliana, soybean (Glycine max), peanut (Arachis hypogaea), tobacco (Nicotiana tabacum), cotton (Gossypium hirsutum), radish (Raphanus sativus), cabbage (Brassica oleracea), sweet potato (Dioscorea esculenta), yam (Dioscorea cayenensis), cassava (Manihot esculenta), potato (Solanum tuberosum), tomato (Solanum lycopersicum), pepper (Capsicum annuum), eggplant (Solanum melongena), watermelon (Citrullus lanatus), squash (Cucurbita moschata), cucumber (Cucumis sativus), lettuce (Lactuca sativa), sesame (Sesamum indicum), oilseed rape (Brassica napus), sunflower (Helianthus annuus), mulberry (Morus alba), cowpea (Vigna unguiculata), strawberry (Fragaria ananassa), apple (Malus domestica), peach (Prunus persica), cherry (Prunus pseudocerasus), apricot (Prunus armeniaca), grape vine (Vitis vinifera), papaya (Carica papaya) or alfalfa (Medicago sativa).

23. The method according to claim 15, wherein the PPO-inhibiting herbicide is one or more compounds selected from a group consisting of pyrimidinediones, diphenyl-ethers, phenylpyrazoles, N-phenylphthalimides, thiadiazoles, oxadiazoles, triazolinones, oxazolidinediones, and others; preferably,
(1) the pyrimidinediones include: butafenacil, saflufenacil benzfendizone, tiafenacil, [3-[2-Chloro-4-fluoro-5-(1-methyl-6-trifluoromethyl-2,4-dioxo-1,2,3,4-tetrahydropyrimidin-3-yl) phenoxy]-2-pyridyloxy]acetic acid ethyl ester, 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, 3-[7-chloro-5-fluoro-2-(trifluoromethyl)-1H-benzimidazol-4-yl]-1-methyl-6-(trifluoromethyl)-1H-pyrimidine-2,4-dione, flupropacil, and 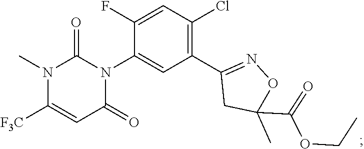 [SMILES: CCOC(=O)C1(C)CC(c2cc(-n3c(=O)cc(C(F)(F)F)n(C)c3=O)c(F)cc2Cl)=NO1]
(2) the diphenyl-ethers include: fomesafen, oxyfluorfen, aclonifen, lactofen, chlomethoxyfen, chlomitrofen, fluoroglycofen-ethyl, acifluorfen or sodium salt, bifenox, ethoxyfen, ethoxyfen-ethyl, fluoronitrofen, furyloxyfen, nitrofluorfen, and halosafen;
(3) the phenylpyrazoles include: pyraflufen-ethyl, and fluazolate;
(4) the N-phenylphthalimides include: flumioxazin, cinidon-ethyl, flumipropyn, and flumiclorac-pentyl;
(5) the thiadiazoles include: fluthiacet-methyl, fluthiacet, and thidiazimin;
(6) the oxadiazoles include: oxadiargyl, and oxadiazon;
(7) the triazolinones include: carfentrazone, carfentrazone-ethyl, sulfentrazone, azafenidin, and bencarbazone;
(8) the oxazolidinediones include: pentoxazone;
(9) the others include: pyraclonil, flufenpyr-ethyl, profluazol, trifludimoxazin, N-ethyl-3-(2,6-dichloro-4-trifluoromethylphenoxy)-5-methyl-TH-pyrazole-1-carboxamide, N-tetrahydrofurfuryl-3-(2,6-dichloro-4-trifluoromethylphenoxy)-5-methyl-1H-pyrazole-1-carboxamide, N-ethyl-3-(2-chloro-6-fluoro-4-trifluoromethylphenoxy)-5-methyl-1H-pyrazole-1-carboxamide, N-tetrahydrofurfuryl-3-(2-chloro-6-fluoro-4-trifluoromethylphenoxy)-5-methyl-1H-pyrazole-1-carboxamide, 3-[7-Fluoro-3-oxo-4-(prop-2-ynyl)-3,4-dihydro-2H-benzo[1,4]oxazin-6-yl]-1,5-dimethyl-6-thioxo[1,3,5]triazinane-2,4-dione, 2-(2,2,7-Trifluoro-3-oxo-4-prop-2-ynyl-3,4-dihydro-2H-benzo[1,4]oxazin-6-yl)-4,5,6,7-tetrahydroisoindole-1,3-dione, methyl (E)-4-[2-chloro-5-[4-chloro-5-(difluoromethoxy)-1H-methylpyrazol-3-yl]-4-fluorophenoxy]-3-methoxy-but-2-enoate, phenylpyridines, benzoxazinone derivatives and compounds represented by general formula I 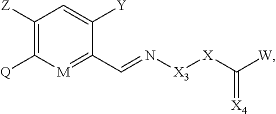
wherein,
Q represents 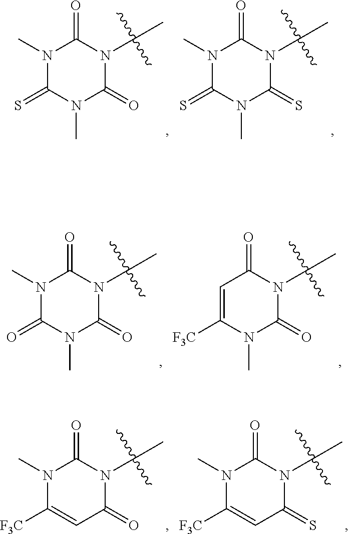 [SMILES: *n1c(=O)n(C)c(=S)n(C)c1=O ; *n1c(=O)n(C)c(=S)n(C)c1=S ; *n1c(=O)n(C)c(=O)n(C)c1=O ; *n1c(=O)cc(C(F)(F)F)n(C)c1=O ; *n1c(=O)cc(C(F)(F)F)n(C)c1=O ; *n1c(=S)cc(C(F)(F)F)n(C)c1=O] 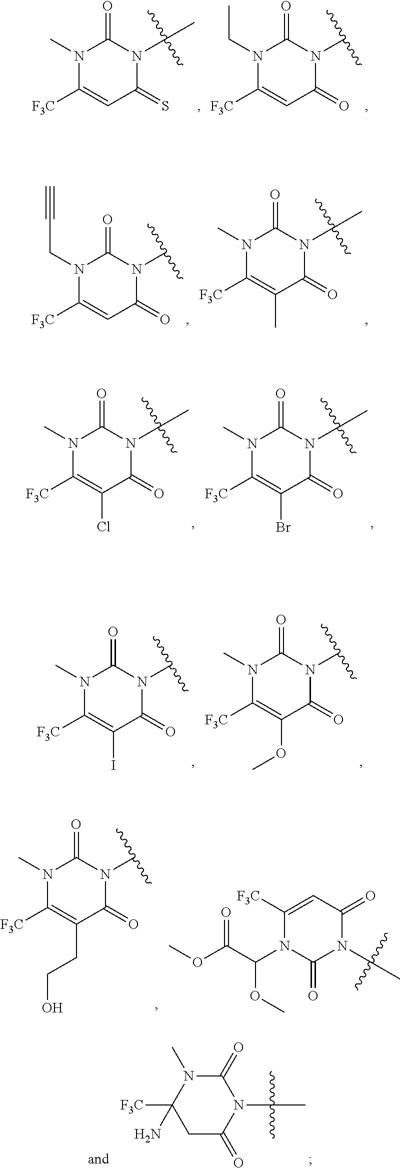
Y represents halogen, halogenated C1-C6 alkyl or cyano;
Z represents halogen;
M represents CH or N;
X represents —CX₁X₂—(C1-C6 alkyl)ₙ-, —(C1-C6 alkyl)-CX₁X₂—(C1-C6 alkyl)ₙ- or —(CH₂)r-, n represents 0 or 1, r represents an integer greater than or equal to 2;
X₁ and X₂ independently represent hydrogen, halogen, C1-C6 alkyl, C2-C6 alkenyl, C2-C6 alkynyl, halogenated C1-C6 alkyl, halogenated C2-C6 alkenyl, halogenated C2-C6 alkynyl, C3-C6 cycloalkyl, C3-C6 cycloalkyl C1-C6 alkyl, C1-C6 alkoxy, C1-C6 alkylsulphanyl, hydroxy C1-C6 alkyl, C1-C6 alkoxy C1-C6 alkyl, phenyl or benzyl;
X₃ and X₄ independently represent O or S;
W represents hydroxyl, C1-C6 alkoxy, C2-C6 alkenyloxy, C2-C6 alkynyloxy, halogenated C1-C6 alkoxy, halogenated C2-C6 alkenyloxy, halogenated C2-C6 alkynyloxy, C3-C6 cycloalkyloxy, phenoxy, sulfydryl, C1-C6 alkylsulphanyl, C2-C6 alkenylsulphanyl, C2-C6 alkynylsulphanyl, halogenated C1-C6 alkylsulphanyl, halogenated C2-C6 alkenylsulphanyl, halogenated C2-C6 alkynylsulphanyl, C3-C6 cycloalkylsulphanyl, phenylsulphanyl, amino or C1-C6 alkylamino;
more preferably, Q represent 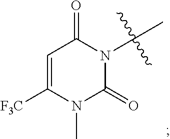 [SMILES: *n1c(=O)cc(C(F)(F)F)n(C)c1=O]
Y represents chlorine; Z represents fluorine; M represents CH; X represents —C*X₁X₂—(C1-C6 alkyl)ₙ-, n represents 0; X₁ represents hydrogen; X₂ represents methyl; X₃ and X₄ independently represent 0; W represents methoxy; wherein, C* is chiral center, and the compound is R configuration.
